(19) United States
(12) Patent Application Publication
(10) Pub. No.: US 2026/0130966 A1
(43) Pub. Date: May 14, 2026

(54) NOVEL THERAPEUTICS FOR PARASITIC INFECTION

(71) Applicants: UNIVERSITY OF SOUTH FLORIDA, Tampa, FL (US); UNIVERSITY OF FLORIDA RESEARCH FOUNDATION, INCORPORATED, Gainesville, FL (US)

(72) Inventors: Bill J. BAKER, Temple Terrace, FL (US); Joe BRACEGIRDLE, Tampa, FL (US); James R. ROCCA, Gainesville, FL (US); John H ADAMS, Tampa, FL (US); Nerida G. WILSON, Cottesloe (AU); Debora CASANDRA, Tampa, FL (US); Xingmin SUN, Tampa, FL (US); Anne-Claire D. LIMON, Tampa, FL (US)

(21) Appl. No.: 19/121,056

(22) PCT Filed: Oct. 16, 2023
(86) PCT No.: PCT/US2023/077024

§ 371 Date: Apr. 14, 2025

Related U.S. Application Data
(60) Provisional application No. 63/416,427, filed on Oct. 14, 2022.
(60) Provisional application No. 63/493,278, filed on Mar. 30, 2023.

(51) Int. Cl. A61K 38/08; A61K 31/015; A61K 31/365; A61K 31/575; A61P 33/06

(52) U.S. Cl. CPC A61K 38/08; A61K 31/015; A61K 31/365; A61K 31/575; A61P 33/06

(57) ABSTRACT

Described herein are compounds, formulations, and methods for blocking sporozoite invasion and subsequent liver-stage parasite development of a protozoan parasite, such as Plasmodium falciparum. Also described herein are compounds, formulations, and methods for inhibiting, treating, and preventing infection from a pathogenic bacterium, such as Clostridium difficile or a protist, such as Leishmania donovani.

### CROSS-REFERENCE TO RELATED APPLICATIONS

This application claims the benefit of and priority of U.S. Provisional Patent Application No. 63/493,278, filed Mar. 30, 2023, and U.S. Provisional Patent Application No. 63/416,427, filed Oct. 14, 2022. The content of each of the above-referenced applications is incorporated by reference herein in its entirety.

### STATEMENT REGARDING FEDERALLY SPONSORED RESEARCH OR DEVELOPMENT

This invention was made with government support under ANT1043749, ANT-0838776 and PLR1341339 awarded by the National Science Foundation, and All 17017 awarded by the National Institute of Health. The Government has certain rights in the invention.

### BACKGROUND

Plasmodium falciparum is a unicellular protozoan parasite of humans, and the deadliest species of Plasmodium that cause malaria in humans. It is transmitted through the bite of a female Anopheles mosquito. It is responsible for roughly 50% of all malaria cases. It causes the disease's most dangerous form called falciparum malaria. It is therefore regarded as the deadliest parasite in humans, causing 627,000 deaths in 2020. It is also associated with the development of blood cancer (Burkitt's lymphoma) and is classified as Group 2A carcinogen. Therefore, there is an urgent need for new treatments against Plasmodium infections.

More than 200,000 cases of Clostridium difficile infection occur in the US each year, causing abdominal distress, fever and diarrhea. Severe damage to the intestinal tract may result and some cases are fatal. Complicating treatment efforts, multidrug-resistant strains of C. difficile, often termed “superbugs”, have recently appeared, for which there are few, if any, treatments. Therefore, there is an urgent need for new treatments against C. difficile infections.

### SUMMARY

Described herein are compounds, formulations, and methods for inhibiting and/or preventing sporozoite invasion and subsequent liver-stage parasite development of a protozoan parasite, such as Plasmodium falciparum, including drug-resistant Plasmodium falciparum. Also described herein are compounds, formulations, and methods for inhibiting, treating and/or preventing C. difficile infections, including drug- and multidrug-resistant C. difficile infections.

The details of one or more embodiments of the invention are set forth in the accompanying drawings and the description below. Other features, objects, and advantages of the invention will be apparent from the description and drawings, and from the claims.

### DESCRIPTION OF THE FIGURES

FIG. 1 shows HRESIMS analysis of Compound (P2).

FIG. 2 shows an ¹H NMR spectrum analysis of Compound (P2).

FIG. 3 shows an HMBC NMR spectrum of Compound (P2).

FIG. 4A shows HRESIMSMS analysis of Compound (P2).

FIG. 4B shows proposed structures of fragment ions of Compound (P2).

FIG. 5 shows HRESIMS fragmentation of Compound (P2).

FIG. 6 is the chemical structure of Compound (P2). Important COSY (bold) and HMBC (→) correlations are shown.

FIG. 7A shows HRESIMSMS analysis of Compound (P1).

FIG. 7B shows proposed structures of Compound (P1) fragment ions.

FIG. 8 shows HRESIMS analysis of Compound (P3).

FIG. 9A shows HRESIMSMS analysis of Compound (P3).

FIG. 9B shows proposed structures of fragment ions of Compound (P3).

FIG. 10 shows HRESIMS analysis of Compound (P4).

FIG. 11 shows ¹H NMR spectrum analysis of Compound (P4).

FIG. 12 shows HRESIMS analysis of Compound (P5).

FIG. 13 shows HRESIMS analysis of Compound (P6).

FIG. 14 shows a ¹³C APT (Attached Proton Test) NMR spectrum (200 MHz, CD₃OD) of Compound (P6).

FIG. 15 shows an ¹H NMR spectrum (500 MHz, DMSO-d₆) of Compound (P6).

FIG. 16 shows an HRESIMS analysis of Compound (P7).

FIG. 17A shows HRESIMSMS analysis of Compound (P7).

FIG. 17B shows proposed fragment ions of Compound (P7).

FIG. 18 shows an ¹H NMR spectrum (500 MHz, DMSO-d₆) of Compound (P7).

FIG. 19 shows an HRESIMS analysis of Compound (P8).

FIG. 20 shows an ¹H NMR spectrum (500 MHz, DMSO-d₆) of Compound (P8).

FIG. 21 shows ¹³C APT (Attached Proton Test) NMR spectrum (200 MHz, CD₃OD) of Compound (P8) in both compact (A) and extended (B-D) representations.

FIG. 22 is an illustrated set of chemical structures representing terpenoids isolated from a deep-water Antarctic octocoral Alcyonium sp. The set of chemical structures is divided into three groups: formula I, which includes alcyopterosins T, U, C, G, and O; formula II, which includes alcyopterosins V, E, and L; and a group containing the single entity, alcyosterone.

FIG. 23 is a set of drawings illustrating HMBC correlations establishing the planar structure of alcyopterosin T (C1), alcyopterosin U (C2), and alcyopterosin V (C3).

FIG. 24 is a drawing illustrating Key HMBC (→) and COSY (−) correlations for alcyosterone (C5).

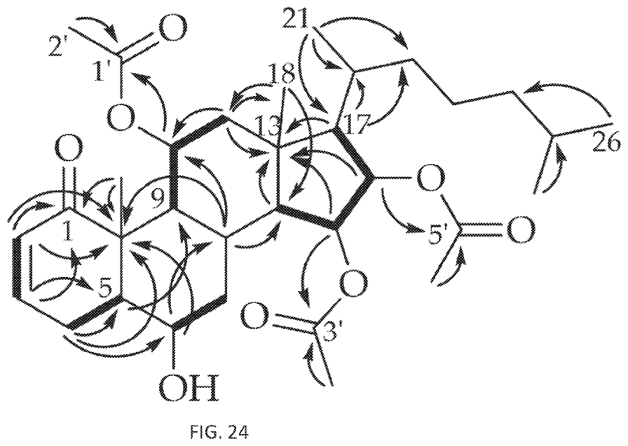

FIG. 25 is a drawing illustrating an MM2 energy-minimized structure overlaid with ROESY relationships which established many of the relative configurational relationships of alcyosterone (C5).

FIG. 26 is a drawing illustrating an asymmetric unit of alcyosterone (CS) with anisotropic displacement parameters drawn at 50% probability level.

FIG. 27 is a graph illustrating maximum Likelihood tree topology comparing msh1 sequences of Alcyonium specimen with those available on Genbank.

FIG. 28 is a graph illustrating a ¹H NMR spectrum of alcyopterosin T (C1), 500 MHz, CDC₁₃.

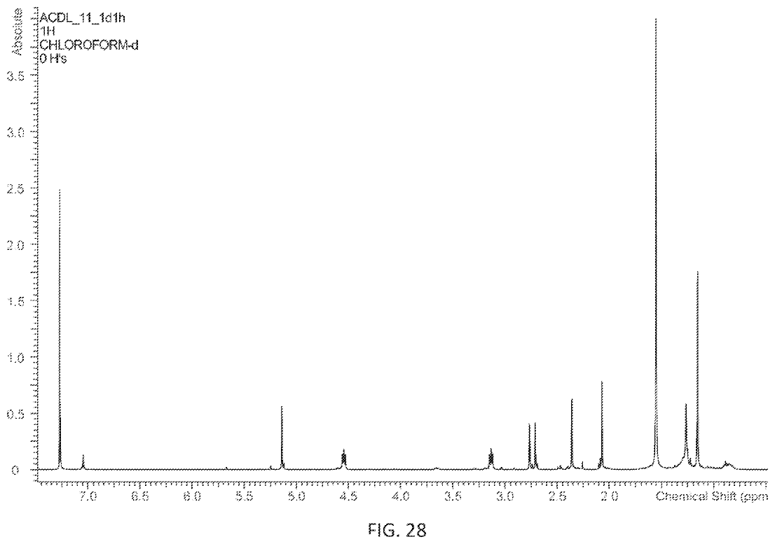

FIG. 29 is a graph illustrating a COSY spectrum of alcyopterosin T (C1), 500 MHz, CDC₁₃.

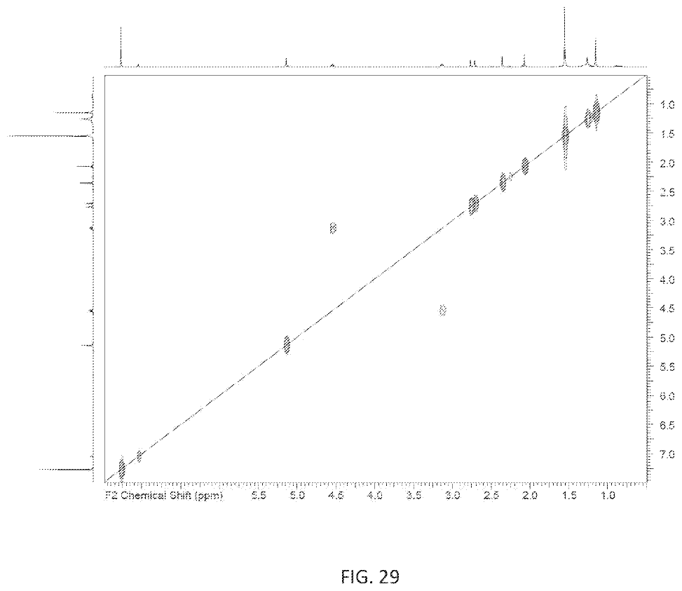

FIG. 30 is a graph illustrating an HSQC spectrum of alcyopterosin T (C1), 500 MHz, CDC₁₃.

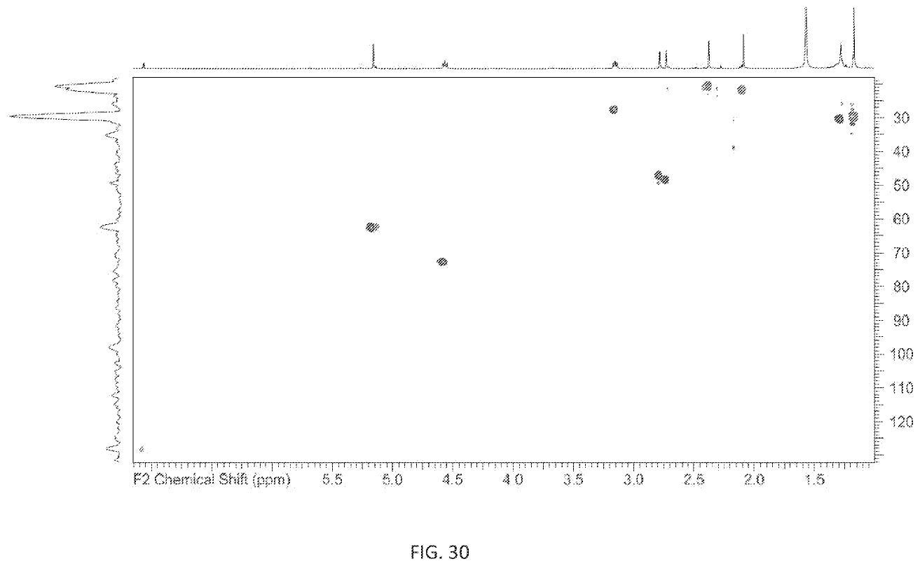

FIG. 31 is a graph illustrating an HMBC spectrum of alcyopterosin T (C1), 500 MHz, CDC₁₃.

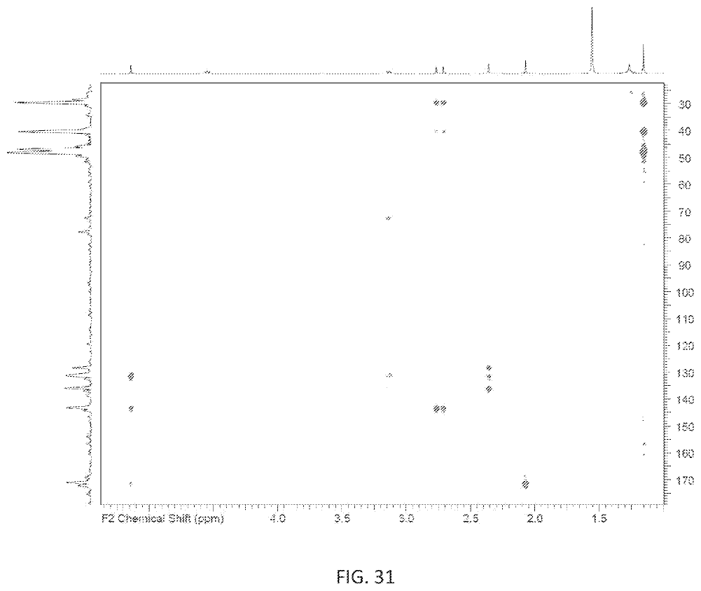

FIG. 32 is a graph illustrating an HRESIMS of alcyopterosin T (C1). Calculated for C₁₇H₂₃NO₅Na, 344.1468.

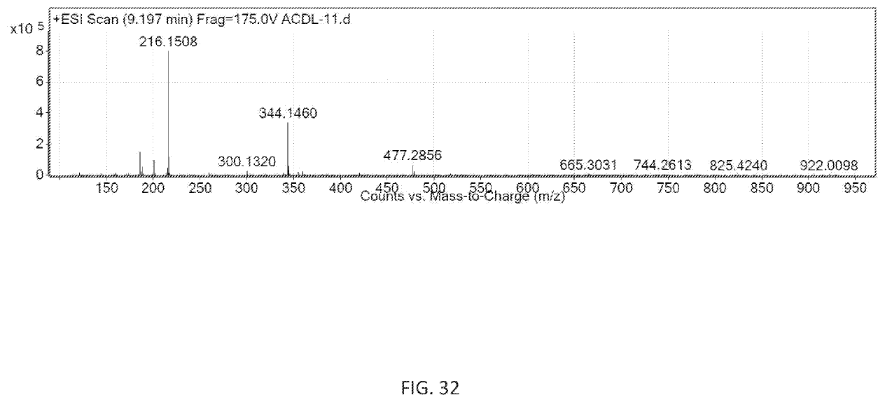

FIG. 33 is a graph illustrating a ¹H NMR spectrum of alcyopterosin U (C2), 500 MHz, CDC₁₃.

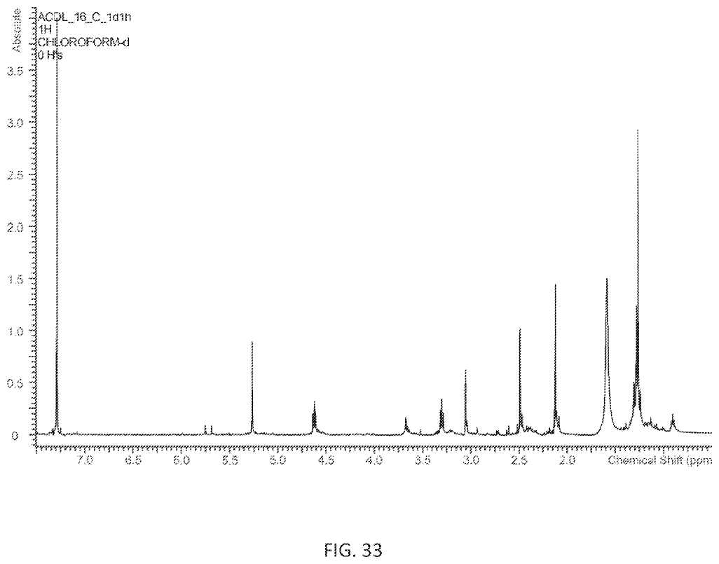

FIG. 34 is a graph illustrating a COSY spectrum of alcyopterosin U (C2), 500 MHz, CDC₁₃.

FIG. 35 is a graph illustrating HSQC spectrum of alcyopterosin U (C2), 500 MHz, CDC₁₃.

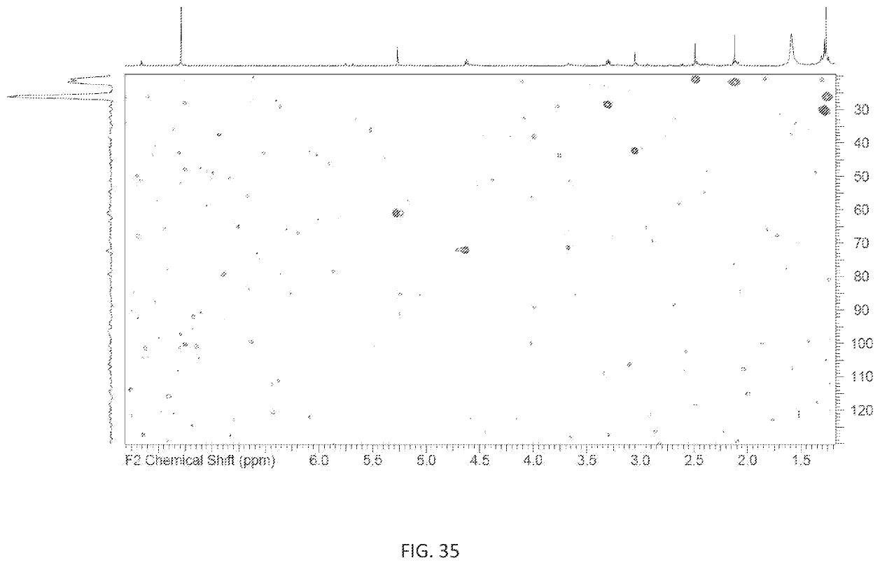

FIG. 36 is a graph illustrating HMBC spectrum of alcyopterosin U (C2), 500 MHz, CDC₁₃.

FIG. 37 is a graph illustrating an HRESIMS of alcyopterosin U (C2). Calculated for C₁₇H₂₁NO₆H, 336.1442.

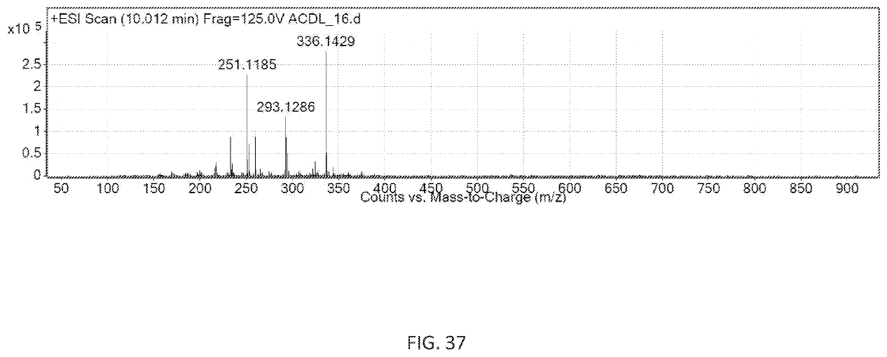

FIG. 38 is a graph illustrating a ¹H NMR spectrum of alcyopterosin V (C3), 500 MHz, CDC₁₃.

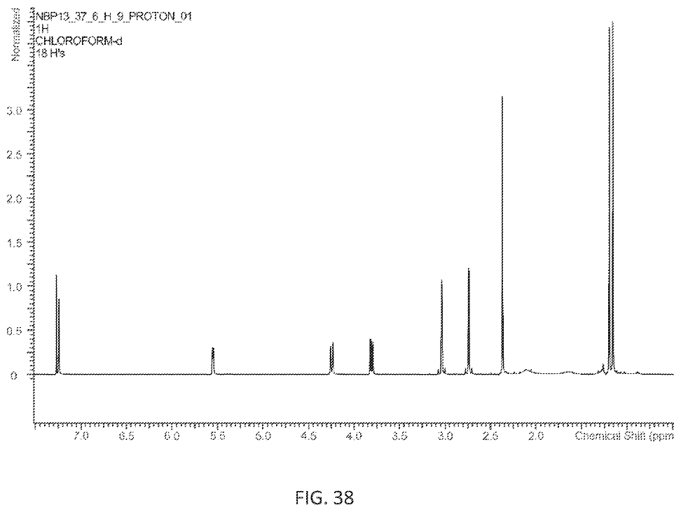

FIG. 39 is a graph illustrating a ¹³C NMR spectrum of alcyopterosin V (C3), 125 MHz, CDC₁₃.

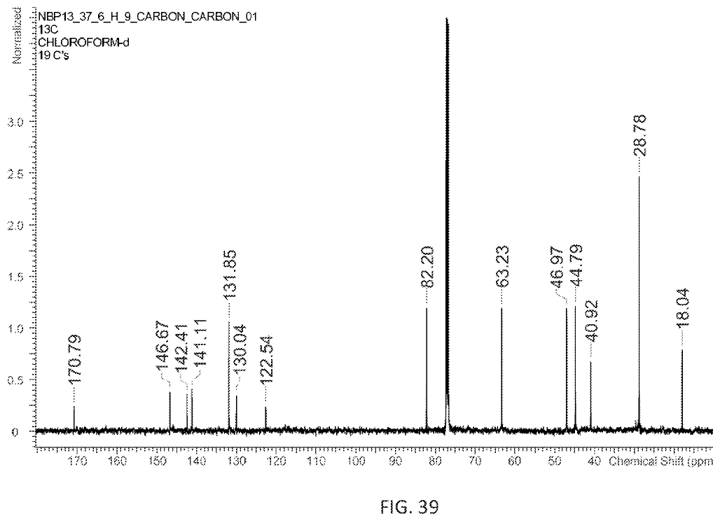

FIG. 40 is a graph illustrating a COSY spectrum of alcyopterosin V (C3), 500 MHz, CDC₁₃.

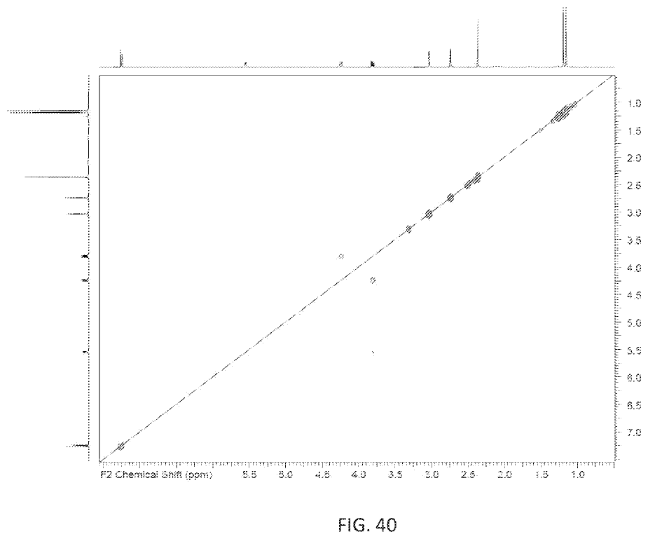

FIG. 41 is a graph illustrating an HSQC spectrum of alcyopterosin V (C3), 500 MHz, CDC₁₃.

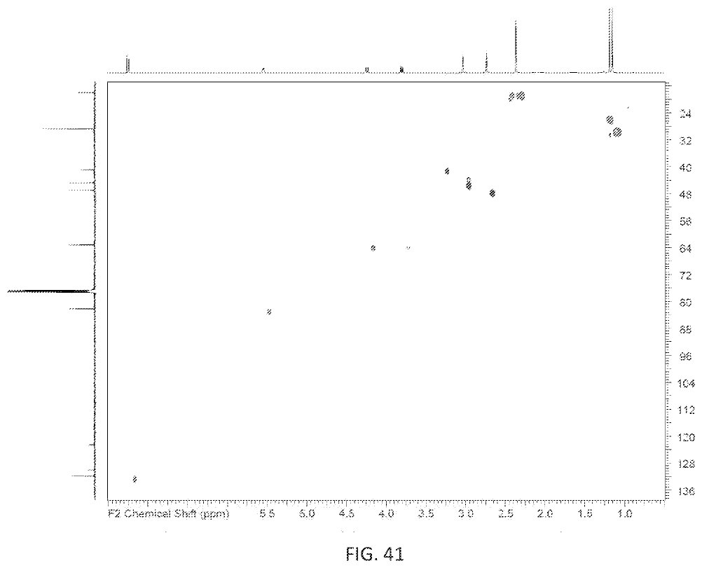

FIG. 42 is a graph illustrating HMBC spectrum of alcyopterosin V (C3), 500 MHz, CDC₁₃.

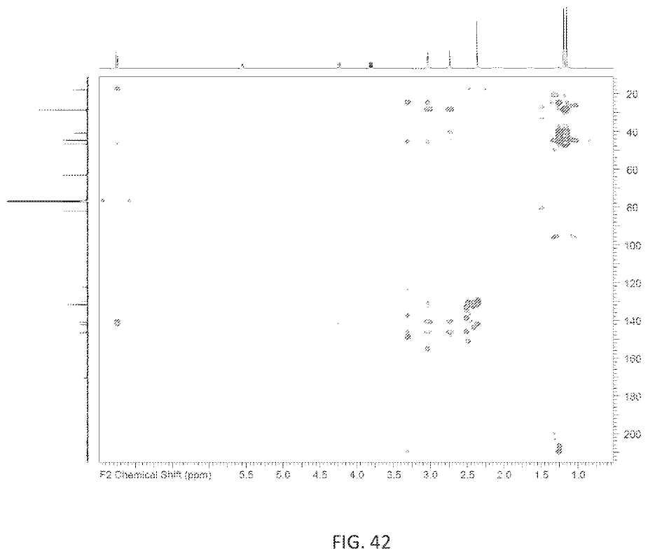

FIG. 43 is a graph illustrating a blank subtracted HRESIMS of alcyopterosin V (C3). Calculated for C₁₅H₁₅O₃H, 247.1329.

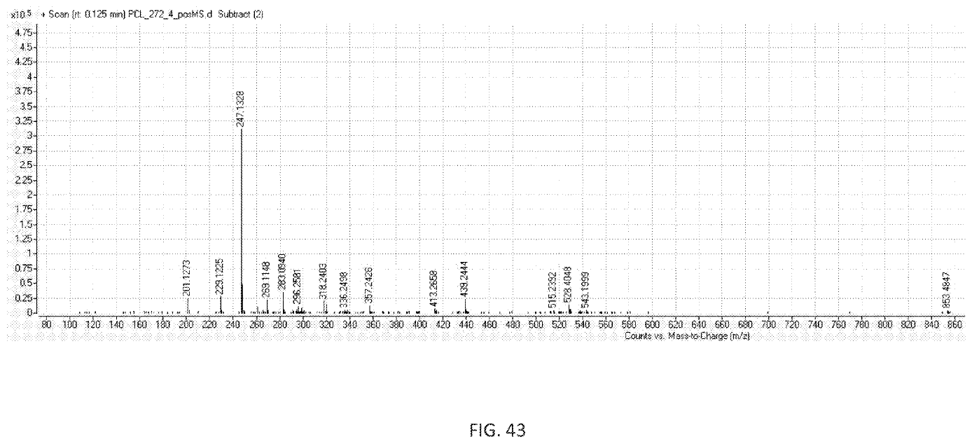

FIG. 44 is a graph illustrating a ¹H NMR spectrum of alcyosterone (C5), 600 MHz, CDC₁₃.

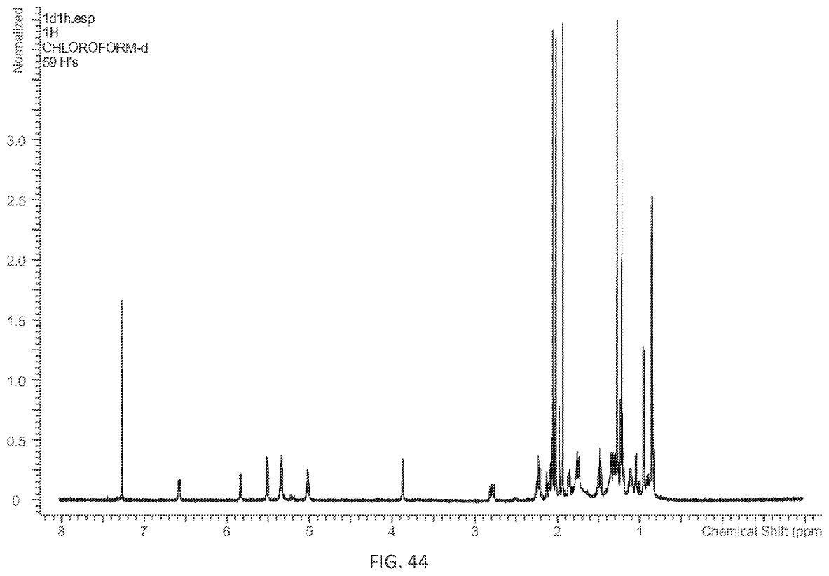

FIG. 45 is a graph illustrating a 13C NMR spectrum of alcyosterone (C5), 125 MHz, CDC₁₃.

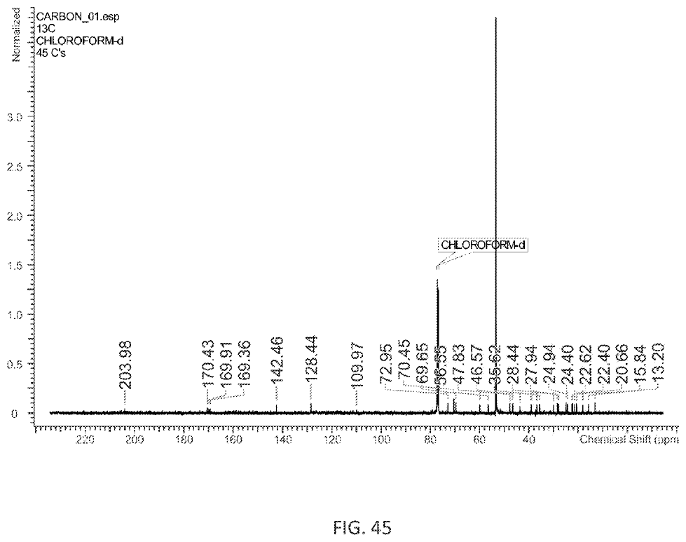

FIG. 46 is a graph illustrating a COSY spectrum of alcyosterone (C5), 500 MHz, CDC₁₃.

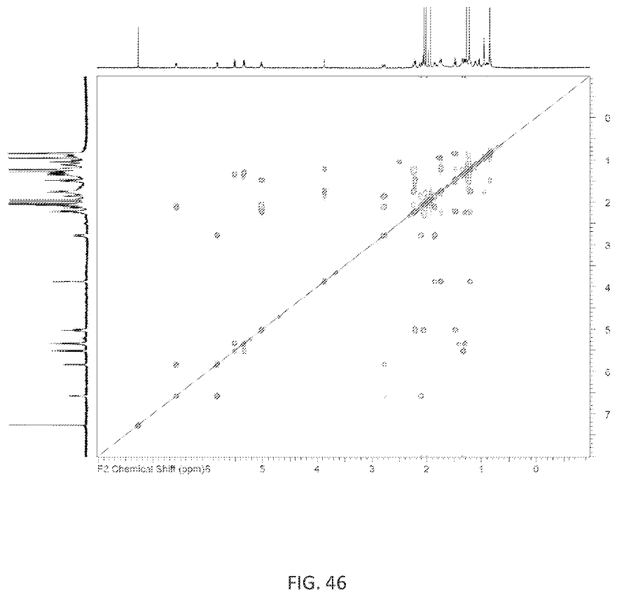

FIG. 47 is a graph illustrating an HSQC spectrum of alcyosterone (C5), 500 MHz, CDC₁₃.

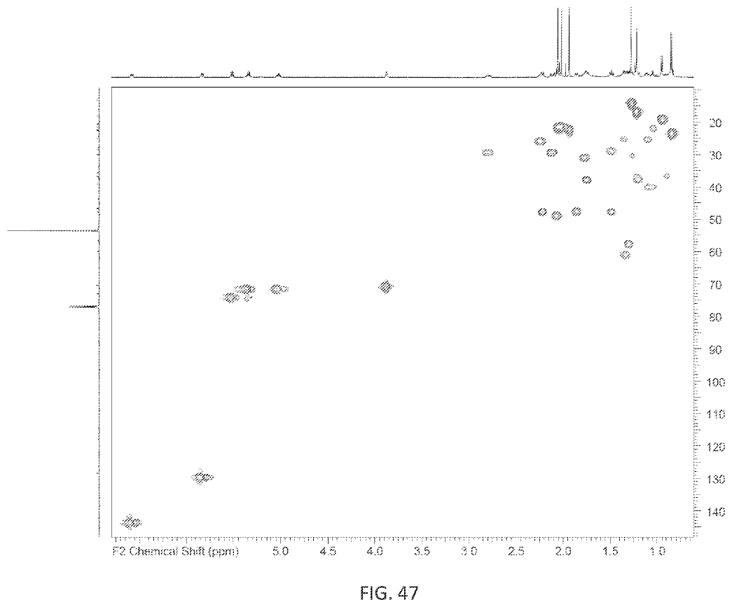

FIG. 48 is a graph illustrating an HMBC spectrum of alcyosterone (C5), 500 MHz, CDC₁₃.

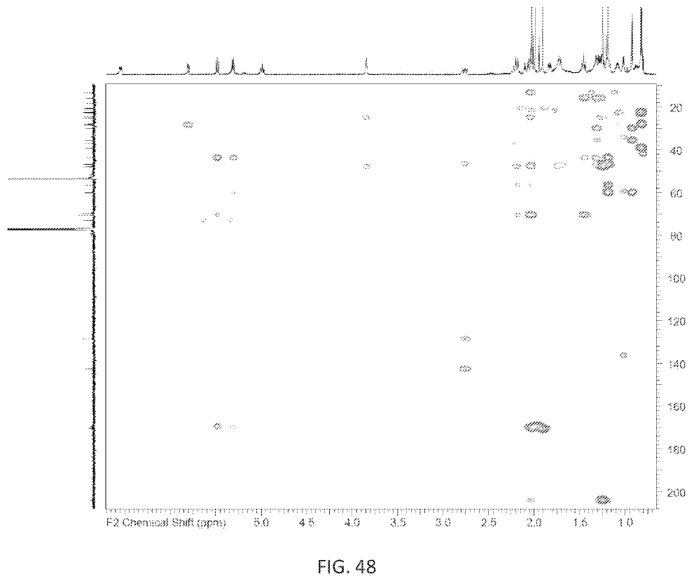

FIG. 49 is a graph illustrating HRESIMS of alcyosterone (C5). Calculated for C₃₃H₅₁O₈, m/z 575.3578 ([M+H]⁺); calculated for C31H47O6, m/z 515.3367 ([M−OAc]⁺); calculated for C₂₇H₃₉O₂, m/z 395.2945 ([M−OAc−2HOAc]⁺).

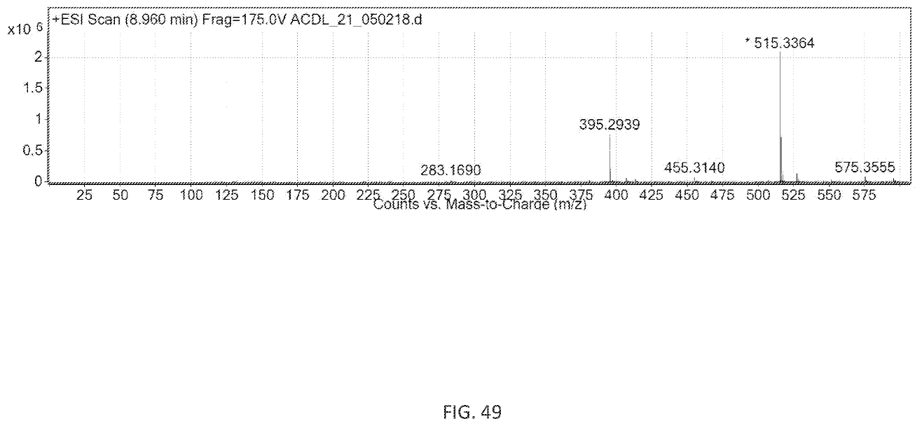

FIG. 50 is a drawing of an asymmetric unit of alcyosterone (C5). Anisotropic displacement parameters drawn at 50% probability level.

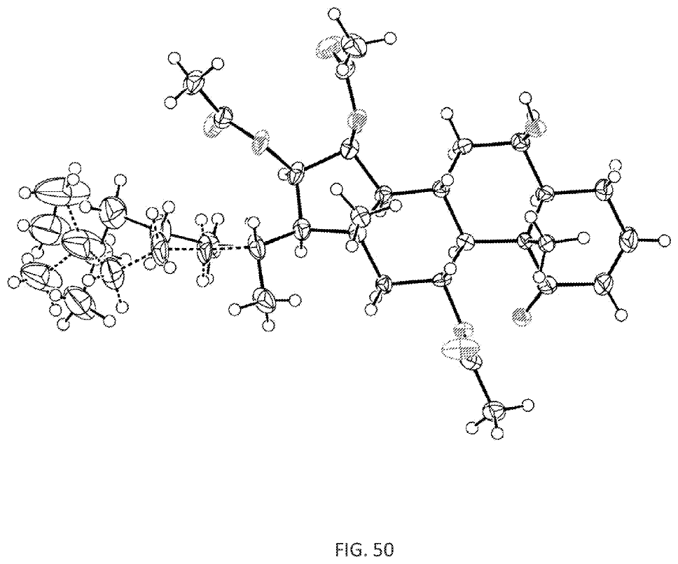

FIG. 51 is a drawing illustrating steps for a Soxhlet extraction protocol.

FIG. 52 is a drawing illustrating steps for dichloromethane/methanol (1:1) extraction protocol.

FIG. 53 illustrates an ¹H NMR spectrum of compound (C5), the elucidated structure of compound (5), and a reported activity of compound (C5) against Leishmaniasis donovani and ESKAPE pathogens.

FIG. 54 illustrates the activity of compounds (C3) and (C4) against various pathogens.

FIG. 55 illustrates an ¹H NMR spectrum and elucidated structure of compounds (C10).

FIG. 56 illustrates an ¹H NMR spectrum and elucidated structure of compounds (C11).

FIG. 57 is a graph illustrating the experimental circular dichroism variation for compound (C11).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

### Compounds P1-P8

Described herein are compounds and formulations thereof that can block sporozoite invasion and subsequent liver-stage parasite development. In some embodiments, the compound comprises one of compounds (P1) through (P8), derivatives thereof, or pharmaceutically acceptable salts thereof:

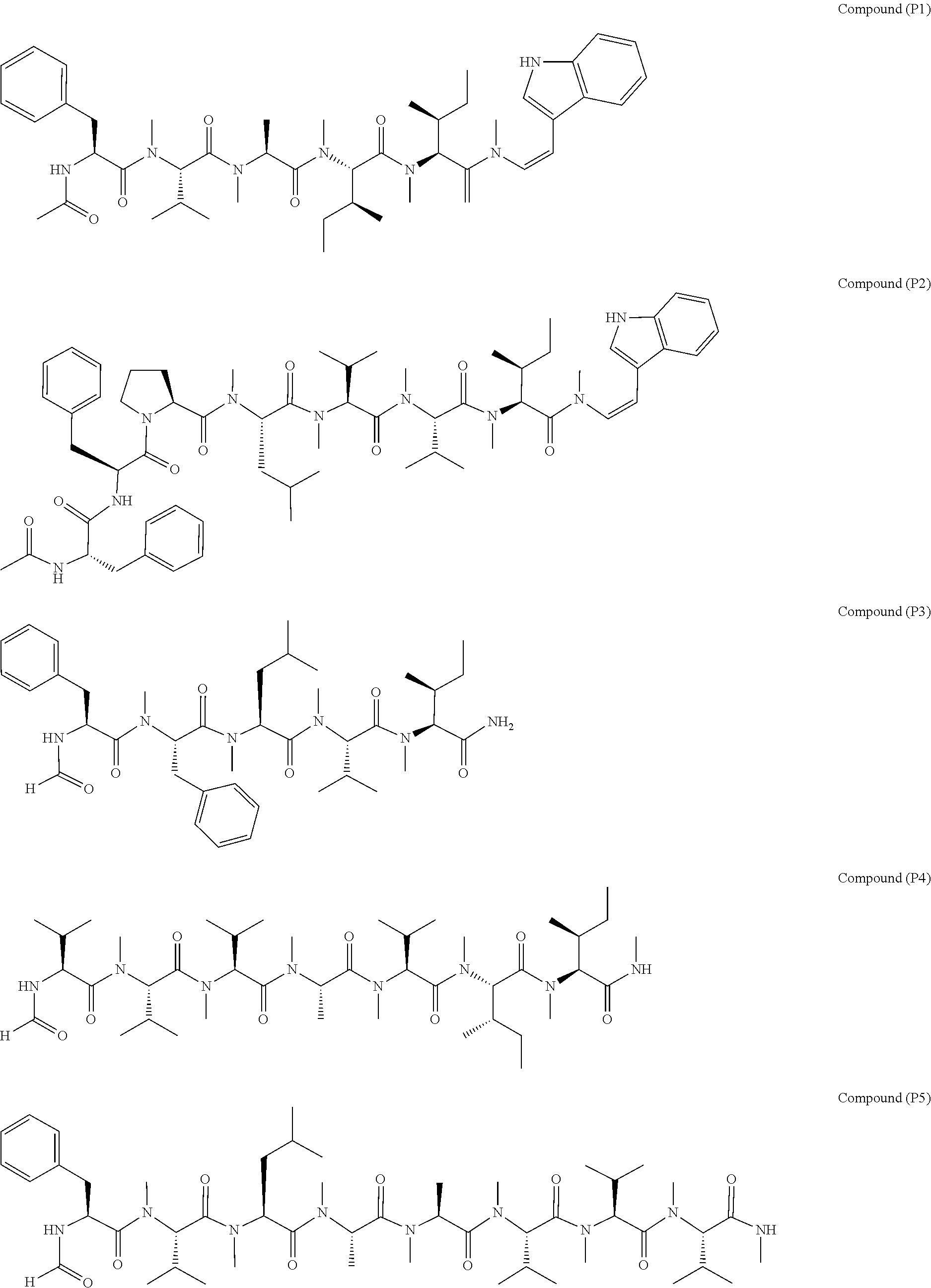

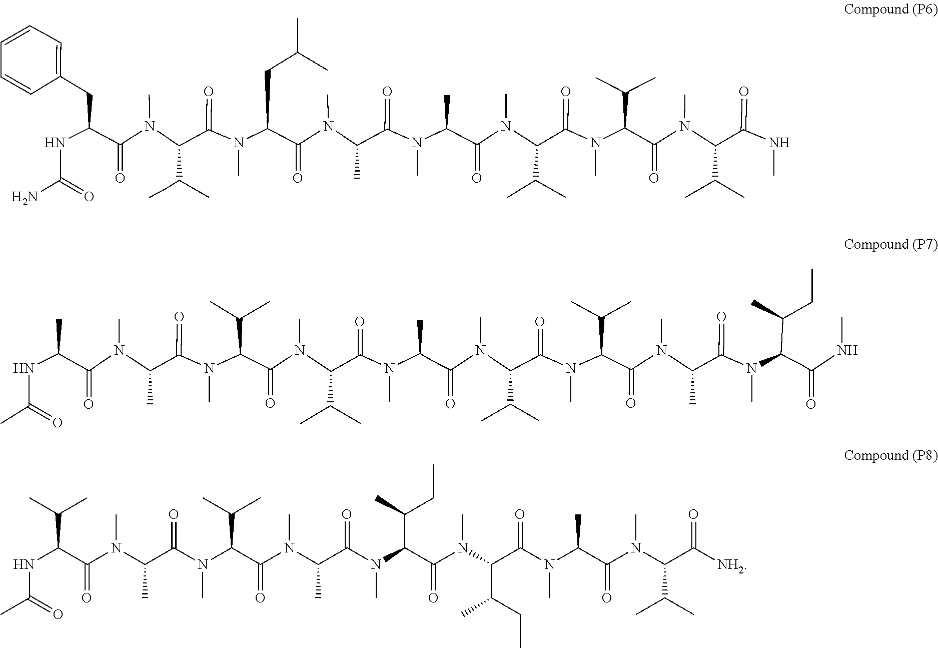

### Compounds C1-C9

Also described herein are compounds and formulations thereof that can block, inhibit, prevent, and/or treat bacterial and protist infections. In some embodiments, the compound can be any one of compounds (C1) through (C9), derivatives thereof, or pharmaceutically acceptable salts thereof.

Compounds C1, C2, C6, C7, and C8 are represented by the following structure (Formula I):
a.

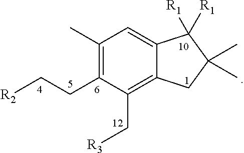

For compound (C1), also named alcyopterosin T, R₁═H, R₂=ONO₂, and R₃=OAc.

For compound (C2), also named alcyopterosin U, R₁═C-10 ketone, R₂=ONO₂, and R₃═OAc.

For compound (C6), also named alcyopterosin C, R₁═C-10 ketone, R₂=ONO₂, and R₃═H.

For compound (C7), also named alcyopterosin G, R₁═H, R₂=ONO₂, and R₃═OH.

For compound (C8), also named 4,12-Bis(acetyl)alcyopterosin O, R₁═H, R₂=OAc, and R₃═OAc.

Compounds C3, C4, and C9 are represented by the following structure (Formula II):
b.

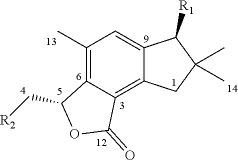

For compound (C3), also named alcyopterosin V, R₁═H, and R₂=OH.

For compound (C4), also named alcyopterosin E, R₁═H, and R₂=ONO₂.

For compound (C9), also named alcyopterosin L, R₁═OH, and R₂=CL Compound (C5), also named alcyosterone, is represented by the following structure:
c.

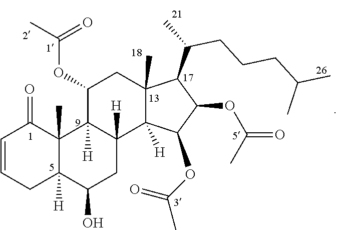

Compounds P1 through P8.

As detailed below, Compound (P1) is a stereoisomer of the compound friomaramide, disclosed in U.S. Pat. No. 11,331,367, filed on Dec. 23, 2019, which is incorporated by reference in its entirety.

Compounds (P1) through (P8) can be isolated from Antarctic sponge Inflatella coelosphaeroides according to the methods described in the Examples. After production, compounds (P1) through (P8) can be extracted and purified according to techniques generally known in the art and as described herein. Compounds (P1) through (P8), or pharmaceutically acceptable salts thereof, can also be synthesized using methods generally known in the art.

Compounds C1 through C9

As detailed below, compounds (C1) through (C9) can be isolated from Antarctic octocoral of the genus Alcyonium according to the methods described in the Examples. The compounds may also be synthesized. For example, compounds (C1) through (C9) may be synthesized through a multi-step synthesis scheme involving the reaction and/or addition of precursor compounds. In another example, the compounds may be synthesized through the modification of a similar or related base compound.

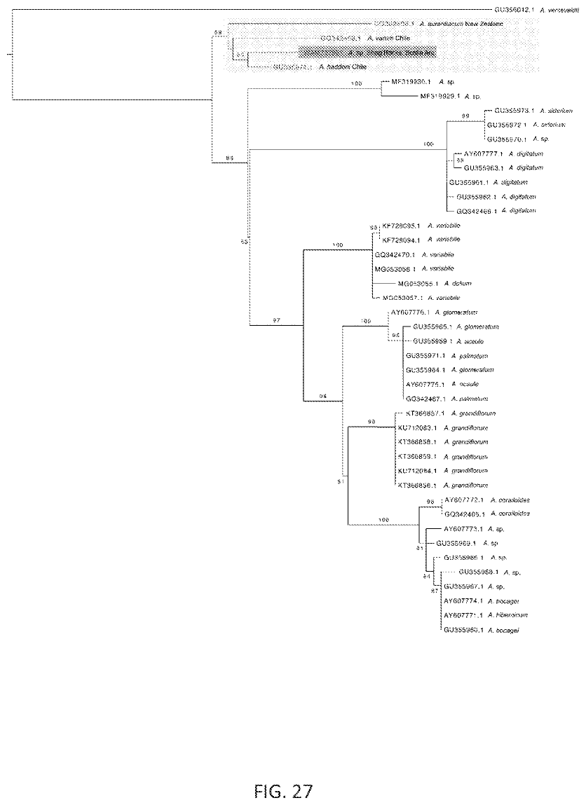

In one aspect, one of more of compounds (C1) through (C9) are derived from one or more species of the genus Alcyonium, such one of more species as shown in FIG. 27 including but not limited to A. verseldti, A. aurantiacum, A. varum, haddoni, A. siderium, A, digitatum, A. variable, A. dolium, A. glomeratum, A. acaule, A. palmatum, A. grandiflormur, A. colarlloides, A. bocagei, A. hibernicum, A. bocagei, Alcyonium sp. WAMZ 97931, or any strains thereof. One or more compounds of (C1) through (C9) may also be derived from a yet-unnamed species of Alcyonium.

### Compositions and Formulations Comprising Compounds P1-P8 and Compounds C1-C9

Compounds (P1) through (P8), derivatives thereof, or pharmaceutically acceptable salts thereof, and/or compounds (C1) through (C9), derivatives thereof, or pharmaceutically acceptable salts thereof, can be included in formulations that, in addition to the compounds, can further include a suitable carrier. The carrier can be a pharmaceutically acceptable carrier. The formulation can be a pharmaceutical formulation. The compounds, salts and/or formulations thereof described herein can be administered to a subject. The subject can be infected with or be suspected of being infected with a leishmanial and/or plasmodium parasite. The subject so infected, or suspected of infection, can be considered a subject in need thereof. The compounds and formulations described herein can be administered by a suitable route, such as but not limited to oral, topical (e.g., by cream, solution, or patch), and parenteral. Exemplary suitable routes are described elsewhere herein.

Compounds (P1) through (P8), derivatives thereof, or pharmaceutically acceptable salts thereof, and formulations thereof, and/or Compounds (C1) through (C9), derivatives thereof, or pharmaceutically acceptable salts thereof, and formulations thereof described herein can be formulated for parenteral delivery, such as injection or infusion, in the form of a solution or suspension. The formulation can be administered via any route, such as the blood stream, or directly to the organ or tissue to be treated.

Parenteral formulations can be prepared as aqueous compositions using techniques known in the art. Typically, such compositions can be prepared as injectable formulations, for example, solutions or suspensions; solid forms suitable for preparing solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

For all compounds and formulations herein, a carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

For all compounds and formulations herein, solutions and dispersions of the compounds (P1) through (P8), pharmaceutically acceptable salts thereof, or formulations thereof and/or Compounds (C1) through (C9), derivatives thereof, or pharmaceutically acceptable salts thereof, and formulations thereof described herein can be prepared in water or another solvent or dispersing medium suitably mixed with one or more pharmaceutically acceptable excipients including, but not limited to, surfactants, dispersants, emulsifiers, pH modifying agents, and combination thereof.

As used herein, suitable surfactants can be anionic, cationic, amphoteric or nonionic surface-active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-p-alanine, sodium N-lauryl-β-iminodipropionate, myristoamphoacetate, lauryl betaine and lauryl sulfobetaine.

The formulations can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of Compounds (P1) through (P8) and/or Compounds (C1) through (C9).

For all formulations herein, the formulations can be buffered to a pH of 3-8 for parenteral administration upon reconstitution. In some aspects, the pH of the formulations can be a pH of about 7.0-7.4 upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating one or more of Compounds (P1) through (P8) derivatives thereof, or a pharmaceutically acceptable salt thereof and/or one or more of Compounds (C1) through (C9), derivatives thereof, or pharmaceutically acceptable salts thereof in the desired amount in the appropriate solvent or dispersion medium with one or more of the excipients listed above, as required, followed by filtered sterilization. Dispersions can be prepared by incorporating one or more Compounds (P1) through (P8) derivatives thereof, or a pharmaceutically acceptable salt thereof and/or one or more of Compounds (C1) through (C9), derivatives thereof, or pharmaceutically acceptable salts thereof into a sterile vehicle that contains the basic dispersion medium and the required other ingredients from those listed above. Sterile powders for the preparation of sterile injectable solutions can be prepared by vacuum-drying and freeze-drying techniques, which yields a powder of one or more of Compounds (P1) through (P8) derivatives thereof, or a pharmaceutically acceptable salt thereof and/or one or more of Compounds (C1) through (C9), derivatives thereof, or pharmaceutically acceptable salts thereof with or without any additional desired ingredient from a previously sterile-filtered solution thereof. The powders can be prepared in such a manner that the particles are porous in nature, which can increase dissolution of the particles. Methods for making porous particles are well-known in the art.

Pharmaceutical formulations for parenteral administration can be in the form of a sterile aqueous solution or suspension of one or more of Compounds (P1) through (P8), derivatives thereof, or pharmaceutically acceptable salts thereof and/or one or more of Compounds (C1) through (C9), derivatives thereof, or pharmaceutically acceptable salts thereof. Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulations disclosed herein can be distributed or packaged in a liquid form. In other embodiments, formulations for parenteral administration can be packed as a solid, obtained, for example, by lyophilization of a suitable liquid formulation. The solid can be reconstituted with an appropriate carrier or diluent prior to administration.

Solutions, suspensions, or emulsions for parenteral administration disclosed herein can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration disclosed herein can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration disclosed herein can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions disclosed herein, and use of nanotechnology including nanoformulations for parenteral administration disclosed herein can also contain one or more excipients, such as dispersing agents, wetting agents, and suspending agents.

One or more of Compounds (P1) through (P8), derivatives thereof, or pharmaceutically acceptable salts thereof and/or one or more of Compounds (C1) through (C9), derivatives thereof, or pharmaceutically acceptable salts thereof can be formulated for topical administration. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some embodiments, one or more of Compounds (P1) through (P8), derivatives above, or pharmaceutically acceptable salts thereof and/or one or more of Compounds (C1) through (C9), derivatives thereof, or pharmaceutically acceptable salts thereof, can be administered as a liquid formulation, such as a solution or suspension, a semi-solid formulation, such as a lotion or ointment, or a solid formulation. In some embodiments, one or more of Compounds (P1) through (P8), or pharmaceutically acceptable salts thereof, and/or one or more of Compounds (C1) through (C9), derivatives thereof, or pharmaceutically acceptable salts thereof can be formulated as liquids, including solutions and suspensions, such as eye drops or as a semi-solid formulation, such as ointment or lotion for topical application to the skin, to the mucosa, such as the eye, to the vagina, or to the rectum.

The formulations disclosed herein can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some embodiments, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some embodiments, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water and water-in-oil emulsions. Either or both phases of the emulsions can include a surfactant, an emulsifying agent, and/or a liquid non-volatile non-aqueous material. In some embodiments, the surfactant can be a non-ionic surfactant. In other embodiments, the emulsifying agent is an emulsifying wax. In further embodiments, the liquid non-volatile non-aqueous material is a glycol. In some embodiments, the glycol is propylene glycol. The oil phase can contain other suitable oily pharmaceutically acceptable excipients. Suitable oily pharmaceutically acceptable excipients include, but are not limited to, hydroxylated castor oil and sesame oil. These excipients can be used in the oil phase as surfactants or emulsifiers.

Lotions containing one or more of Compounds (P1) through (P8), or pharmaceutically acceptable salts thereof, and/or one or more of Compounds (C1) through (C9), derivatives thereof, or pharmaceutically acceptable salts thereof are also described herein. In some embodiments, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing one or more of Compounds (P1) through (P8), or pharmaceutically acceptable salts thereof, and/or one or more of Compounds (C1) through (C9), derivatives thereof, or pharmaceutically acceptable salts thereof are also described herein. The cream can contain emulsifying agents and/or other stabilizing agents. In some embodiments, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations. Creams can be thicker than lotions, can have various uses, and can have more varied oils/butters, depending upon the desired effect upon the skin. In some embodiments of a cream formulation, the water-base percentage can be about 60% to about 75% and the oil-base can be about 20% to about 30% of the total, with the other percentages being the emulsifier agent, preservatives and additives for a total of 100%.

Ointments containing one or more of Compounds (P1) through (P8), or pharmaceutically acceptable salts thereof, and/or one or more of Compounds (C1) through (C9), derivatives thereof, or pharmaceutically acceptable salts thereof and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy than ointments prepared with the same components.

Also described herein are gels containing one or more of Compound (P1) through (P8), or pharmaceutically acceptable salts thereof and/or one or more of Compounds (C1) through (C9), derivatives thereof, or pharmaceutically acceptable salts thereof, a gelling agent, and a liquid vehicle. Suitable gelling agents include, but are not limited to, modified celluloses, such as hydroxypropyl cellulose and hydroxyethyl cellulose; carbopol homopolymers and copolymers; thermoreversible gels and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited to, isopropyl myristate, ethyl acetate, C12-C15 alkyl benzoates, mineral oil, squalane, cyclomethicone, capric/caprylic triglycerides, and combinations thereof.

Also described herein are foams that can include one or more of Compounds (P1) through (P8), derivatives thereof, or pharmaceutically acceptable salts thereof, or one or more of Compounds (C1) through (C9), derivatives thereof, or pharmaceutically acceptable salts thereof. Foams can be an emulsion in combination with a gaseous propellant. The gaseous propellant can include hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or can become approved for medical use are suitable. The propellants can be devoid of hydrocarbon propellant gases, which can produce flammable or explosive vapors during spraying. Furthermore, the foams can contain no volatile alcohols, which can produce flammable or explosive vapors during use.

Buffers can be used to control pH of compositions disclosed herein. The buffers can buffer the composition from a pH of about 4 to a pH of about 7.5, from a pH of about 4 to a pH of about 7, or from a pH of about 5 to a pH of about 7. In some embodiments, the buffer can be triethanolamine.

Preservatives can be included in formulations disclosed herein to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, the formulations disclosed herein can be provided via continuous delivery of one or more formulations to a patient in need thereof. For topical applications, repeated applications can be performed, or a patch can be used to provide continuous administration of the noscapine analogs over an extended period of time.

One or more of Compounds (P1) through (P8), derivatives thereof, or pharmaceutically acceptable salts thereof, and/or one or more of Compounds (C1) through (C9), derivatives thereof, or pharmaceutically acceptable salts thereof can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations containing one or more of Compounds (P1) through (P8), derivatives thereof, or pharmaceutically acceptable salts thereof, and/or one or more of Compounds (C1) through (C9), derivatives thereof, or pharmaceutically acceptable salts thereof, can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition which can include plasticizers, pigments, colorants, stabilizing agents, and glidants.

Formulations containing one or more of Compounds (P1) through (P8), derivatives thereof, 5 or pharmaceutically acceptable salts thereof, and/or one or more of Compounds (C1) through (C9), derivatives thereof, or pharmaceutically acceptable salts thereof, can be prepared using one or more pharmaceutically acceptable excipients, including diluents, preservatives, binders, lubricants, disintegrators, swelling agents, fillers, stabilizers, and combinations thereof.

Delayed release dosage formulations containing one or more of Compounds (P1) through (P8), derivatives thereof, or pharmaceutically acceptable salts thereof, and/or one or more of Compounds (C1) through (C9), derivatives thereof, or pharmaceutically acceptable salts thereof can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and processes for preparing tablets and capsules and delayed release dosage forms of tablets, capsules, and granules. These references provide information on carriers, materials, equipment and processes for preparing tablets and capsules and delayed release dosage forms of tablets, capsules, and granules.

The formulations containing one or more of Compounds (P1) through (P8), derivatives thereof, or pharmaceutically acceptable salts thereof, and/or one or more of Compounds (C1) through (C9), derivatives thereof, or pharmaceutically acceptable salts thereof can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water-soluble polymer, water insoluble polymers and/or pH dependent polymers, with or without water-insoluble/water-soluble non polymeric excipient, to produce the desired release profile. The coating can be performed on a dosage form (matrix or simple) that includes, but is not limited to, tablets (compressed with or without coated beads), capsules (with or without coated beads), beads, particle compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers, and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, and powdered sugar. The usual diluents include inert powdered substances such as starches, powdered cellulose, especially crystalline and microcrystalline cellulose, sugars such as fructose, mannitol, sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can be used herein to impart cohesive qualities to a solid dosage formulation, and thus can ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid, and hydrogenated vegetable oils.

Disintegrants can be used herein to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross-linked polymers, such as cross-linked PVP (Polyplasdone® XL from GAF Chemical Corp).

Stabilizers can be used herein to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

In use, one or more of Compounds (P1) through (P8), derivatives thereof, or pharmaceutically acceptable salts thereof, and/or one or more of Compounds (C1) through (C9), derivatives thereof, or pharmaceutically acceptable salts thereof can be administered to a subject. In some embodiments, the subject is infected with or is suspected of being infected with a protozoan parasite, such as Plasmodium falciparum. One or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof described herein can be co-administered or be a co-therapy with another active agent or ingredient (e.g., an antimalarial drug such as primaquine) that can be included in the formulation or provided in a dosage form separate from the Compound (P1), a pharmaceutically acceptable salt thereof, or formulation thereof.

The amount of one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can range from about 0.01 μg/kg to up to about 1000 mg/kg or more, depending on the factors mentioned elsewhere herein. In certain embodiments, the amount can range from 0.01 μg/kg up to about 500 mg/kg, or 1 μg/kg up to about 500 mg/kg, 5 μg/kg up to about 500 mg/kg, 0.01 μg/kg up to about 100 mg/kg, or 1 μg/kg up to about 100 mg/kg, 5 μg/kg up to about 100 mg/kg.

Administration of one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be systemic or localized. One or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered to the subject in need thereof one or more times per hour or day. In embodiments, one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered once daily. In other embodiments, Compound (P1), a pharmaceutically acceptable salt thereof, or formulation thereof can be administered 1 (q.d.), 2 (b.i.d.), 3 (t.i.d), 4 (q.i.d.), or more times daily. In some embodiments, when administered, an effective amount of one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered to the subject in need thereof. One or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered one or more times per week. In some embodiments, one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered 1, 2, 3, 4, 5, 6 or 7 days per week. In some embodiments, one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered 1, 2, 3, 4, 5, 6, 7, 8, 9, 10, 11, 12, or more times per month. In some embodiments, one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered 1, 2, 3, 4, 5, 6, 7, 8, 9, 10, 11, 12, 13, 14, 15, 16, 17, 18, or more times per year.

In some embodiments, one or more of Compounds (P1) through (P8), derivatives thereof, pharmaceutically acceptable salts thereof, or formulations thereof, and/or one or more of Compounds (C1) through (C9), derivatives thereof, or pharmaceutically acceptable salts thereof, or formulations thereof can be administered in a dosage form. The amount or effective amount of Compounds (P1) through (P8), derivatives thereof, pharmaceutically acceptable salts thereof, or formulations thereof, and/or Compounds (C1) through (C9), derivatives thereof, or pharmaceutically acceptable salts thereof, or formulations thereof can be divided into multiple dosage forms. For example, the effective amount can be split into two dosage forms with one one dosage forms administered, for example, in the morning, and the second dosage form administered in the evening. Although the effective amount can be given over two or more doses, in one day, the subject can receive the effective amount when the total amount administered across all the doses is considered. The dosages can range from about 0.01 μg/kg to up to about 1000 mg/kg or more, depending on the factors mentioned above. In certain embodiments, the dosage can range from 0.01 μg/kg up to about 500 mg/kg, or 1 μg/kg up to about 500 mg/kg, 5 μg/kg up to about 500 mg/kg, 0.01 μg/kg up to about 100 mg/kg, or 1 μg/kg up to about 100 mg/kg, 5 μg/kg up to about 100 mg/kg.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Compounds (P1) through (P8), derivatives thereof, pharmaceutically acceptable salts thereof, and/or Compounds (C1) through (C9), derivatives thereof, or pharmaceutically acceptable salts thereof can be included in formulations, that, in addition to the compounds, can further include a suitable carrier. The carrier can be a pharmaceutically acceptable carrier. The formulation can be a pharmaceutical formulation. The compounds, salts and/or formulations thereof described herein can be administered to a subject. The subject can be infected with or be suspected of being infected with a parasite, such as a leishmanial and/or plasmodium parasite, or a bacterium, such as Clostridium difficile.

The subject so infected, or suspected of infection, can be considered a subject in need thereof. The compounds and formulations described herein can be administered by a suitable route, such as but not limited to oral, topical (e.g., by cream, solution, or patch), and parenteral. Exemplary suitable routes are described elsewhere herein.

In use, one or more of Compounds (P1) through (P8), derivatives thereof, pharmaceutically acceptable salts thereof, and/or one or more of Compounds (C1) through (C9), derivatives thereof, or pharmaceutically acceptable salts thereof (e.g., compositions comprising one or more of Compounds (C1) through (C9) and/or (P1) through (P8)) described herein can be administered to a subject. In some embodiments, the subject is infected with or is suspected of being infected with a protozoan parasite, such as Leishmania donovani. One or more of Compounds (P1) through (P8), derivatives thereof, pharmaceutically acceptable salts thereof, and/or one or more of Compounds (C1) through (C9), derivatives thereof, or pharmaceutically acceptable salts thereof described herein can be co-administered or be a co-therapy with another active agent or ingredient (e.g., an antileishmanial drug such as amphotericin B) that can be included in the formulation or provided in a dosage form separate from the Compounds (P1) through (P8), derivatives thereof, pharmaceutically acceptable salts thereof, and/or Compounds (C1) through (C9), derivatives thereof, or pharmaceutically acceptable salts thereof.

The amount of one or more of Compounds (P1) through (P8), derivatives thereof, pharmaceutically acceptable salts thereof, and/or one or more of Compounds (C1) through (C9), derivatives thereof, or pharmaceutically acceptable salts thereof thereof can range from about 0.01 μg/kg to up to about 1000 mg/kg or more, depending on the factors mentioned elsewhere herein. In certain embodiments, the amount can range from 0.01 μg/kg up to about 500 mg/kg, or 1 μg/kg up to about 500 mg/kg, 5 μg/kg up to about 500 mg/kg, 0.01 μg/kg up to about 100 mg/kg, or 1 μg/kg up to about 100 mg/kg, 5 μg/kg up to about 100 mg/kg.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

As used in the specification and the appended claims, the singular forms “a,” “an” and “the” include plural referents unless the context clearly dictates otherwise.

As used herein, the terms “optional” or “optionally” means that the subsequently described event or circumstance can or cannot occur, and that the description includes instances where said event or circumstance occurs and instances where it does not.

As used herein, “administering” refers to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intraarticular, intra-synovial, intrasternal, intrathecal, intrahepatic, intralesional, and intracranial injections or infusion techniques.

Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “agent” refers to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “dose,” “unit dose,” or “dosage” refers to physically discrete units suitable for use in a subject, each unit containing a predetermined quantity of Compound (P1) and/or a formulation thereof calculated to produce the desired response or responses in association with its administration.

As used herein, “effective amount” refers to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

The term “subject” refers to any individual who is the target of administration or treatment.

The subject can be a vertebrate, for example, a mammal. Thus, the subject can be a human or veterinary patient. The term “patient” refers to a subject under the treatment of a clinician, e.g., physician.

As used herein, “substantially pure” means an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” refers to an amount (e.g., mass, volume, dosage, concentration, and/or time period) needed to achieve one or more desired result(s). For example, a therapeutically effective amount refers to an amount needed to achieve one or more therapeutic effects. In some embodiments, the effective amount can be anti-parasitic. In some embodiments, the effective amount can kill and/or inhibit a leishmanial parasite. In some embodiments, the effective amount can treat a leishmanial infection in a subject.

In some embodiments the disclosure relates to any of the following numbered paragraphs:

### EXEMPLARY EMBODIMENTS

1. A method of treating infection of a protozoan parasite in a subject in need thereof, the method including: administering to the subject an effective amount of a composition comprising a compound, wherein the compound is selected from the group consisting of:

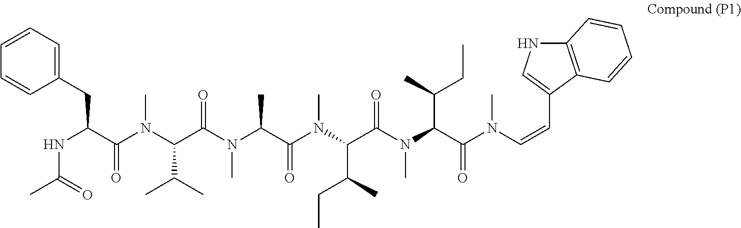 [SMILES: CC[C@H](C)[C@@H](C(=O)N(C)/C=C\c1c[nH]c2ccccc12)N(C)C(=O)[C@H]([C@@H](C)CC)N(C)C(=O)[C@H](C)N(C)C(=O)[C@H](C(C)C)N(C)C(=O)[C@H](Cc1ccccc1)NC(C)=O]

or a pharmaceutically acceptable salt thereof, wherein the configuration of the phenylalanyl group is L;

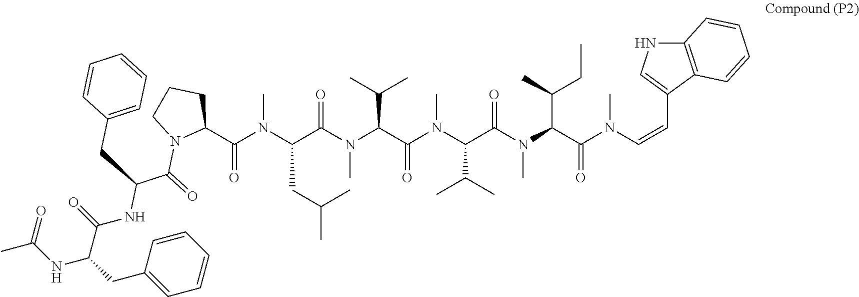 [SMILES: CC[C@H](C)[C@@H](C(=O)N(C)/C=C\c1c[nH]c2ccccc12)N(C)C(=O)[C@H](C(C)C)N(C)C(=O)[C@H](C(C)C)N(C)C(=O)[C@H](CC(C)C)N(C)C(=O)[C@@H]1CCCN1C(=O)[C@H](Cc1ccccc1)NC(=O)[C@H](Cc1ccccc1)NC(C)=O]

or a pharmaceutically acceptable salt thereof;

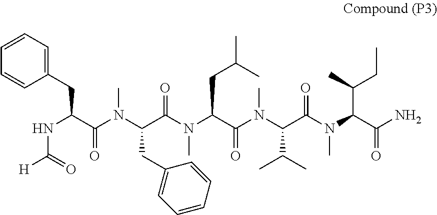 [SMILES: CC[C@H](C)[C@@H](C(N)=O)N(C)C(=O)[C@H](C(C)C)N(C)C(=O)[C@H](CC(C)C)N(C)C(=O)[C@H](Cc1ccccc1)N(C)C(=O)[C@H](Cc1ccccc1)NC=O]

or a pharmaceutically acceptable salt thereof;

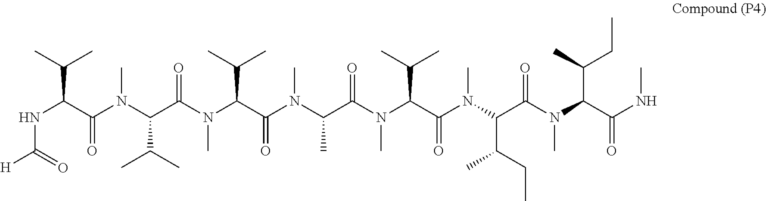

or a pharmaceutically acceptable salt thereof;

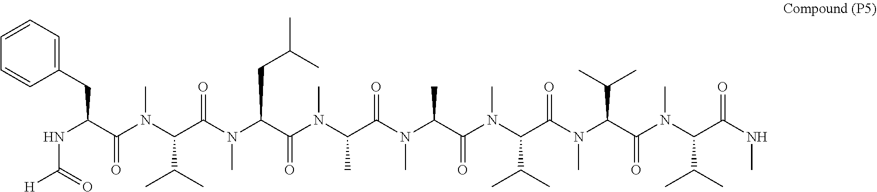 [SMILES: CNC(=O)[C@H](C(C)C)N(C)C(=O)[C@H](C(C)C)N(C)C(=O)[C@H](C(C)C)N(C)C(=O)[C@H](C)N(C)C(=O)[C@H](C)N(C)C(=O)[C@H](CC(C)C)N(C)C(=O)[C@H](C(C)C)N(C)C(=O)[C@H](Cc1ccccc1)NC=O]

or a pharmaceutically acceptable salt thereof;

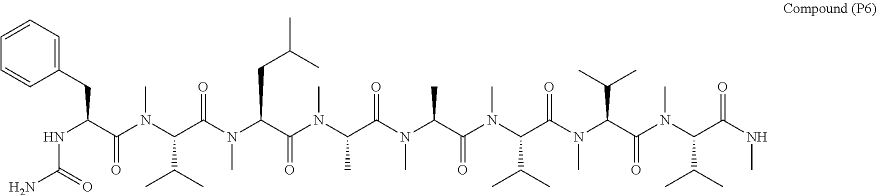 [SMILES: CNC(=O)[C@H](C(C)C)N(C)C(=O)[C@H](C(C)C)N(C)C(=O)[C@H](C(C)C)N(C)C(=O)[C@H](C)N(C)C(=O)[C@H](C)N(C)C(=O)[C@H](CC(C)C)N(C)C(=O)[C@H](C(C)C)N(C)C(=O)[C@H](Cc1ccccc1)NC(N)=O]

or a pharmaceutically acceptable salt thereof;

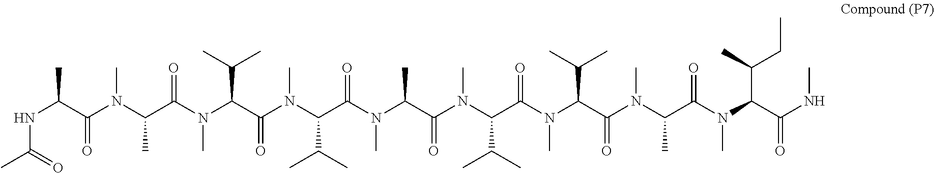 [SMILES: CC[C@H](C)[C@@H](C(=O)NC)N(C)C(=O)[C@H](C)N(C)C(=O)[C@H](C(C)C)N(C)C(=O)[C@H](C(C)C)N(C)C(=O)[C@H](C)N(C)C(=O)[C@H](C(C)C)N(C)C(=O)[C@H](C(C)C)N(C)C(=O)[C@H](C)N(C)C(=O)[C@H](C)NC(C)=O]

or a pharmaceutically acceptable salt thereof; and

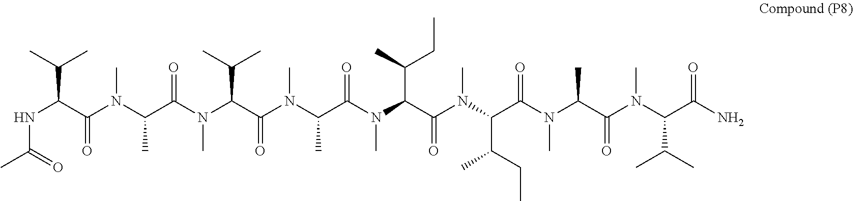 [SMILES: CC[C@H](C)[C@@H](C(=O)N(C)[C@@H](C)C(=O)N(C)[C@H](C(N)=O)C(C)C)N(C)C(=O)[C@H]([C@@H](C)CC)N(C)C(=O)[C@H](C)N(C)C(=O)[C@H](C(C)C)N(C)C(=O)[C@H](C)N(C)C(=O)[C@@H](NC(C)=O)C(C)C]

or a pharmaceutically acceptable salt thereof.
2. The method of paragraph 1, wherein the compound consists of Compound (P1) or a pharmaceutically acceptable salt thereof.
3. The method of paragraph 1, wherein the compound consists of Compound (P2) or a pharmaceutically acceptable salt thereof.
4. The method of paragraph 1, wherein the compound consists of Compound (P3) or a pharmaceutically acceptable salt thereof.
5. The method of paragraph 1, wherein the compound consists of Compound (P4) or a pharmaceutically acceptable salt thereof.
6. The method of paragraph 1, wherein the compound consists of Compound (P5) or a pharmaceutically acceptable salt thereof.
7. The method of paragraph 1, wherein the compound consists of Compound (P6) or a pharmaceutically acceptable salt thereof.
8. The method of paragraph 1, wherein the compound consists of Compound (P7) or a pharmaceutically acceptable salt thereof.
9. The method of paragraph 1, wherein the compound consists of Compound (P8) or a pharmaceutically acceptable salt thereof.
10. The method of any one of paragraph 1-9, wherein the protozoan parasite is Plasmodium falciparum.
11. The method of any one of paragraph 1-10, wherein the protozoan parasite is a drug resistant strain of Plasmodium falciparum.
12. The method of paragraph 11, wherein the drug resistant strain of Plasmodium falciparum: is resistant to artemisinin.
13. The method of any one of paragraph 1-12, wherein the composition is isolated from Antarctic sponge Inflatella coelosphaeroides.
14. The method of any one of paragraph 1-13, further comprising administering to the subject an effective amount of an antimalarial drug.
15. The method of paragraph 14, wherein the antimalarial drug comprises primaquine.
16. A method of treating infection of a bacterium or a protist in a subject in need thereof, the method including: administering to the subject an effective amount of a composition comprising a compound, wherein the compound comprises or is selected from a group consisting of:
(a) compounds having a structure of formula I

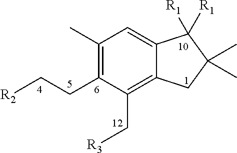

or a pharmaceutically acceptable salt thereof,
wherein R₁═H or a C-10 alkyl comprising a ketone,
wherein R₂=ONO₂ or OAc, and
wherein R₃═H, OAc, or OH;
(b) compounds having a structure of formula II

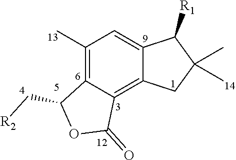

or a pharmaceutically acceptable salt thereof,
wherein R₁═H or OH, and
wherein R₂=OH, ONO₂, or C1; or
(c) alcyosterone

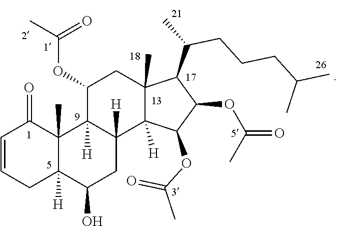

or a pharmaceutically acceptable salt thereof, or a combination thereof.
17. The method of paragraph 16, wherein the compound in the composition consists of the compound having the structure of formula I or the pharmaceutically acceptable salt thereof.
18. The method of paragraph 16, wherein the compound in the composition consists of the compound having the structure of formula II or a pharmaceutically acceptable salt thereof.
19. The method of paragraph 16, wherein the compound in the composition consists of alcyosterone or a pharmaceutically acceptable salt thereof.
20. The method of paragraph m 17, wherein R₁═H, R₂═ONO₂, and R₃═OAC.
21. The method of paragraph 17, wherein R₁═C-10 alkyl comprising a ketone, R₂═ONO₂ and R₃═OAC.
22. The method of paragraph 17, wherein R₁═C-10 alkyl comprising a ketone, R₂═ONO₂ and R₃═H.
23. The method of paragraph 17, wherein R₁═H, R₂═ONO₂ and R₃═OH.
24. The method of paragraph aim 17, wherein R₁═H, R₂═OAc and R₃═OAc.
25. The method of paragraph 18, wherein Rt=H, and R₂═OH.
26. The method of paragraph 18, wherein Rt=H, and R₂═ONO₂.
27. The method of paragraph 18, wherein Rt=OH, and R₂═Cl.
28. The method of any one of paragraph 16-27, wherein the infection includes a bacterial infection.
29. The method of paragraph 28, wherein the bacteria is Clostridium difficile.
30. The method of any one of paragraph 16-27, wherein the infection comprises a protist.
31. The method of paragraph 30, wherein the protist is Leishmania donovani.
32. The method of any one of paragraph 16-31, wherein the compound is isolated from Alcyonium sp.
33. The method of paragraph 32, wherein the Alcyonium sp. is an Antarctic coral.
34. The method of any one of paragraph 16-33, comprising an additional active agent.
35. The method of paragraph 34, wherein the additional active agent comprises one or more of an antibiotic, and an anti-inflammatory agent.

### EXAMPLES

The following examples are illustrative and should not be interpreted to limit the scope of the claimed subject matter.

### Example 1

Purification of Eight Novel Compounds from Inflatella Coelosphaeroides.

Malaria represents a serious threat to the health of a large proportion of the world population. Despite worldwide effort to control the disease, infections continue to increase annually with 241 million cases and 627,000 deaths worldwide in 2020.¹ The parasite Plasmodium falciparum: is one of the most common sources of malaria, and although several treatments exist, strains resistant to widely used drugs are spreading throughout the world.² Recently it has been observed that artemisinin combination therapy, which has been the gold standard for malaria treatment, is now starting to fail in some African countries.³⁻⁴ What's more concerning is that strain resistance appears to be independent of the resistance parasites strains observed in southeast Asia where artemisinin was first discovered and used.⁴ Africa independently accounts for more than 90% of malaria deaths worldwide and there is a pressing need for completely new drugs and treatment options.⁵

Natural products are a proven source of anti-malarial metabolites, with the plant sourced quinine and artemisinin possibly the most widely appreciated to date.⁶ Beyond terrestrial plants, marine organisms have also been a fruitful resource too,⁷ with marine sponges leading to at least 259 unique metabolites (as of 2019) that have demonstrated anti-Plasmodium falciparum activity.⁸

Extreme environmental habitats of polar regions such as that of the Southern Ocean around Antarctica, generate considerable chemical diversity associated with invertebrate inhabitants.⁹⁻¹¹ The intense competition for scarce resources results in a need for sedentary invertebrates to acquire bioactive secondary metabolites, in order to increase chances of survival by mitigating predation, fouling and other forms of special competition.¹² Annually, sponges and their biosynthetically gifted microorganisms, continue to be one of the most fruitful sources for the discovery of new marine natural products,¹³ including those collected from Antarctic waters.¹⁴⁻¹⁵ Numerous novel peptidic natural products believed to be produced by NRPS and RIPP biosynthetic pathways have been discovered from these marine invertebrates, such as linear N-methylated peptides.¹⁶⁻¹⁸ RHM1 and RHM2 are highly N-methylated octapeptides isolated from a sponge-derived Acremonium sp. that showed mild cytotoxic and antibacterial activity,¹⁷ while the octapeptide pembamide isolated from Cribrochalina sp. showed cytotoxicity against human tumor cell lines.¹⁶ The substitution of canonical amino acids for N-methylated congeners has been shown to alter biological functions of polypeptides in desirable ways for the purpose of drug discovery and development, such as improved stability, binding, and oral bioavailability.¹⁹⁻²⁰ Our previous investigation of the sponge Inflate/la coelosphaeroides following an in vitro liver stage malaria screen, led to the isolation of friomaramide (P1), a highly modified hexapeptide with a permethylated amide backbone and a tryptenamine residue at the C-terminus.²¹ Friomaramide demonstrated strong anti-malarial potential by blocking the liver stage of Plasmodium falciparum parasite development at levels comparable to the known drug primaquine.

As part of our continuing investigation into the chemistry of Antarctic marine invertebrates, we turned our attention back to the sponge Inflatella coelosphaeroides. Spectroscopic analysis of the unanalyzed I. coelosphaeroides extract fractions, along with the methanolic extract of a previously unexplored specimen, showed many signals indicative of other N-methylated peptides, and were therefore subjected to a 1H NMR and MS-guided isolation procedure. Through this, seven previously unreported N-methylated peptides were isolated, and their structures solved by a combination of NMR and MS/MS fragmentation analysis, coupled with advanced Marfey's analysis to determine the absolute stereochemistry of the amino acids.

The sponge Inflatella coelosphaeroides, obtained from a deep-water trawl on the Scotia Arc of the Southern Ocean as previously described²¹, was freeze-dried, extracted with 1:1 dichloromethane/methanol, and the extract fractionated by medium pressure liquid chromatography.

The mid-polarity fractions contained metabolites bearing multiple N-methyl groups, reminiscent of friomaramide (friomaramide A) (P1) and were subsequently purified by HPLC to produce seven new highly methylated peptides, including friomaramide B (P2) and six peptides without the characteristic tryptenamine function found on friomaramides, herein named shagamides A-F (3-8).

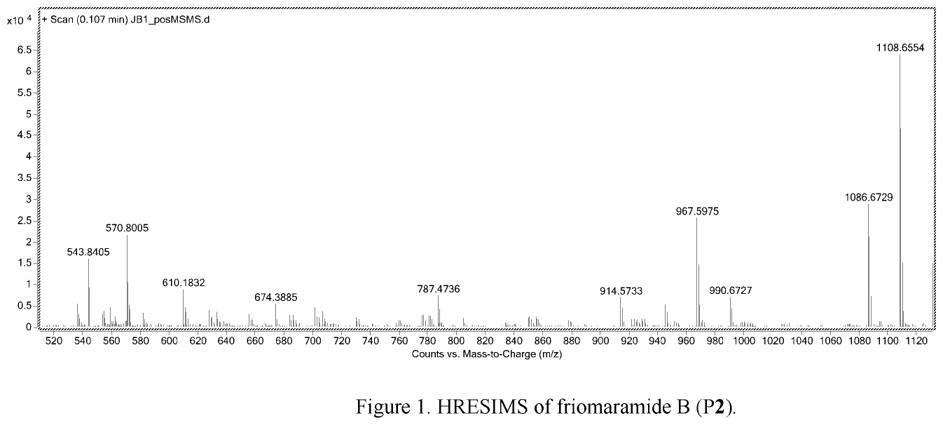

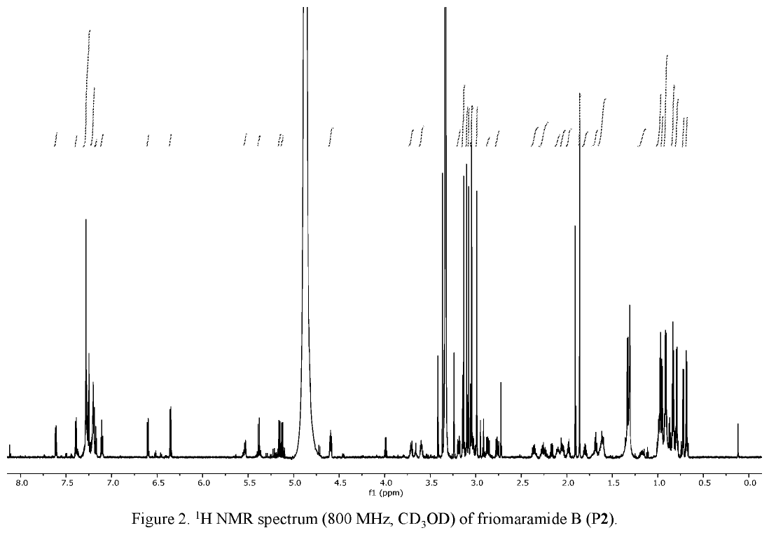

Friomaramide B (P2) was isolated as a white film, with a molecular formula C₆₂H₈₇N₉O₈ (24 double bond equivalents) established by analysis of the protonated molecule at m/z 1086.6729 in the (+)-HRESIMS (FIG. 1). Interpretation of the 1D ¹H NMR spectrum (FIG. 2) in conjunction with 2D COSY, HSQC, HMBC and HSQC-TOCSY NMR data (Table 1) suggested the presence of the proteinogenic amino acids phenylalanine (×2), proline, leucine, valine (×2) and isoleucine. Similar to friomaramide A (P1), the data also gave evidence for five N-methyl groups, an acetyl group and a decarboxylated tryptophan residue (i.e., trypteneamine). The carbonyl and amino acid side chain groups accounted for all 24 degrees of unsaturation, therefore P2 must be linear.

TABLE 1
NMR data for friomaramide B (P2) (CD3OD).

| pos | δ_{C}, type^{a} | δ_{H}^{b} | gCOSY | gHMBC |
|---|---|---|---|---|
| Acetyl | | | | |
| CO | 172.9, C | | | |
| Me | 22.4, CH₃ | 1.84, s | | CO |
| Phe¹ | | | | |
| CO | 173.2, C | | | |
| α | 55.9, CH | 4.58, dd (9.8, 5.3) | β | β, γ, CO, acetyl-CO |
| β | 38.7, CH₂ | 3.02, o/l^{c} | α | α, δ, γ |
| | | 2.74 dd (13.9, 9.2) | α | α, δ, γ, CO |
| γ | 138.5, C | | | |
| δ | 130.2, CH | 7.18, o/l | ε | β, ζ |
| ε | 129.3, CH | 7.24, o/l | δ, ζ | γ |
| ζ | 127.6, CH | 7.19, o/l | ε | δ |
| Phe² | | | | |
| CO | 171.3, C | | | |
| α | 53.8, CH | 4.84, o/l | β | β, γ, CO, Phe¹-CO |
| β | 38.1, CH₂ | 3.17, dd (14.9, 4.9) | α | α, δ, γ, CO |
| | | 2.85, dd (14.3, 8.8) | α | α, δ, γ, CO |
| γ | 138.4, C | | | |
| δ | 130.6, CH | 7.26, o/l | ε | β, ζ |
| ε | 129.3, CH | 7.26, o/l | δ, ζ | γ |
| ζ | 127.6, CH | 7.19, o/l | ε | δ |
| Pro³ | | | | |
| CO | 174.1, C | | | |
| α | 58.8, CH | 4.81, o/l | β | β |
| β | 29.7, CH₂ | 2.25, m^{d} | α, γ | δ, γ, CO |
| | | 1.78, m | α, γ | δ, γ, CO |
| γ | 25.9, CH₂ | 2.02, m | β, δ | α, β, δ |
| | | 1.96, m | β, δ | α, β, δ |
| δ | 48.6, CH₂ | 3.69, m | γ | β, γ |
| | | 3.57, m | γ | β, γ |
| N-MeLeu⁴ | | | | |
| CO | 173.8, C | | | |
| α | 53.0, CH | 5.52, dd (8.4, 6.0) | β | β, N-Me, γ, CO |
| β | 38.9, CH₂ | 1.66, m | α, γ | α, γ, δ, δ′, CO |
| | | 1.59, o/l | α, γ | α, γ, δ, δ′ |
| γ | 26.1, CH | 1.58, o/l | β, δ, δ′ | |
| δ | 22.8, CH₃ | 0.93, d (6.1) | γ | δ′, γ, β |
| δ′ | 23.2, CH₃ | 0.95, d (6.2) | γ | δ, γ, β |
| N-Me | 31.3, CH₃ | 3.11, s | | α, Pro3-CO |
| N-MeVal⁵ | | | | |
| CO | 172.1, C | | | |
| α | 60.0, CH | 5.14, d (10.8) | β | β, γ, γ′, N-Me, CO, N-MeLeu⁴-CO |
| β | 28.4, CH | 2.34, m | α, γ, γ′ | α, γ, γ′ |
| γ | 18.7, CH₃ | 0.81, o/l | β | α, β, γ′ |
| γ′ | 19.8, CH₃ | 0.89, o/l | β | α, β, γ |
| N-Me | 30.9, CH₃ | 3.03, s | | α, N-MeLeu4-CO |
| N-MeVal⁶ | | | | |
| CO | 171.3, C | | | |
| α | 60.0, CH | 5.11, d (10.7) | β | β, γ, γ′, N-Me, CO, N-MeVal⁵-CO |
| β | 28.4, CH | 2.22, m | α, γ, γ′ | |
| γ | 19.6, CH | 0.66, d (6.5) | β | α, β, γ′ |
| γ′ | 18.4, CH | 0.70, d (6.7) | β | α, β, γ |
| N-Me | 31.1, CH₃ | 2.97, s | | α, N-MeVal⁵-CO |
| N-MeIle⁷ | | | | |
| CO | 172.3, C | | | |
| α | 59.5, CH | 5.36, d (10.9) | β | β, γ, β-Me, N-Me, CO, N-MeVal⁶-CO |
| β | 34.6, CH | 2.08, m | α, β-Me | |
| β-Me | 16.1, CH₃ | 0.77, d (6.5) | β | α, β, γ |
| γ | 25.2, CH₂ | 0.90, o/l | δ | |
| | | 1.16, m | δ | |
| δ | 11.3, CH₃ | 0.81, o/l | γ | β, δ |
| N-Me | 31.5, CH₃ | 3.06, s | | α, N-MeVal⁶-CO |
| N-MeTrp-ene | | | | |
| 1 | 124.6, CH | 6.34, d (8.2) | 1 | 2, 3′ |
| 2 | 120.9, CH | 6.58, d (8.3) | 2 | 1, 2′, 3a′ |
| 2′ | 126.0, CH | 7.23, s | | 3′, 3a′, 7a′ |
| 3′ | 110.6, C | | | |
| 3a′ | 128.2, C | | | |
| 4′ | 119.5, CH | 7.59, dt (8.0, 1.0) | 5′ | 3′, 6′, 7a′ |
| 5′ | 121.0, CH | 7.09, ddd (8.0, 7.0, 1.0) | 4′, 6′ | 3a′, 7′ |
| 6′ | 123.3, CH | 7.16, ddd (8.1, 7.0, 1.1) | 5′, 7′ | 4′, 7a′ |
| 7′ | 112.6, CH | 7.37, dt (8.1, 0.9) | 6′ | 3a′, 5′ |
| 7a′ | 137.3, C | | | |
| N-Me | 34.4. CH₃ | 3.08, s | | 1, N-Melle⁷-CO |

^{a}Shift (200 MHz), type, determined by HSQC;
^{b}Shift (800 MHz), multiplicity, J (Hz);
^{c}overlapping signals;
^{d}multiplet

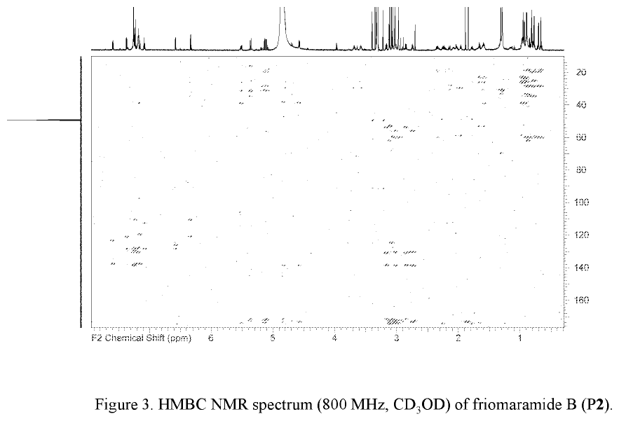

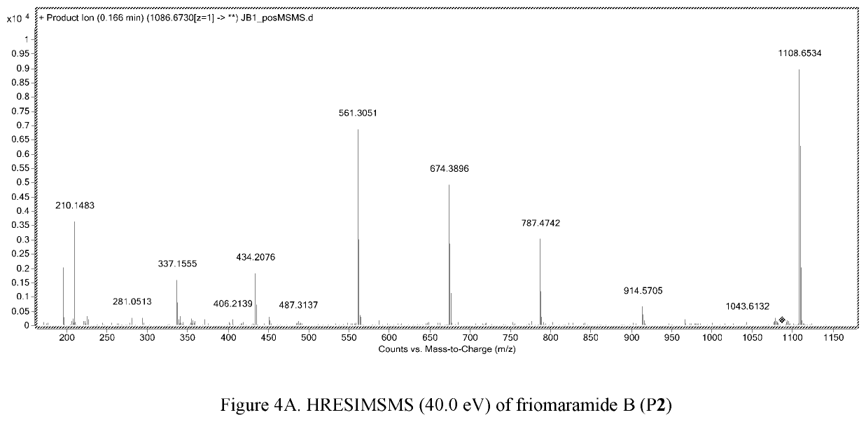

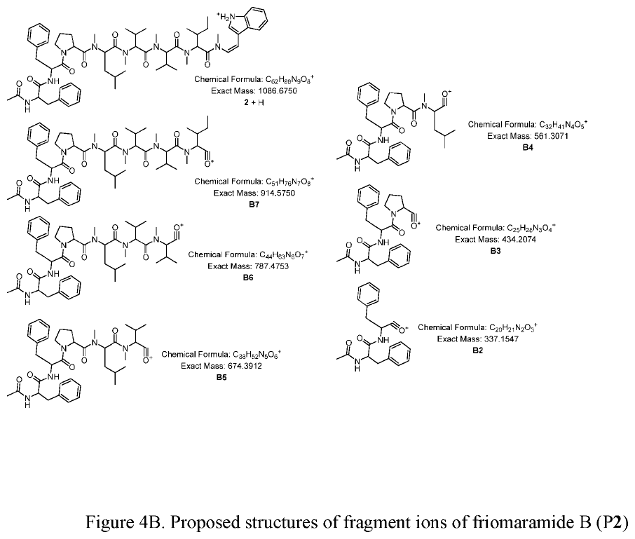

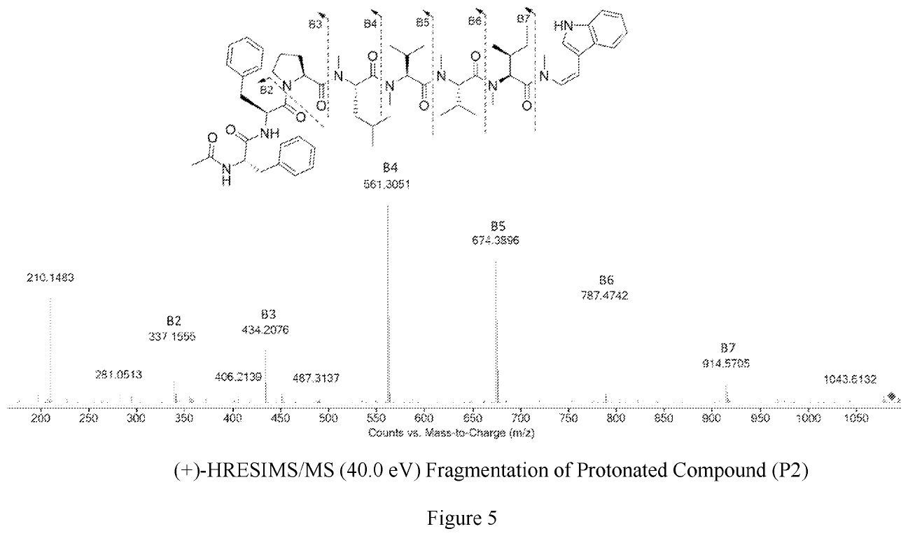

The sequence of amino acids was determined by a combination of HMBC correlations (FIG. 3) between neighboring residues and MSMS fragmentation analysis (FIG. 4A-B). As with friomaramide A (P1), the trypteneamine residue must be placed at the C-terminus due to the lack of carboxylic acid functionality. Correlations in the HMBC spectrum from the N-methyl at δ_{H} 5.49 to the olefinic C-1 (δ_{C} 124.6) and N-MeIle⁷-CO (δ_{C} 172.3) indicated the tryptenamine nitrogen is methyl bearing and N-Melle⁷ is the next residue. These are both confirmed by the two fragment ions in the MSMS spectrum at m/z 914.5705 and 787.4742 corresponding to the B7 and B6 ions respectively (FIG. 5).

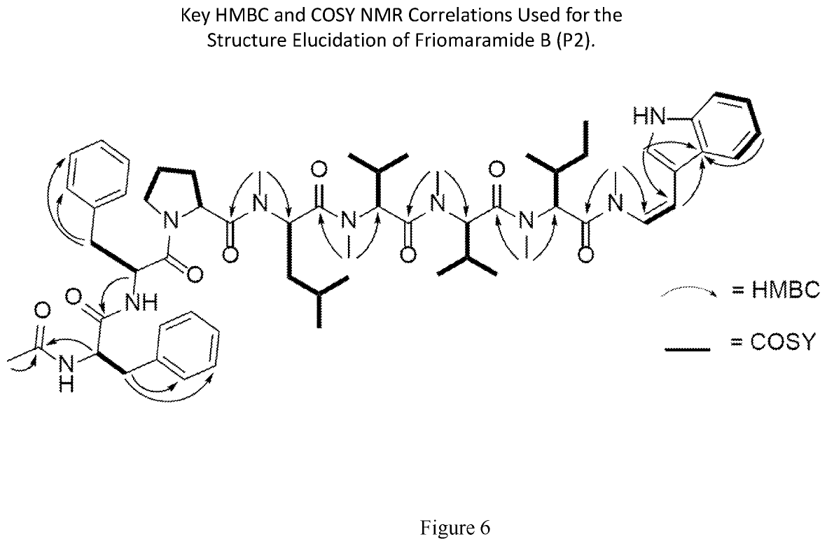

The process of connecting the adjacent amino acid by N-methyl HMBC correlations to the neighboring CO and with fragmentation data was repeated, connecting N-MeIle⁷ to N-MeVal⁶ followed by N-MeVal⁵, N-MeLeu⁴ and finally to Pro³. The N-terminus was established as acetyl-Phe¹ based on an HMBC correlation from Phe¹-α (δ_{H} 4.58) to the acetyl carbonyl (δ_{C} 172.3). The planar structure was ultimately completed by observing HMBC correlations from Phe²-α (δ_{H} 4.84) to Phe¹-CO (δ_{C} 173.2) and the B3 MSMS fragment m/z 434.2076 derived from acetyl-Phe¹-Phe²-Pro³ (FIG. 6).

The olefin of the trypteneamine residue was assigned a cis-orientation, as the ³J_{H,H}=8.2 Hz coupling constant is consistent with other previously reported metabolites,²²⁻²³ whereas those containing trans-orientations are typically much larger.²⁴ Stereochemical analysis using the advanced Marfey's method was then used to establish the absolute configuration of the amino acid residues in P2.²⁵ A small sample of the peptide was first hydrolyzed under acidic conditions, and the resulting amino acids were derivatized with Marfey's reagent FDAA (N_{α}-(2,4-dinitro-5-fluorophenyl-L-alaninamide). LC-HRESIMS was then used to compare the retention times of the reaction products to both D and L synthesized amino acid standards using methods previously established.²⁶ All of the canonical and N-methylated D and L amino acid isomers could be resolved using a C18 column and a linear gradient of 25% acetonitrile (ACN) (0.1% formic acid) to 100% over 40 min. By comparison of the hydrolyzed sample to the standards, this method proved all the amino acids where of the natural proteinogenic L configuration (Table 2) thus completing the 3D structure of friomaramide B (P2).

TABLE 2
Advanced Marfey's analysis of friomaramide B (P2).

| | | Retention Time (min) | | | Absolute |
|---|---|---|---|---|---|
| Amino | | D stan- | L Stan- | Friomaramide B | Configura- |
| Acids | m/z | dard | dard | (2) Hydrosylate | tion |
| Phe | 418.136 | 12.1 | 10.6 | 10.5 | L |
| Pro | 368.120 | 6.6 | 6.1 | 6.0 | L |
| N-MeLeu | 398.167 | 12.2 | 11.5 | 11.4 | L |
| N-MeVal | 384.151 | 11.2 | 10.1 | 10.1 | L |
| N-MeIle | 398.167 | 13.0 | 11.8 | 11.6 | L |
| N-MeAlloIle | 398.167 | 13.2 | 11.9 | — | — |

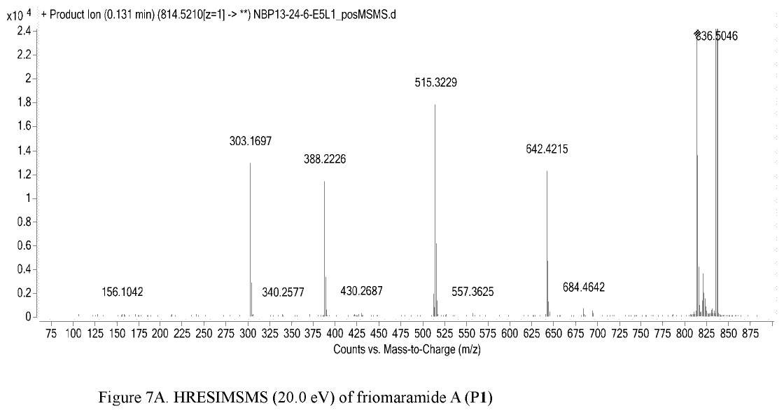

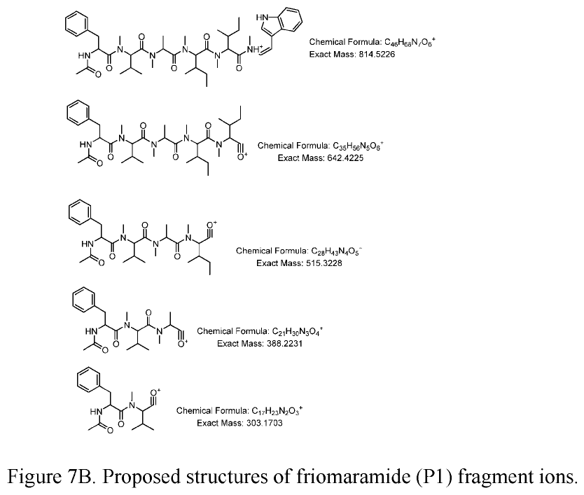

Aside from the nature of amino acid residues, several differences between the originally proposed structure of friomaramide A (P1) and that deduced for friomaramide B prompted re-isolation and analysis of its NMR and MS data (Table 15). In doing so, HMBC correlations from the signal at δ_{H} 3.08 to N-MeIle⁵-CO (δ_{C} 172.2) and olefinic C-1 (δ_{C} 124.5) revealed the trypteneamine to bear an N-methyl group, while no methyl groups were observed to correlate with Phe¹. This was backed by the observed fragment ions in the MSMS spectrum (FIG. 7A-B). Friomaramide A was then subjected to a stereochemical analysis using the advanced Marfey's method, where Phe¹ was determined to have the L configuration (Table 16). not D as originally proposed, while the rest of the amino acids also showed the L configuration. Thus, the revised structure of friomaramide A is consistent with that determined for friomaramide B.

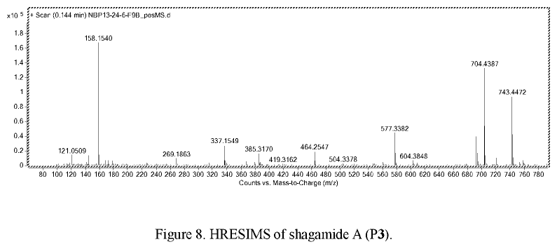

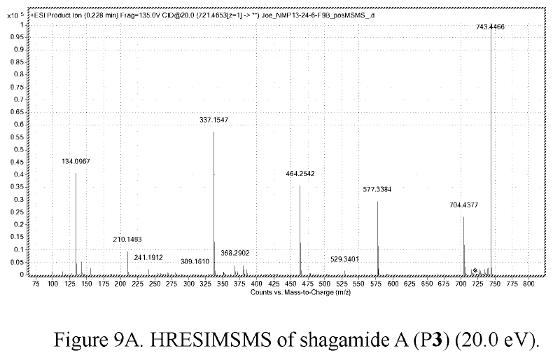

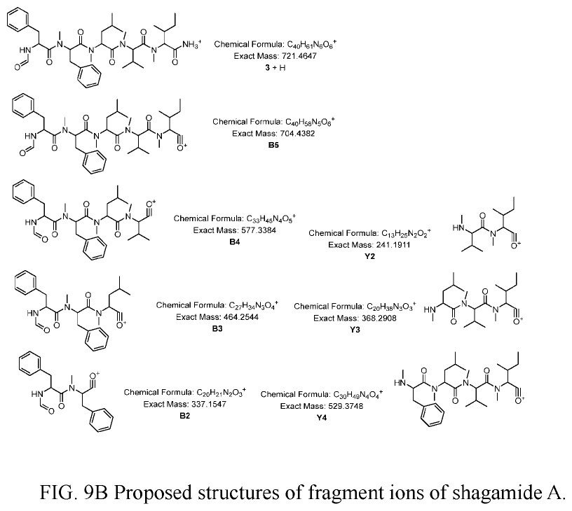

Shagamide A (P3) was isolated as a white solid, with a molecular formula of C₄₀H₆₀N₆O₆ inferred from the sodium adduct at m/z 743.4472 detected in the HRESIMS (FIG. 8). The 1D ¹H NMR spectrum (FIG. 9) showed signals representative of a penta-peptide with four nitrogen-bearing methyl groups, and the presence of a formyl group (δ_{H} 7.98, s). Correlations in the 2D NMR spectra suggested 3 was made up of an N-Melle, N-MeVal, N-MeLeu, N-MePhe and a Phe residue, with the HMBC from the formyl signal to α-Phe (δ_{C} 50.4) suggesting the only residue lacking N-methylation is formylated (Table 3). As above, HMBC correlations from the N-methyl ¹H NMR signal to the adjacent CO carbon was used alongside MSMS fragmentation data to establish the sequence of amino acids as Phe-N-MePhe-N-MeLeu-N-MeVal-N-MeIle. Finally, the [M-NH₂]⁺ B5 MSMS peak at m/z 704.4382 (FIG. 9A-B) provided strong evidence for a free primary amide at the C-terminus to complete the planar structure of P3, while the advanced Marfey's analysis (Table 4) confirmed all amino acids were L configured.

TABLE 3
NMR data for shagamide A (P3) (CD₃OD)

| pos | δ_{C},^{a} type | δ_{H}^{b} | gCOSY | gHMBC |
|---|---|---|---|---|
| Formyl | | | | |
| COH | 162.9, CH | 7.98, s | α-Phe¹ | α-Phe¹ |
| Phe¹ | | | | |
| CO | 172.7, C | | | |
| α | 50.4, CH | 5.23, br dd (9.0, 6.5) | COH, β | |
| β | 39.1, CH₂ | 2.95, m^{c} | α | α, γ, δ |
| | | 3.06, m | α | |
| γ | 137.6, C | | | |
| δ | 130.4, CH | 7.28, o/l^{d} | | |
| ε | 129.7, CH | 7.28, o/l | | |
| ζ | 128.2, CH | 7.25, o/l | | |
| N-MePhe² | | | | |
| CO | 170.3, C | | | |
| α | 57.0, CH | 5.55, br dd (9.4, 5.5) | β | |
| β | 36.0, CH₂ | 2.71, dd (13.3, 5.6) | α | α, γ, δ |
| | | 3.22, dd (13.2, 9.4) | α | |
| γ | 138.5, C | | | |
| δ | 130.6, CH | 7.28, o/l | | |
| ε | 129.4, CH | 7.22, o/l | | |
| ζ | 127.7, CH | 7.18, o/l | | |
| N-Me | 31.5, CH₃ | 2.99, s | | α, Phe¹-CO |
| N-MeLeu³ | | | | |
| CO | 172.3, C | | | |
| α | 52.7, CH | 5.30, t (7.1) | β | N-Me, β, CO |
| β | 39.2, CH₂ | 1.22, m | α, γ | |
| | | 1.53, m | α, γ | |
| γ | 25.9, CH₂ | 1.23, m | β, δ, δ′ | |
| δ | 22.7, CH₃ | 0.91, o/l | γ | δ′, γ, β |
| δ′ | 23.0, CH₃ | 0.90, o/l | γ | δ, γ, β |
| N-Me | 30.7, CH₃ | 2.40, s | | α, N-MePhe²-CO |
| N-MeVal⁴ | | | | |
| CO | 172.5, C | | | |
| α | 59.8, CH | 5.00, d (10.9) | β | N-Me, β, γ′, CO |
| β | 28.5, CH | 2.12, m | α, γ, γ′ | |
| γ | 18.8, CH₃ | 0.56, d (6.7) | β | α, β, γ′ |
| γ′ | 19.5, CH₃ | 0.85, o/l | β | α, β, γ |
| N-Me | 30.5, CH₃ | 2.49, s | | α, N-MeLeu³-CO |
| N-MeIle⁵ | | | | |
| CO | 174.2, C | | | |
| α | 61.2, CH | 4.69, d (11.3) | β | N-Me, β, N-MeVal⁴-CO, CO |
| β | 33.0, CH | 2.02, m | α, γ, β-Me | |
| β-Me | 15.6, CH₃ | 0.92, o/l | β | α, β, γ |
| γ | 25.4, CH₂ | 0.90, m | β, δ | |
| | | 1.20, m | | |
| δ | 10.6, CH₃ | 0.84, o/l | γ | γ, β |
| N-Me | 31.4, CH₃ | 3.01, s | | α, N-MeVal⁴-CO |

^{a}Shift (125 MHz), type, determined by HSQC;
^{b}Shift (500 MHz), multiplicity, J (Hz);
^{c}multiplet;
^{d}overlapping signals

TABLE 4
Advanced Marfey's analysis of compound shagamide A (P3)

| | | Retention Time (min) | | | Absolute |
|---|---|---|---|---|---|
| Amino | | D | L | Shagamide A | Configura- |
| Acids | m/z | standard | Standard | (3) Hydrosylate | tion |
| Phe | 418.136 | 12.1 | 10.6 | 10.6 | L |
| N-MePhe | 432.151 | 10.9 | 10.7 | 10.7 | L |
| N-MeLeu | 398.167 | 12.2 | 11.5 | 11.5 | L |
| N-MeVal | 384.151 | 11.2 | 10.1 | 10.0 | L |
| N-MeIle | 398.167 | 13.0 | 11.8 | 11.7 | L |
| N-MeAlloIle | 398.167 | 13.2 | 11.9 | — | — |

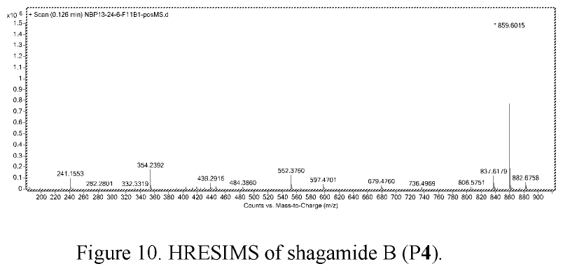

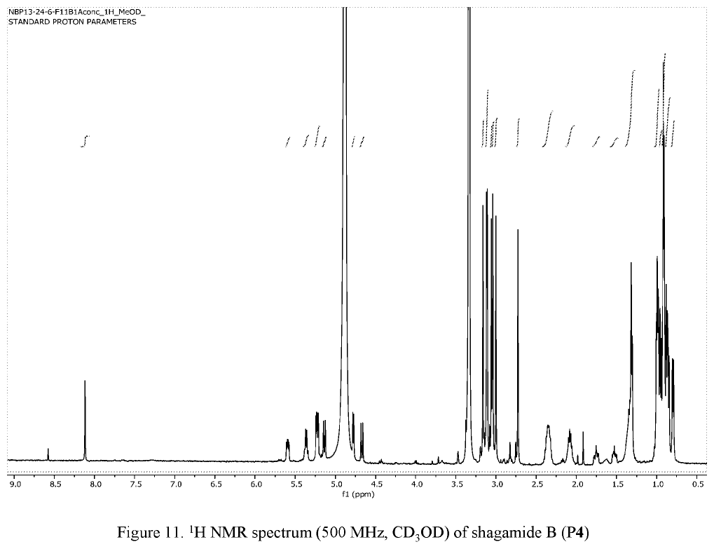

Shagamide B (P4) was isolated as a white solid, with a molecular formula of C₄₃H₈₀N₈O₈ inferred from the sodium adduct at m/z 859.6015 detected in the HRESIMS (FIG. 10). Similar to P3, the ¹H NMR spectrum (FIG. 11) showed evidence of a heavily N-methylated peptide with a formyl group (δ_{H} 8.09, s), with 2D NMR spectra providing evidence for Val, N-MeVal (×3), N-MeIle (×2) and N-MeAla residues (Table 5). The order of the residues was established as Val-N-MeVal-N-MeVal-N-MeAla-N-MeVal-N-MeLeu-N-MeIle. The C-terminus was shown to be an N-methylamide, both by the HMBC correlation from the signal at δ_{H} 2.70 to N-MeIle⁷-CO (δ_{C} 172.5) only, and also the [M-NHCH₃]⁺ B7 MSMS peak at m/z 806.5750 (FIG. 10), while the N-terminal Val was N-formylated deduced by mutual HMBC correlations from the ¹H NMR signals of the formyl group and α-Val¹ (δ_{H} 4.75) to each other's ¹³C NMR resonance (δ_{C} 163.4 and 54.7 respectively). Using the LC-HRESIMS method, all the amino acids were determined to be the L isomer (Table 6). The D and L N-MeAla standards were inseparable using that method but resolution of the two isomers could be achieved using the same C18 column with a gradient using H₂O and MeOH (both with 5% ACN and 0.1% formic acid modifier, see Experimental),²⁶ facilitating comparison to the hydrolyzed shagamide B sample and confirming it as the L isomer.

TABLE 5
NMR data for compound shagamide B (P4) (CD₃OD)

| pos | δ_{C}, type^{a} | δ_{H}^{b} | gCOSY | gHMBC |
|---|---|---|---|---|
| Formyl | | | | |
| COH | 163.4, CH | 8.09, s | α-Val¹ | α-Val¹ |
| Val¹ | | | | |
| CO | 174.0, C | | | |
| α | 54.7, CH | 4.75, d (7.3) | β | γ, β, COH, CO |
| β | 31.4, CH | 2.06, m^{c} | α, γ, γ′ | α, γ, γ′ |
| γ | 18.0, CH₃ | 0.96, o/l^{d} | β | γ′, β, α |
| γ′ | 19.8, CH₃ | 0.96, o/l | β | γ, β, α |
| N-MeVal² | | | | |
| CO | 171.9, C | | | |
| α | 59.6, CH | 5.21, d (10.7) | β | N-Me, β, γ′, γ, CO |
| β | 28.5, CH | 2.33, m | α, γ, γ′ | α, γ′ |
| γ | 18.4, CH₃ | 0.82, d (6.9) | β | α, β, γ′ |
| γ′ | 19.6, CH₃ | 0.89, o/l | β | β, γ |
| N-Me | 31.0, CH₃ | 3.14, s | | α, Val¹-CO |
| N-MeVal³ | | | | |
| CO | 171.6, C | | | |
| α | 59.7, CH | 5.19, d (10.7) | β | N-Me, β, γ′, γ, CO |
| β | 28.3, CH | 2.33, m | α, γ, γ′ | α, γ′ |
| γ | 18.4, CH₃ | 0.78, d (6.8) | β | α, β, γ′ |
| γ′ | 19.8, CH₃ | 0.89, o/l | β | β, γ |
| N-Me | 31.0, CH₃ | 3.02, s | | α, N-MeVal²-CO |
| N-MeAla⁴ | | | | |
| CO | 174.6, C | | | |
| α | 51.5, CH | 5.34, q (6.9) | β | β, N-Me, CO |
| β | 14.8, CH₃ | 1.28, d (6.9) | α | α, CO |
| N-Me | 31.5, CH₃ | 3.10, s | | α, N-MeVal³-CO |
| N-MeVal⁵ | | | | |
| CO | 171.8, C | | | |
| α | 60.0, CH | 5.11, d (10.8) | β | N-Me, β, γ′, γ, CO |
| β | 28.3, CH | 2.33, m | α, γ, γ′ | |
| γ | 18.7, CH₃ | 0.84, d (7.4) | β | α, β, γ′ |
| γ′ | 19.7, CH₃ | 0.89, o/l | β | β, γ |
| N-Me | 30.5, CH₃ | 3.03, s | | α, N-MeAla⁴-CO |
| N-MeLeu⁶ | | | | |
| CO | 173.3, C | | | |
| α | 52.7, CH | 5.57, dd (9.9, | β | N-Me, CO |
| | | 5.2) | | |
| β | 38.5, CH₂ | 1.74, m | α, γ | |
| | | 1.50, m | α, γ | γ |
| γ | 25.8, CH | 1.34, o/l | β, δ, δ′ | |
| δ | 22.1, CH₃ | 0.92, o/l | γ | δ′, γ, β |
| δ′ | 23.4, CH₃ | 0.94, o/l | γ | δ, γ, β |
| N-Me | 31.1, CH₃ | 2.98, s | | α, N-MeVal⁵-CO |
| N-MeIle⁷ | | | | |
| CO | 172.5, C | | | |
| α | 61.9, CH | 4.64, d (11.2) | β | N-Me, β, CO |
| β | 33.4, CH | 2.07, m | α, β-Me | |
| β-Me | 15.6, CH₃ | 0.89, o/l | β | α, β, γ |
| γ | 25.6, CH₂ | 1.31, o/l | δ | |
| δ | 10.5, CH₃ | 0.85, o/l | γ | β, γ |
| N-Me | 31.3, CH₃ | 3.09, s | | α, N-MeLeu⁶-CO |
| N-Me | 26.0, CH₃ | 2.70, s | | N-MeIle⁷-CO |

^{a}Shift (125 MHz), type, determined by HSQC;
^{b}Shift (500 MHz), multiplicity, J (Hz);
^{c}multiplet;
^{d}overlapping signals

TABLE 6
Advanced Marfey's analysis of compound shagamide B (P4)

| | | Retention Time (min) | | | |
|---|---|---|---|---|---|
| | | | | Shagamide B | |
| Amino | | | | (P4) | Absolute |
| Acids | m/z | D standard | L Standard | Hydrosylate | Configuration |
| Val | 370.135 | 10.3 | 8.4 | 8.3 | L |
| N-MeAla | 356.120 | 37.7 | 38.1 | 38.1 | L |
| N-MeVal | 384.151 | 11.2 | 10.1 | 9.9 | L |
| N-MeIle | 398.167 | 12.8 | 11.5 | 11.3 | L |
| N-MeAlloIle | 398.167 | 13.2 | 11.9 | — | — |

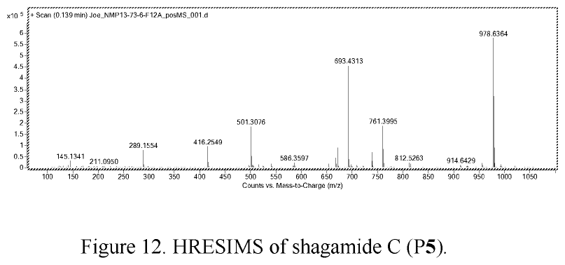

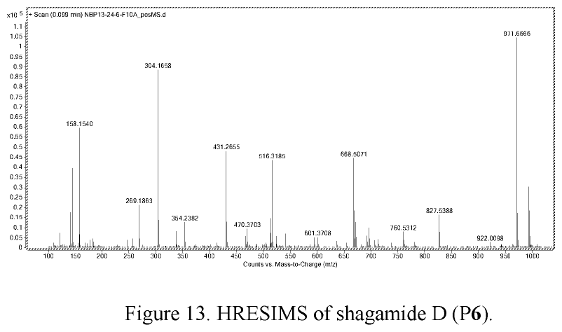

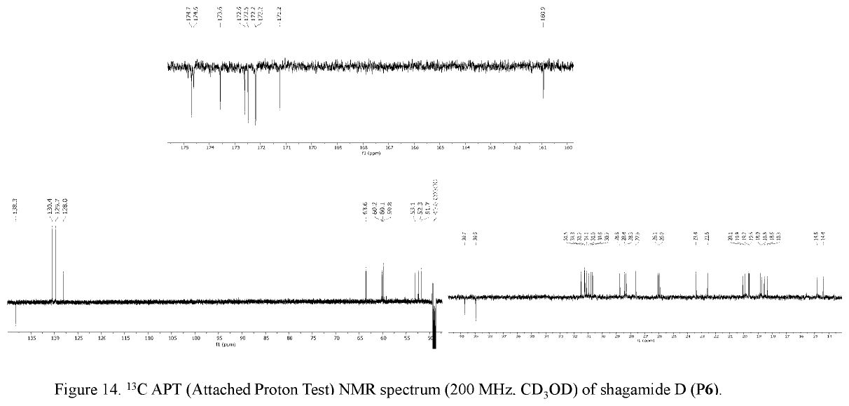

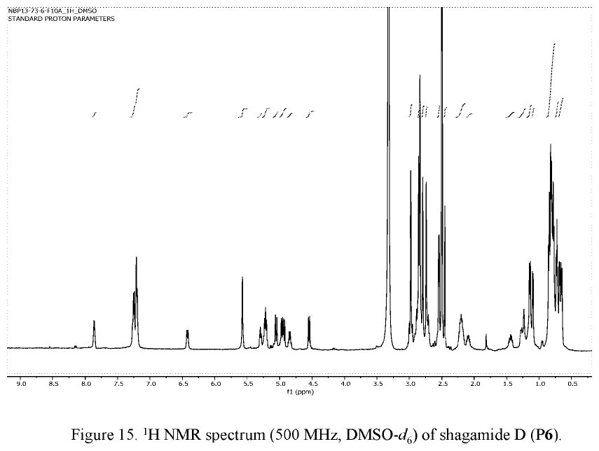

Shagamide C (P5) and shagamide D (P6) were both isolated as a white solids and shared significant spectroscopic data. Shagamide C was found with a molecular formula of C₅₀H₈₅N₉O₉ inferred from the sodium adduct at m/z 978.6364 detected in the HRESIMS (FIG. 12). By the same methods as above, P5 was found to include a Phe-N-MeVal-N-MeLeu-N-MeAla-N-MeAla-N-MeVal-N-MeVal-N-MeVal linear octapeptide, with the Phe residue formylated at the N-terminus and the C-terminus also an N-methylamide (Table 7). Shagamide D was found with 1D and 2D NMR data (Table 8) suggesting the same linear octapeptide backbone, however the molecular formula of C₅₀H₈₆N₁₀O₉, inferred from the protonated molecule detected at m/z 971.6666 in the HRESIMS (FIG. 13), has an extra NH and the ¹H NMR spectrum lacked a signal for the formyl group. Instead, the N-terminus is a mono-substituted ureido moiety, inferred from both the ¹³C APT spectrum (FIG. 14) and the NH₂ resonance (8 5.57, 500 MHz) that showed a ROESY correlation to Phe-NH (δ_{H} 6.43) in the ¹H NMR spectrum acquired in DMSO-d₆ (FIG. 15). The amino acids were determined to be all L configured for both compounds P5 and P6 (Table 9 and Table 10, respectively).

TABLE 7
NMR data for compound shagamide C (P5) (CD₃OD).

| pos | δ_{C}, type^{a} | δ_{H}^{b} | gCOSY | gHMBC |
|---|---|---|---|---|
| Formyl | | | | |
| COH | 162.9, CH | 8.01, s | α-Phe¹ | α-Phe¹ |
| Phe¹ | | | | |
| CO | 173.1, C | | | |
| α | 50.5, CH | 5.27, o/l^{c} | β | β, CO |
| β | 39.0, CH₂ | 3.08, m^{d} | α | α, γ, δ |
| | | 2.90, m | | α, γ, δ, CO |
| γ | 137.7, C | | | |
| δ | 130.3, CH | 7.28, m | ε | ε |
| ε | 129.6, CH | 7.29, m | δ, ζ | γ |
| ζ | 128.1, CH | 7.24, m | ε | δ |
| N-MeVal² | | | | |
| CO | 171.1, C | | | |
| α | 60.1, CH | 5.04, d (10.7) | β | N—Me, β, γ, CO, Phe¹-CO |
| β | 28.4, CH | 2.30, m | α, γ, γ′ | α |
| γ | 18.3, CH₃ | 0.81, o/l | β | α, β, γ′ |
| γ′ | 19.9, CH₃ | 0.87, o/l | β | α, β, γ |
| N—Me | 30.8, CH₃ | 2.97, s | | α, Phe¹-CO |
| N-MeLeu³ | | | | |
| CO | 172.1, C | | | |
| α | 53.1, CH | 5.44, t (7.4) | β | N—Me, β, CO |
| β | 38.9, CH₂ | 1.57, m | α, γ | α, γ, δ, δ′ |
| | | 1.33, o/l | α, γ | α, γ, δ, δ′ |
| γ | 25.9, CH | 1.27, m | β, δ, δ′ | |
| δ | 23.2, CH₃ | 0.92, o/l | γ | β, γ, δ′ |
| δ′ | 22.5, CH₃ | 0.93, o/l | γ | β, γ, δ |
| N—Me | 30.9, CH₃ | 2.62, s | | α, N-MeVal²-CO |
| N-MeAla⁴ | | | | |
| CO | 173.5, C | | | |
| α | 52.2, CH | 5.28, o/l | β | β, N—Me, CO |
| β | 14.3, CH₃ | 1.25, d (6.9) | α | α, CO |
| N—Me | 31.1, CH₃ | 2.93, s | | α, N-MeLeu³-CO |
| N-MeAla⁵ | | | | |
| CO | 174.6, C | | | |
| α | 51.6, CH | 5.31, o/l | β | β, CO |
| β | 14.7, CH₃ | 1.30, d (6.9) | α | α, CO |
| N—Me | 31.1, CH₃ | 3.00, s | | α, N-MeAla⁴-CO |
| N-MeVal⁶ | | | | |
| CO | 172.1, C | | | |
| α | 60.0, CH | 5.11, d (10.8) | β | N—Me, β, γ, γ′, CO, N-MeVal⁵-CO |
| β | 28.2, CH₂ | 2.32, o/l | α, γ, γ′ | α, γ, γ′ |
| γ | 18.7, CH₃ | 0.84, o/l | β | α, β, γ′ |
| γ′ | 19.8, CH₃ | 0.88, o/l | β | α, β, γ |
| N—Me | 30.6, CH₃ | 3.02, s | | α, N-MeAla⁵-CO |
| N-MeVal⁷ | | | | |
| CO | 172.5, C | | | |
| α | 59.7, CH | 5.18, d (10.7) | β | N—Me, β, γ, γ′, CO, N-MeVal⁶-CO |
| β | 28.7, CH₂ | 2.33, o/l | α, γ, γ′ | α |
| γ | 18.5, CH₃ | 0.78, o/l | β | α, β, γ′ |
| γ′ | 19.6, CH₃ | 0.90, o/l | β | α, β, γ |
| N—Me | 31.0, CH₃ | 3.01, s | | α, N-MeVal⁶-CO |
| N-MeVal⁸ | | | | |
| CO | 172.4, C | | | |
| α | 63.5, CH | 4.61, d, (11.0) | β | N—Me, β, γ, γ′ |
| β | 27.5, CH₂ | 2.24. m | α, γ, γ′ | α, γ, γ′ |
| γ | 18.7, CH₃ | 0.76, d (6.7) | β | α, β, γ′ |
| γ′ | 19.5, CH₃ | 0.92, o/l | β | α, β, γ |
| N—Me | 31.4, CH₃ | 3.11, s | | α, N-MeVal⁷-CO |
| N—Me | 26.0, CH₃ | 2.70, s | | N-MeVal⁸-CO |

^{a}Shift (125 MHz), type, determined by HSQC;
^{b}Shift (500 MHz), multiplicity, J (Hz);
^{c}overlapping signals;
^{d}multiplet

TABLE 8
NMR data for shagamide D (P6) (CD₃OD).

| pos | δ_{C}, type^{a} | δ_{H}^{b} | gCOSY | gHMBC |
|---|---|---|---|---|
| Amido | | | | |
| CO | 160.9 | | | |
| Phe¹ | | | | |
| CO | 174.6, C | | | |
| α | 52.3, CH | 5.05, o/l^{c} | β | β, CO |
| β | 39.7, CH₂ | 2.99, o/l | α | α, γ, δ, CO |
| | | 2.85, dd (13.7, 7.0) | | α, γ, δ, CO |
| γ | 138.3, C | | | |
| δ | 130.4, CH | 7.26, o/l | ε | ε |
| ε | 129.7, CH | 7.27, o/l | δ, ζ | γ |
| ζ | 128.0, CH | 7.23, o/l | ε | δ |
| N-MeVal² | | | | |
| CO | 171.2, C | | | |
| α | 60.2, CH | 5.03, d (10.4) | β | β, γ, γ′, N—Me, CO |
| β | 28.3, CH | 2.28, m^{d} | α, γ, γ′ | α, γ, γ′ |
| γ | 18.3, CH₃ | 0.81, o/l | β | α, β, γ′ |
| γ′ | 20.1, CH₃ | 0.86, o/l | β | α, β, γ |
| N—Me | 30.8, CH₃ | 2.95, s | | α, Phe¹-CO |
| N-MeLeu³ | | | | |
| CO | 172.2, C | | | |
| α | 53.1, CH | 5.43, t (7.4) | β | β, N—Me, CO |
| β | 39.0, CH₂ | 1.55, dt (14.2, 7.3) | α, γ | α, γ, δ, δ′ |
| | | 1.33, o/l | α, γ | α, γ, δ, δ′ |
| γ | 26.0, CH | 1.24, o/l | β, δ, δ′ | |
| δ | 23.4, CH₃ | 0.91, o/l | γ | δ′, γ, β |
| δ′ | 22.6, CH₃ | 0.92, o/l | γ | δ, γ, β |
| N—Me | 31.0, CH₃ | 2.57, s | | α, N-MeVal²-CO |
| N-MeAla⁴ | | | | |
| CO | 173.6, C | | | |
| α | 52.3, CH | 5.27, o/l | β | β, N—Me, CO |
| β | 14.4, CH₃ | 1.24, d (6.9) | α | α, CO |
| N—Me | 31.3, CH₃ | 2.92, s | | α, N-MeLeu³-CO |
| N-MeAla⁵ | | | | |
| CO | 174.7, C | | | |
| α | 51.7, CH | 5.30, o/l | β | β, N—Me, CO |
| β | 14.8, CH₃ | 1.30, d (7.1) | α | α, CO |
| N—Me | 31.2, CH₃ | 3.00, s | | α, N-MeAla⁴-CO |
| N-MeVal⁶ | | | | |
| CO | 172.2, C | | | |
| α | 60.1, CH₃ | 5.11, d (10.8) | β | N—Me, β, γ, γ′, CO, N-MeAla⁵-CO |
| β | 28.4, CH | 2.32, m | α, γ, γ′ | α, γ, γ′ |
| γ | 18.8, CH₃ | 0.84, o/l | β | α, β, γ′ |
| γ′ | 19.9, CH₃ | 0.87, 0/l | β | α, β, γ |
| N—Me | 30.7, CH₃ | 3.02, s | | α, N-MeAla⁵-CO |
| N-MeVal⁷ | | | | |
| CO | 172.6, C | | | |
| α | 59.8, CH | 5.18, d (10.7) | β | N—Me, β, γ, γ′, CO, N-MeVal⁶-CO |
| β | 28.8, CH | 2.32, m | α, γ, γ′ | α, γ, γ′ |
| γ | 18.6, CH₃ | 0.79, d (6.7) | β | α, β, γ′ |
| γ′ | 19.7, CH₃ | 0.90, o/l | β | α, β, γ |
| N—Me | 31.1, CH₃ | 3.01, s | | α, N-MeVal⁶-CO |
| N-MeVal⁸ | | | | |
| CO | 172.5, C | | | |
| α | 63.6, CH | 4.60, d (11.1) | β | N—Me, β, γ, γ′, CO |
| β | 27.6, CH | 2.23, o/l | α, γ, γ′ | α, γ, γ′ |
| γ | 18.9, CH₃ | 0.76, d (6.8) | β | α, β, γ′ |
| γ′ | 19.6, CH₃ | 0.92, o/l | β | α, β, γ |
| N—Me | 31.5, CH₃ | 3.11, s | | α, N-MeVal⁷-CO |
| N—Me | 26.1, CH₃ | 2.70, s | | N-MeVal⁸-CO |

^{a}Shift (125 MHz), type, determined by HSQC;
^{b}Shift (500 MHz), multiplicity, J (Hz);
^{c}overlapping signals;
^{d}multiplet

TABLE 9
Advanced Marfey's analysis of compound shagamide C (P5)

| | | Retention Time (min) | | | |
|---|---|---|---|---|---|
| Amino | | | | Shagamide C (5) | Absolute |
| Acids | m/z | D standard | L Standard | Hydrosylate | Configuration |
| Phe | 418.136 | 12.0 | 10.5 | 10.4 | L |
| N-MeAla | 356.120 | 37.7 | 38.1 | 38.1 | L |
| N-MeVal | 384.151 | 11.2 | 10.1 | 10.0 | L |
| N-MeLeu | 398.167 | 12.2 | 11.4 | 11.2 | L |

TABLE 10
Advanced Marfey's analysis of compound shagamide D (P6)

| | | Retention Time (min) | | | |
|---|---|---|---|---|---|
| Amino | | | | Shagamide D (6) | Absolute |
| Acids | m/z | D standard | L Standard | Hydrosylate | Configuration |
| Phe | 418.136 | 12.0 | 10.5 | 10.5 | L |
| N-MeAla | 356.120 | 37.7 | 38.1 | 38.0 | L |
| N-MeVal | 384.151 | 11.2 | 10.1 | 10.0 | L |
| N-MeLeu | 398.167 | 12.2 | 11.4 | 11.3 | L |

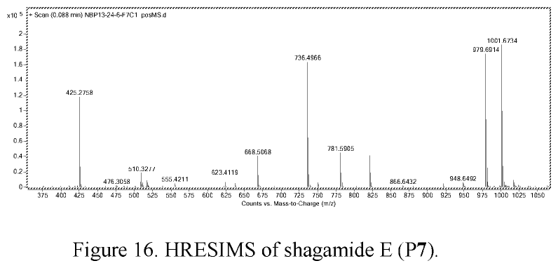

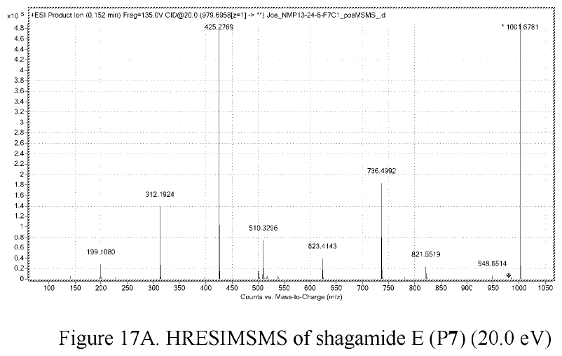

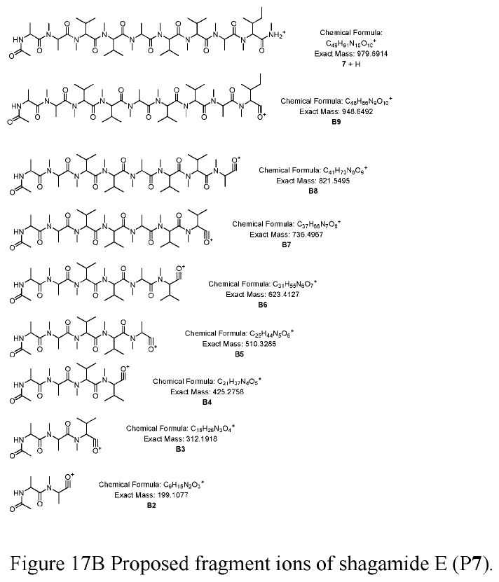

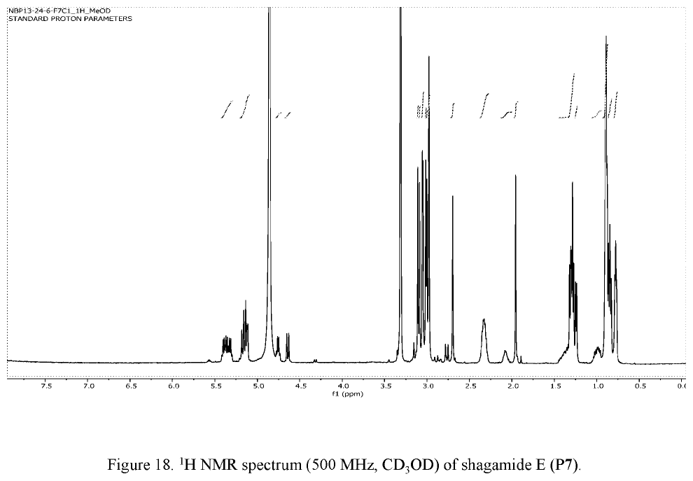

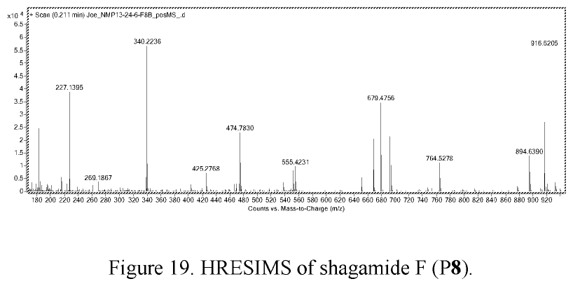

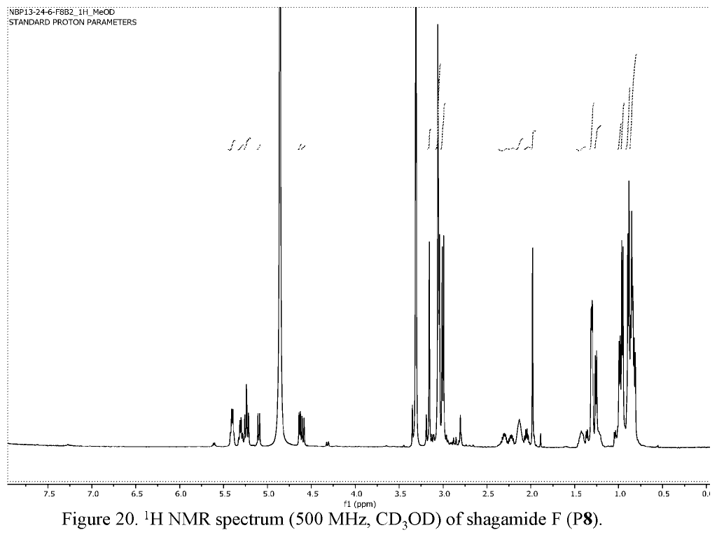

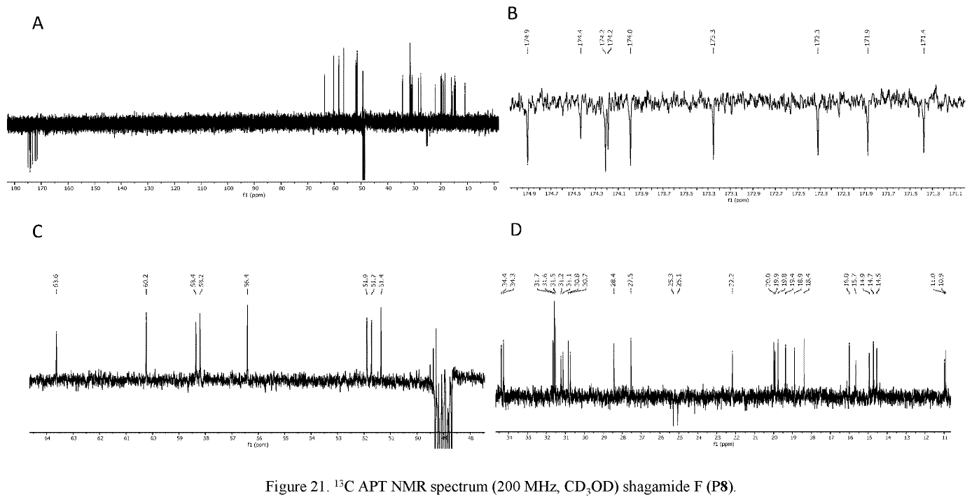

Shagamide E (P7) was isolated as a white solid, with the molecular formula of C₄₉H₉₀N₁₀O₁₀ deduced from the protonated molecule detected at m/z 979.6914 in the HRESIMS (FIG. 16). The amino acid sequence of Ala-N-MeAla-N-MeVal-N-MeVal-N-MeAla-N-MeVal-N-MeVal-N-MeAla-N-MeIle was determined from interpretation of the ¹H and ¹³C NMR data (Table 11), with a C-terminal N-methylamide based on MSMS fragmentation (FIG. 17A-B). The ¹H NMR spectrum (FIG. 18) showed a 3H singlet at δ_{H} 1.95, suggestive of an acetyl functionality. Indeed, an HMBC correlation from α-Ala¹ (δ_{H} 4.75) to the acetyl carbonyl (δ_{C} 172.8) confirmed the acetyl group to be placed at the N-terminus completing the planar structure of P7. Shagamide F (P8) was also isolated as a white solid, with a molecular formula of C₄₅H₈₃N₉O₉ inferred from the sodium adduct at m z 916.6205 detected in the HRESIMS (FIG. 19). The ¹H and ¹³C NMR spectra (FIGS. 20, 21) suggested this compound was also acetylated at the N-terminus, while analysis of the 2D NMR spectra deduced the amino acid sequence Val-N-MeAla-N-MeVal-N-MeAla-N-MeIle-N-MeIle-N-MeAla-N-MeVal with a primary amide at the C-terminus. Again, the L configuration for all amino acids in both P7 and P8 was determined using the advanced Marfey's method (Table 12 and Table 14, respectively).

TABLE 11
NMR data for shagamide E (P7) (CD₃OD)

| pos | δ_{C}, type^{a} | δ_{H}^{b} | gCOSY | gHMBC |
|---|---|---|---|---|
| Acetyl | | | | |
| CO | 172.9, C | | | |
| Me | 22.2, CH₃ | 1.95, s | | CO |
| Ala¹ | | | | |
| CO | 174.8, C | | | |
| α | 47.3, CH | 4.75, q (6.7) | β | β, CO, Acetyl-CO |
| β | 16.9, CH₃ | 1.28, d (6.7) | α | α, CO |
| N-MeAla² | | | | |
| CO | 174.6, CO | | | |
| α | 51.4, CH | 5.36, o/l^{c} | β | β, N—Me, CO |
| β | 14.6, CH₃ | 1.31, d (7.2) | α | α, CO |
| N-Me | 31.2, CH₃ | 3.11, s | | α, Ala¹-CO |
| N-MeVal³ | | | | |
| CO | 172.1, C | | | |
| α | 60.1, CH | 5.13, o/l | β | β, γ, γ′, N-Me, CO, Ala²-CO |
| β | 28.4, CH | 2.32, o/l | α, γ, γ′ | α, γ, γ′ |
| γ | 18.9, CH₃ | 0.84, o/l | β | α, β, γ′ |
| γ′ | 19.8, CH₃ | 0.89, o/l | β | α, β, γ |
| N-Me | 30.8, CH₃ | 3.01, s | | α, N-MeAla²-CO |
| N-MeVal⁴ | | | | |
| CO | 171.7, C | | | |
| α | 60.0, CH | 5.17, o/l | β | β, γ′, N-Me, CO |
| β | 28.5, CH | 2.32, o/l | α, γ, γ′ | α, γ, γ′ |
| γ | 18.4, CH₃ | 0.87, o/l | β | α, β, γ′ |
| γ′ | 20.0, CH₃ | 0.89, o/l | β | α, β, γ |
| N-Me | 31.0, CH₃ | 2.98, o/l | | α, Val³-CO |
| N-MeAla⁵ | | | | |
| CO | 174.9, C | | | |
| α | 51.8, CH | 5.32, o/l | β | β, N-Me, CO, Val⁴-CO |
| β | 15.0, CH₃ | 1.29, d (6.7) | α | α, CO |
| N-Me | 31.7, CH₃ | 3.08, s | | α, Val⁴-CO |
| N-MeVal⁶ | | | | |
| CO | 172.1, C | | | |
| α | 60.0, CH | 5.13, o/l | β | β, γ, γ′, N-Me, CO |
| β | 28.4, CH | 2.32, o/l | α, γ, γ′ | α, γ, γ′ |
| γ | 18.8, CH₃ | 0.84, o/l | β | α, β, γ′ |
| γ′ | 19.7, CH₃ | 0.89, o/l | β | α, β, γ |
| N-Me | 30.7, CH₃ | 3.05, s | | α, N-MeAla⁵-CO |
| N-MeVal⁷ | | | | |
| CO | 171.2, C | | | |
| α | 60.0, CH | 5.14, o/l | β | CO, N-Me, β, γ, γ′ |
| β | 28.4, CH | 2.32, o/l | α, γ, γ′ | α, γ, γ′ |
| γ | 18.4, CH₃ | 0.78, o/l | β | α, β, γ′ |
| γ′ | 20.0, CH₃ | 0.89, o/l | β | α, β, γ |
| N-Me | 31.0, CH₃ | 2.98, o/l | | α, Val⁶-CO |
| N-MeAla⁸ | | | | |
| CO | 174.1, C | | | |
| α | 51.8, CH | 5.39, o/l | β | β, N-Me, CO, Val⁷-CO |
| β | 14.5, CH₃ | 1.24, d (6.8) | α | α, CO |
| N-Me | 31.5, CH₃ | 3.00, s | | α, Val⁷-CO |
| N-MeIle⁹ | | | | |
| CO | 172.7, C | | | |
| α | 62.2, CH | 4.64, d (11.2) | β | β, γ, β-Me, N-Me, CO, N-MeAla⁸-CO |
| β | 33.5, CH | 2.07, m^{d} | α, β-Me | |
| β-Me | 15.7, CH₃ | 0.89, o/l | β | α, β, γ |
| γ | 25.8, CH₂ | 1.38, m | δ | |
| | | 1.00, m | δ | β-Me, β, δ |
| δ | 10.6, CH₃ | 0.87, o/l | γ | β, δ |
| N-Me | 31.3, CH₃ | 3.04, s | | α, N-MeAla⁸-CO |
| N-Me | 26.1, CH₃ | 2.70, s | | N-MeIle⁹-CO |

^{a}Shift (125 MHz), type, determined by HSQC;
^{b}Shift (500 MHz), multiplicity, J (Hz);
^{c}coverlapping signals;
^{d}multiplet

TABLE 12
Advanced Marfey's analysis of shagamide E (P7)

| | | Retention Time (min) | | | |
|---|---|---|---|---|---|
| | | | | Shagamide E (7) | Absolute |
| Amino Acids | m/z | D standard | L Standard | Hydrosylate | Configuration |
| Ala | 342.104 | 6.8 | 5.7 | 5.7 | L |
| N-MeAla | 356.120 | 37.7 | 38.1 | 38.1 | L |
| N-MeVal | 384.151 | 11.2 | 10.1 | 10.0 | L |
| N-MeIle | 398.167 | 12.8 | 11.6 | 11.6 | L |
| N-MeAlloIle | 398.167 | 13.1 | 11.9 | — | — |

TABLE 13
NMR data for shagamide F (P8) (CD₃OD)

| pos | δ_{C}, type^{a} | δ_{H}^{b} | gCOSY | gHMBC |
|---|---|---|---|---|
| Acetyl | | | | |
| CO | 173.3, C | 1.97, s | | |
| Me | 22.2, CH₃ | | | CO |
| Val¹ | | | | |
| CO | 174.0, C | | | |
| α | 56.4, CH | 4.63, d (7.6) | β | β, γ, γ′, CO, acetyl-CO |
| β | 31.6, CH | 2.06, m^{c} | α, γ, γ′ | α, γ, γ′ |
| γ | 18.4, CH₃ | 0.95, o/l^{d} | β | α, β, γ′ |
| γ′ | 19.8, CH₃ | 0.97, o/l | β | α, β, γ |
| N-MeAla² | | | | |
| CO | 174.2, C | | | |
| α | 51.4, CH | 5.40, q (7.3) | β | β, N-Me, CO |
| β | 14.7, CH₃ | 1.31, d (7.0) | α | α, CO |
| N-Me | 31.7, CH₃ | 3.16, s | | α, Val¹-CO |
| N-MeVal³ | | | | |
| CO | 171.9, C | | | |
| α | 60.2, CH | 5.10, d (10.7) | β | β, γ, γ′, N-Me, CO, N-MeAla²-CO |
| β | 28.4, CH | 2.30, m | α, γ, γ′ | α, γ, γ′ |
| γ | 18.9, CH₃ | 0.82, d (6.8) | β | α, β, γ′ |
| γ′ | 20.0, CH₃ | 0.89, o/l | β | α, β, γ |
| N-Me | 30.7, CH₃ | 2.99, s | | α, N-MeAla²-CO |
| N-MeAla⁴ | | | | |
| CO | 174.9, C | | | |
| α | 51.7, CH | 5.30, q (7.6) | β | β, N-Me, CO |
| β | 14.9, CH₃ | 1.30, o/l | α | α, CO |
| N-Me | 31.5, CH₃ | 3.06, s | | α, N-MeVal³-CO |
| N-MeIle⁵ | | | | |
| CO | 172.3, C | | | |
| α | 58.2, CH | 5.23, o/l | β | β, γ, β-Me, CO, N-Me, N-MeAla⁴-CO |
| β | 34.3, CH | 2.13, m | α, β-Me | |
| β-Me | 15.7, CH₃ | 0.85, o/l | β | α, β, γ |
| γ | 25.3, CH₂ | 1.43, m | δ | β, γ |
| | | 0.99, o/l | δ | β, γ |
| δ | 11.0, CH₃ | 0.87, o/l | γ | β, δ |
| N-Me | 31.6, CH₃ | 3.06, s | | α, Ala⁴-CO |
| N-MeIle⁶ | | | | |
| CO | 171.4, C | | | |
| α | 58.4, CH | 5.25, o/l | β | β, β-Me, CO, N-Me, N-MeIle⁵-CO |
| β | 34.4, CH | 2.13, o/l | α, β-Me | |
| β-Me | 16.0, CH₃ | 0.85, o/l | β | α, β, γ |
| γ | 25.1, CH₂ | 1.22, o/l | δ | |
| | | 0.97, o/l | δ | |
| δ | 10.9, CH₃ | 0.88, o/l | γ | β, δ |
| N-Me | 31.2, CH₃ | 3.01, s | | α, N-MeIle⁵-CO |
| N-MeAla⁷ | | | | |
| CO | 174.2, C | | | |
| α | 51.9, CH | 5.40, o/l | β | β, N-Me, CO |
| β | 14.5, CH₃ | 1.26, d (6.8) | α | α, CO |
| N-Me | 30.8, CH₃ | 3.05, s | | α, N-MeIle⁶-CO |
| N-MeVal⁸ | | | | |
| CO | 174.4, C | | | |
| α | 63.6, CH | 4.59, d (11.0) | β | β, γ, γ′, N-Me, CO |
| β | 27.5, CH | 2.22, m | α, γ, γ′ | α, γ, γ′ |
| γ | 19.4, CH₃ | 0.84, o/l | β | α, β, γ′ |
| γ′ | 19.9, CH₃ | 0.99, o/l | β | α, β, γ |
| N-Me | 31.1, CH₃ | 3.04, s | | α, N-MeAla⁷-CO |

^{a}Shift (125 MHz), type, determined by HSQC;
^{b}Shift (500 MHz), multiplicity, J (Hz);
^{c}multiplet;
^{d}overlapping signals

TABLE 14
Advanced Marfey's analysis of shagamide F (P8)

| | | Retention Time (min) | | | |
|---|---|---|---|---|---|
| | | | | Shagamide F | |
| | | | | (8) | Absolute |
| Amino Acids | m/z | D standard | L Standard | Hydrosylate | Configuration |
| Val | 370.135 | 10.3 | 8.4 | 8.3 | L |
| N-MeAla | 356.120 | 37.7 | 38.1 | 38.0 | L |
| N-MeVal | 384.151 | 11.2 | 10.1 | 9.9 | L |
| N-MeIle | 398.167 | 12.8 | 11.6 | 11.6 | L |
| N-MeAlloIle | 398.167 | 13.1 | 11.9 | — | — |

Structurally, the friomaramides and shagamides join a rare class of natural product, each N-methylated across every peptide bond in the backbone. The N-termini of RiPPs and NRPs can be made up of a variety of amide caps, with acetyl groups frequently observed in both. Formyl groups have previously been observed appended to marine invertebrate-sourced peptides,²¹⁻²⁸ and provide evidence that shagamides have a NRPs biogenesis as no formylated RiPPs have been previously reported.²⁹ The mono-substituted ureido moiety present at the N-terminus of shagamide D (P6) is extremely rare. To date, the cyanobacterial cyclic peptide anabaenopeptin 679 is the only previously reported peptidic natural product to possess this moiety,³⁰ and shagamide D is the first reported example with a mono-substituted ureido cap at its N-terminus.

TABLE 15
NMR data for friomaramide A (P1) (CD₃OD)

| pos | δ_{C}, type^{a} | δ_{H}^{b} | gCOSY | gHMBC |
|---|---|---|---|---|
| Acetyl | | | | |
| CO | 172.7, C | | | |
| CH3 | 22.1, CH₃ | 1.93, s | | CO |
| Phe1 | | | | |
| CO | 174.0, C | | | |
| α | 52.1, CH | 5.15, dd (8.9, 6.6) | β | β, CO, acetyl-CO |
| β | 39.0, CH₂ | 2.92, m^{d} | α | α, γ, δ, CO |
| | | 3.06, m | α | α, γ, δ |
| γ | 138.0, C | | | |
| δ | 130.4, CH | 7.25, o/l^{c} | ε | ε |
| ε | 129.6, CH | 7.27, o/l | δ, ζ | γ |
| ζ | 128.0, CH | 7.23, o/l | ε | δ |
| N-MeVal2 | | | | |
| CO | 171.5, C | | | |
| α | 60.0, CH | 5.04, d (10.7) | β | N-Me, β, γ, γ′, CO, Phe¹-CO |
| β | 28.5, CH | 2.23, m | α, γ, γ′ | α |
| γ | 18.5, CH₃ | 0.78, o/l | β | α, β, γ′ |
| γ′ | 19.9, CH₃ | 0.82, o/l | β | α, β, γ |
| N-Me | 30.7, CH₃ | 2.87, s | | α, Phe¹-CO |
| N-MeAla3 | | | | |
| CO | 174.7, C | | | |
| α | 51.4, CH | 5.19 (q, 7.1) | β | β, N-Me, CO |
| β | 15.2, CH₃ | 1.20, d (7.1) | α | α, CO |
| N-Me | 31.5, CH₃ | 2.78, s | | α, N-MeVal²-CO |
| N-MeIle4 | | | | |
| CO | 171.7, C | | | |
| α | 58.5, CH | 5.10, d (10.9) | β | β, γ, β-Me, N-Me, CO, Ala³-CO |
| β | 34.1, CH | 1.99, m | α, β-Me | |
| β-Me | 15.6, CH₃ | 0.62, d (6.6) | β | α, β, γ |
| γ | 25.3, CH₂ | 0.86, m | δ | |
| δ | 10.9, CH₃ | 0.79, o/l | γ | β, γ |
| N-Me | 30.7, CH₃ | 2.96, s | | α, N-MeAla³-CO |
| N-MeIle5 | | | | |
| CO | 172.2, C | | | |
| α | 59.3, CH | 5.35, d (11.0) | | β, γ, β-Me, N-Me, CO |
| β | 34.4, CH | 2.10, m | | |
| β-Me | 16.0, CH₃ | 0.77, d (6.5) | | α, β, γ |
| γ | 24.9, CH₂ | 0.91, o/l | | |
| δ | 11.1, CH₃ | 0.83, o/l | | β, γ |
| N-Me | 31.3, CH₃ | 3.02, s | | α, N-MeIle⁴-CO |
| N-MeTrp-ene | | | | |
| 1 | 124.5, CH | 6.33, d (8.2) | 1 | 2, 3′ |
| 2 | 120.8, CH | 6.58, d (8.3) | 2 | 1, 2′, 3a′ |
| 2′ | 125.9, CH | 7.22, s | | 3′, 3a′, 7a′ |
| 3′ | 110.5, C | | | |
| 3a′ | 128.2, C | | | |
| 4′ | 119.4, CH | 7.60, dt (8.0, 1.0) | 5′ | 3′, 6′, 7a′ |
| 5′ | 120.9, CH | 7.09, ddd (8.0, | 4′, 6′ | 3a′, 7′ |
| | | 7.0, 1.0) | | |
| 6′ | 123.2, CH | 7.15, ddd (8.1, | 5′, 7′ | 4′, 7a′ |
| | | 7.0, 1.1) | | |
| 7′ | 112.5, CH | 7.37, dt (8.2, 0.9) | 6′ | 3a′, 5′ |
| 7a′ | 137.4, C | | | |
| N-Me | 34.2 CH₃ | 3.08, s | | 1, N-MeIle⁵-CO |

^{a}Shift (200 MHz), type, determined by HSQC;
^{b}Shift (800 MHz), multiplicity, J (Hz);
^{c}overlapping signals;
^{d}multiplet

TABLE 16
Advanced Marfey's analysis of friomaramide A (P1)

| | | Retention Time (min) | | | |
|---|---|---|---|---|---|
| | | | | Friomaramide A | Absolute |
| Amino Acids | m/z | D standard | L Standard | (1) Hydrosylate | Configuration |
| Phe | 418.136 | 12.1 | 10.7 | 10.6 | L |
| N-MeVal | 384.151 | 11.2 | 10.2 | 10.2 | L |
| N-MeAla | 356.120 | 38.4 | 38.9 | 38.8 | L |
| N-MeLeu | 398.167 | 12.4 | 11.6 | — | L |
| N-MeIle | 398.167 | 13.1 | 11.9 | 11.9 | L |
| N-MeAlloIle | 398.167 | 13.3 | 12.1 | — | — |

### Example 2

### Novel Compounds Reduce Growth and Viability of Plasmodium.

Based on the previous observed potency of friomaramide A against blood-state Plasmodium falciparum, all newly isolated compounds were subjected to an assessment for anti-malarial potential, including both sensitive (NF54 and 3D37) and resistant (Dd2) P. falciparum strains in the blood stage (Table 17A), as well as cytotoxicity profiling (Table 17B). To ensure our compounds were selective for the P. falciparum and the results were not just simply due to cell death, the newly isolated compounds were assayed against the murine macrophage J774 cell line, where none exhibited significant cytotoxic properties as all have IC₅₀ value >20 μM.

TABLE 17A
IC₅₀ Values (μM) of Metabolites 2-8 against
Blood-Stage P. falciparum Strains

| Compound | NF54 | Dd2 | 3D7 |
|---|---|---|---|
| P2 | 3.93 | 3.30 | 8.65 |
| P3 | 2.61 | 1.72 | 2.10 |
| P4 | >10 | 4.37 | >10 |
| P5 | 3.73 | 1.07 | 2.52 |
| P6 | 3.05 | 2.02 | >10 |
| P7 | >10 | >10 | >10 |
| P8 | >10 | >10 | >10 |
| chloroquine | 0.03 | 1.28 | 0.03 |

TABLE 17B
Friomaramide B (P2) and Shagamides A-F (P2-P8) cytotoxicity
testing against murine J774 macrophage cells

| Compound | EC₅₀ (μg/mL) | EC₅₀ (μM) |
|---|---|---|
| Friomaramide B (P2) | 26.7 | 24.6 |
| Shagamide A (P3) | >100 | >100 |
| Shagamide A (P4) | >100 | >100 |
| Shagamide A (P5) | >100 | >100 |
| Shagamide A (P6) | >100 | >100 |
| Shagamide A (P7) | >100 | >100 |
| Shagamide A (P8) | >100 | >100 |
| Chloroquine | 16.3 | 51.0 |

From the initial blood-stage screen against several P. falciparum strains, P2, P3, P5 and P6 demonstrated viable potential with activity below 10 μg/mL (<10 μM) against multiple strains, and these metabolites were prioritized for further investigation (Table 17A). From this data, several key structure-activity relationship (SAR) features were realized. First, the rare tryptenamine C-terminal residue of friomaramide A (P1) is not essential to activity. Although tryptenamine-bearing friomaramide B (P2) had modest activity, shagamides P3, P5 and P6, which have a hydrophobic valine or isoleucine residue C-terminus instead of tryptenamine, were generally as active or more active than friomaramide A. This suggests the mechanism of anti-malarial activity is not dependent on the trypteneamine and will simplify future synthetic efforts in lead compound development. Methylation of the C-terminus amide does not appear to affect activity (vis P3, active but lacking C-terminus methylation). Next, it appears essential that the N-terminal amino acid residue is phenylalanine. The compounds that did not show significant activity (P4, P7 and P8) all have the small hydrophobic amino acids alanine or valine in place of this residue, and this likely abrogates the observed activity. The four most active compounds have did have the full variety of N-terminal amide caps that were isolated. While P5 and P6 showed similar potency against NF54 and Dd2, P5 demonstrated significant inhibition of strain 3D7 (2.52 μg/mL) and P6 was inactive, suggesting the formyl moiety is more efficacious than the primary-ureido moiety as these two compounds only differ by this amide cap. The only other metabolite with comparable activity against the 3D7 strain was P3 which also possessed a formyl cap, however alone this is not sufficient for activity (formylated metabolite P4 is inactive) thus providing a potential lead for future synthetic efforts. These metabolites, P2-P6, demonstrated promising activity against the drug-resistant strain Dd2, therefore with structure optimization may provide a novel mechanism of action although these studies are currently ongoing in our lab.

### Example 3

### Extraction and Isolation of Novel Peptides.

Optical rotations were measured using an AutoPol IV polarimeter at 589 nm. UV/Vis spectra were extracted from HPLC chromatograms. NMR spectra were acquired using either a Varian Inova 500 spectrophotometer or a Bruker Bio-Spin 800 MHz spectrophotometer equipped with a 5 mm TXI cryoprobe. The residual solvent peak was used as an internal chemical shift reference (CD₃OD: δ_{C} 49.0; δ_{H} 3.31, DMSO-d₆: δ_{C} 39.52; δ_{H} 2.50). High-resolution mass spectrometry/liquid chromatography data were obtained on an Agilent 6540 QTOF LCMS with electrospray ionization detection. Medium-pressure liquid chromatography (MPLC) was performed using a Combiflash Rf 200i MPLC, using ELSD and UV detection with a RediSepRf 80 g silica column. Reversed-phase HPLC was performed on a Shimadzu LC20-AT system equipped with a photodiode array detector (M20A) using a semipreparative Phenominex C18 column (10 μm, 100 Å, 250×10 mm; 4 mL/min) or on an analytical Phenomenex polar C18 column (5 μm, 100 Å, 250×4.6 mm; 1 mL/min). All solvents used for column chromatography were of HPLC grade, and H₂O was distilled. Solvent mixtures are reported as % v/v unless otherwise stated.

The Inflatella coelosphaeroides specimens and their identification used for this study were described in detail in our previous study.²¹ The remaining sponge fractions of interest generated in the initial study were combined and analyzed by LCMS. Briefly, a collection of I. coelosphaeroides sponge samples were extracted twice overnight in 1:1 CH₂Cl₂/MeOH. The extracts were dried and mounted onto silica gel, and then partitioned using NP MPLC with a gradient from hexane to EtOAc and washed with 20% MeOH in EtOAc. Friomaramide A (P1) was isolated from the E fraction eluting at 20% MeOH in EtOAc, so the remaining E side-fractions alongside those of F were combined to generate ‘Extract 2021’. Extract 2021 (70 mg) was fractionated with semi-preparative C18 HPLC (4 mL/min), using a linear gradient from 10% MeOH/H₂O to 100% MeOH over 60 min, generating fractions 1-16. The individual fractions were then purified again by C18 HPLC (4 mL/min) this time using a linear gradient from 10% CAN/H₂O to 100% CAN over 60 min, and finally cleaned by analytical polar C18 HPLC (1 mL/min) using a linear gradient from 50% MeOH/H₂O to 100% MeOH over 60 min to afford the pure peptides, friomaramide B (0.60 mg), shagamide A (1.05 mg), shagamide B (0.80 mg), shagamide C (1.15 mg), shagamide D (1.05 mg), shagamide E (1.40 mg) and shagamide F (1.25 mg).

Friomaramide B (P2): white solid; [α]²²_{D} −17.4 (c 0.05, MeOH); UV (MeOH) λₘₐₓ 221, 280 nm; ¹H and ¹³C NMR (CD₃OD) Table 1; HRESIMS m/z 1086.6729 [M+H]⁺ (calcd for C₆₂H₈₈N₉O₈, 1086.6750; Δ−1.93 ppm).

Shagamide A (P3): white solid; [α]²²_{D} −151.4 (c 0.05, MeOH); UV (MeOH) ma 215, 275 nm; ¹H and ¹³C NMR, see Table 3; HRESIMS m/z 743.4472 [M+Na]⁺ (calcd for C₄₀H₆₀N₆O₆Na, 743.4467; Δ0.67 ppm).

Shagamide B (P4): white solid; [α]²²_{D} −66.6 (c 0.05, MeOH); UV (MeOH) λₘₐₓ 220, 280 nm; ¹H and ¹³C NMR, see Table 5; HRESIMS m/z 859.6015 [M+Na]⁺ (calcd for C₄₃H₈₀N₈O₈Na, 859.5991; Δ2.79 ppm).

Shagamide C (P5): white solid; [α]²²_{D} −196.6 (c 0.05, MeOH); UV (MeOH) λₘₐₓ 215 nm; ¹H and ¹³C NMR, see Table 7; HRESIMS m/z 978.6364 [M+Na]⁺ (calcd for C₅₀H₅₅N₉O₉Na, 978.6362; Δ0.20 ppm).

Shagamide D (P6): white solid; [α]²²_{D} −166.6 (c 0.05, MeOH); UV (MeOH) λₘₐₓ 215, 275 nm; ¹H and ¹³C NMR, see Table 8; HRESIMS m/z 971.6666 [M+H]⁺ (calcd for C₅₀H₈₇N₁₀O₉, 971.6652; Δ1.44 ppm).

Shagamide E (P7): white solid; [α]²²_{D} −214.0 (c 0.05, MeOH); UV (MeOH) λₘₐₓ 215 nm; ¹H and ¹³C NMR, see Table 11; HRESIMS m/z 979.6914 [M+H]⁺ (calcd for C₄₉H₉₁N₁₀O₁₀, 979.6914; Δ0 ppm).

Shagamide F (P8): white solid; [α]²²_{D} −150.6 (c 0.05, MeOH); UV (MeOH) λₘₐₓ 215, 275 nm; ¹H and ¹³C NMR, see Table 13; HRESIMS m/z 916.6205 [M+H]+(calcd for C₄₅H₈₃N₉O₉Na, 916.6206; Δ0.11 ppm).

### Example 4

### Stereochemical Analysis of Novel Compounds Using the Advanced Marfey's Method.

Δ0.1 mg sample of peptides P2-P8 was individually hydrolyzed with 200 μL of 6 N HCl at 100° C. for 12 h. The hydrolysate was then evaporated to dryness under a stream of N₂ overnight, and then lyophilized for 1 h to remove residual HCl. It was then suspended in 100 μL of H₂O and treated with 50 μL of sat. NaHCO₃ (aq) and 200 μL of 1% 1-fluoro-2-4-dinitrophenyl-5-L-alanine amide (FDAA) in acetone. The solution was stirred at 40° C. for 1 h before being quenched with 50 μL of 1M HCl.

Next 250 μL of ACN was added and it was passed through a syringe filter and analyzed by LCMS.

Δ1 μL injection of the solution was run on a Kinetex C18 column (2.6 μm, 100 Å, 150×3 mm; 0.5 mL/min) using two methods individually. First, a linear gradient from 25% ACN/H₂O (0.1% HCO₂H) to 100% ACN/H₂O (0.1% HCO₂H) over 40 min, and second an isocratic elution of 20% ACN/H₂O (0.1% HCO₂H, 5% ACN) for 20 min followed by a linear gradient to 40% ACN/H₂O (0.1% HCO₂H, 5% ACN) over 20 min (to get separation of N-MeAla isomers). The amino acid content of the hydrolysate was assessed by monitoring the (+)-HRESIMS and the EIC retention times were compared to standards synthesized and analyzed by the same method.

### Example 5

### Cytotoxic Analysis of Novel Compounds in Mammalian Cells.

The isolated compounds were each tested for cytotoxicity using mammalian J774A.1 cell lines (ATCC TIB-67™) in complete media; RPMI 1640 medium with phenol red containing L-glutamine and then supplemented with 10% fetal bovine serum (CM). Cells were seeded at 5×10⁵ cell/mL and plated in a 96-well format with 100 μL/well fresh media on day 0 and were incubated overnight at 37° C., 5% CO₂ for adherence. Following 24 h incubation, spent media was removed and 100 μL of fresh media with test compounds were serially diluted 1:2 with a starting concentration of 10 μg/mL was added to the cells and incubated for an additional 68 h before adding in 20 μL CellTiter 96® AQueous One Solution Cell Proliferation Assay reagent (Promega). Cells were incubated for an additional 4 h before reading absorbance (490) nm on the CLARIOstar plate reader.

### Example 6

### Antimalarial Analysis of Novel Compounds Via a Blood-Stage Antimalarial Assay.

For parasite suspension, P. falciparum malaria strains were grown under normal conditions as previously described by Trager and Jensen with some modifications.” Briefly, parasites were suspended in RPMI 1640 supplemented with 0.23% sodium bicarbonate, 50 mg/mL hypoxanthine and 0.5% albumax (CM), 4% packed washed red blood cells by volume and incubated in a continuous gas incubator set to normal conditions (5% O₂, 5% CO₂, 95% N). Parasites were prepared for drug susceptibility assay by highly synchronizing schizonts 16 h before experiment start by using a 70% percoll method. Ring stage parasites were then resuspended in culture with fresh CM containing 2% hematocrit and 0.5% parasitemia.

Antimalaria activity was assessed against reference strains of NF54, 3D7 and Dd2 using an adaptation of the sensitivity assay of Desjardins et al. using SybrGreen fluorescence as an assessment of parasite growth.³² NF54 original isolate was obtained from a patient living near Schiphol Airport, Amsterdam and its clonal isolate 3D7 are generally considered to be drug sensitive, though 3D7 does convey resistance to sulfadoxine. Dd2 derived from the parent W2, is multi-drug-resistant line originating from the Indochina III/CDC isolate, which contain point mutations in pfcrt as well as amplifications in pfmdr1 and GTP cyclohydrolase. Briefly, compounds were diluted to 10 μg/mL and serially diluted along a microtiter plate before adding in 100 μL/well of the parasite suspension. Parasite growth was compared to controls incubated at 37° C. and normal culture conditions (5% O₂, 5% CO₂, 90% N₂) for 72 h. Plates were frozen and thawed before adding a final 1× concentration of SybrGreen in lysis buffer and read on the CLARIOstar fluorescence plate reader (Ex/Em 484-15/528-15) following a 45 min incubation at room temperature. Drug EC₅₀ values were calculated by interpolation of the probit transformation of the log(dose)-response curve using the MARS software provided by BMG.

### LIST OF REFERENCES

1. WHO World Malaria Report 2021. https://www.who.int/teams/global-malaria-programme/reports/world-malaria-report-2021.
2. Jagannathan, P.; Kakuru, A., Malaria in 2022: Increasing challenges, cautious optimism. Nature Commun. 2022, 13, 2678.
3. Tindana, P.; de Haan, F.; Amaratunga, C.; Dhorda, M.; van der Pluijm, R. W.; Dondorp, A. M.; Cheah, P. Y., Deploying triple artemisinin-based combination therapy (TACT) for malaria treatment in Africa: ethical and practical considerations. Malaria J. 2021, 20, 119.
4. Owoloye, A.; Olufemi, M.; Idowu, E. T.; Oyebola, K. M., Prevalence of potential mediators of artemisinin resistance in African isolates of Plasmodium falciparum. Malaria J. 2021, 20, 451.
5. Fidock, D. A.; Rosenthal, P. J., Artemisinin resistance in Africa: How urgent is the threat? Med 2021, 2, 1287-1288.
6. Tajuddeen, N.; Van Heerden, F. R., Antiplasmodial natural products: an update. Malaria J. 2019, 18, 404.
7. Hai, Y.; Cai, Z.-M.; Li, P.-J.; Wei, M.-Y.; Wang, C.-Y.; Gu, Y.-C.; Shao, C.-L., Trends of antimalarial marine natural products: progresses, challenges and opportunities. Nat. Prod. Rep. 2022, 39, 969-990.
8. Aguiar, A. C. C.; Parisi, J. R.; Granito, R. N.; de Sousa, L. R. F.; Renno, A. C. M.; Gazarini, M. L., Metabolites from marine sponges and their potential to treat malarial protozoan parasites infection: A systematic review. Mar. Drugs 2021, 19, 134.
9. Lebar, M. D.; Heimbegner, J. L.; Baker, B. J., Cold-water marine natural products. Nat. Prod. Rep. 2007, 24, 774-797.
10. Soldatou, S.; Baker, B. J., Cold-water marine natural products, 2006 to 2016. Nat. Prod. Rep. 2017, 34, 585-626.
11. Núñez-Ponz, L.; Shilling, A. J.; Verde, C.; Baker, B. J.; Giordano, D., Marine terpenoids from polar latitudes and their potential applications in biotechnology. Mar. Drugs 2020, 18, 401.
12. Núñez-Pons, L.; Avila, C., Natural products mediating ecological interactions in Antarctic benthic communities: a mini-review of the known molecules. Nat. Prod. Rep. 2015, 32, 1114-1130.
13. Carroll, A. R.; Copp, B. R.; Davis, R. A.; Keyzers, R. A.; Prinsep, M. R., Marine natural products. Nat. Prod. Rep. 2021, 38, 362-413.
14. Shilling, A. J.; Witowski, C. G.; Maschek, J. A.; Azhari, A.; Vesely, B.; Kyle, D. E.; Amsler, C. D.; McClintock, J. B.; Baker, B. J., Spongian diterpenoids derived from the Antarctic sponge Dendrilla antarctica are potent inhibitors of the Leishmania parasite. J. Nat. Prod. 2020, 83, 1553-1562.
15. Li, F.; Pandey, P.; Janussen, D.; Chittiboyina, A. G.; Ferreira, D.; Tasdemir, D., Tridiscorhabdin and didiscorhabdin, the first discorhabdin oligomers linked with a direct C—N bridge from the sponge Latrunculia biformis collected from the deep sea in Antarctica. J. Nat. Prod. 2020, 83, 706-713.
16. Urda, C.; Pérez, M.; Rodríguez, J.; Jimenez, C.; Cuevas, C.; Fernández, R., Pembamide, a N-methylated linear peptide from a sponge Cribrochalina sp. Tetrahedron Lett. 2016, 57, 3239-3242.
17. Boot, C. M.; Tenney, K.; Valeriote, F. A.; Crews, P., Highly N-methylated linear peptides produced by an atypical sponge-derived Acremonium sp. J. Nat. Prod. 2006, 69, 83-92.
18. Dewapriya, P.; Khalil, Z. G.; Prasad, P.; Salim, A. A.; Cruz-Morales, P.; Marcellin, E.; Capon, R. J., Talaropeptides A-D: Structure and biosynthesis of extensively N-methylated linear peptides from an Australian marine tunicate-derived Talaromyces sp. Front. Chem. 2018, 6.
19. Nabika, R.; Oishi, S.; Misu, R.; Ohno, H.; Fujii, N., Synthesis of IB-01212 by multiple N-methylations of peptide bonds. Bioorg. Med. Chem. 2014, 22, 6156-6162.
20. Biron, E.; Chatterjee, J.; Ovadia, O.; Langenegger, D.; Brueggen, J.; Hoyer, D.; Schmid, H. A.; Jelinek, R.; Gilon, C.; Hoffman, A.; Kessler, H., Improving oral bioavailability of peptides by multiple N-methylation: Somatostatin analogues. Angew. Chem. Internat. Ed. 2008, 47, 2595-2599.
21. Knestrick, M. A.; Wilson, N. G.; Roth, A.; Adams, J. H.; Baker, B. J., Friomaramide, a highly modified linear hexapeptide from an Antarctic sponge, inhibits Plasmodium falciparum liver-stage development. J. Nat. Prod. 2019, 82, 2354-2358.
22. Izumikawa, M.; Hashimoto, J.; Takagi, M.; Shin-ya, K., Isolation of two new terpeptin analogs-JBIR-81 and JBIR-82-from a seaweed-derived fungus, Aspergillus sp. SpD081030G1f1. J. Antibiot. 2010, 63, 389-391.
23. Ondeyka, J. G.; Dombrowski, A. W.; Polishook, J. P.; Felcetto, T.; Shoop, W. L.; Guan, Z.; Singh, S. B., Isolation and insecticidal activity of mellamide from Aspergillus melleus. J. Ind. Microbiol. Biotechnol. 2003, 30, 220-224.
24. Palermo, J. A.; Flower, P. B.; Seldes, A. M., Chondriamides A and B, new indolic metabolites from the red alga Chondria sp. Tetrahedron Lett. 1992, 33, 3097-3100.
25. Harada, K.-i.; Fujii, K.; Mayumi, T.; Hibino, Y.; Suzuki, M.; Ikai, Y.; Oka, H., A method using LCMS for determination of absolute configuration of constituent amino acids in peptide—advanced Marfey's method. Tetrahedron Lett. 1995, 36, 1515-1518.
26. Vijayasarathy, S.; Prasad, P.; Fremlin, L. J.; Ratnayake, R.; Salim, A. A.; Khalil, Z.; Capon, R. J., C₃ and 2D C₃ Marfey's methods for amino acid analysis in natural products. J. Nat. Prod. 2016, 79, 421-427.
27. Fukuhara, K.; Takada, K.; Okada, S.; Matsunaga, S., Nazumazoles D-F, cyclic pentapeptides that inhibit chymotrypsin, from the marine sponge Theonella swinhoei. J Nat. Prod. 2016, 79, 1694-1697.
28. Kobayashi, J.; Cheng, J.; Ohta, T.; Nakamura, H.; Nozoe, S.; Hirata, Y.; Ohizumi, Y.; Sasaki, T., Iejimalides A and B, novel 24-membered macrolides with potent antileukemic activity from the Okinawan tunicate Eudistoma cf. rigida. J. Org. Chem. 1988, 53, 6147-6150.
29. Wenski, S. L.; Thiengmag, S.; Helfrich, E. J. N., Complex peptide natural products: Biosynthetic principles, challenges and opportunities for pathway engineering. Synth. Syt. Biotechnol. 2022, 7, 631-647.
30. Harms, H.; Kurita, K. L.; Pan, L.; Wahome, P. G.; He, H.; Kinghorn, A. D.; Carter, G. T.; Linington, R. G., Discovery of anabaenopeptin 679 from freshwater algal bloom material: Insights into the structure-activity relationship of anabaenopeptin protease inhibitors. Bioorg. Med. Chem. Lett. 2016, 26, 4960-4965.
31. Trager, W.; Jensen, J. B., Human malaria parasites in continuous culture. Science 1976, 193, 673-675.
32. Desjardins, R. E.; Canfield, C. J.; Haynes, J. D.; Chulay, J. D., Quantitative assessment of antimalarial activity in vitro by a semiautomated microdilution technique. Antimicrob. Agents Chemother. 1979, 16, 710-718.

### Example 7

Isolation and Characterization of Bioreactive Compounds from Antarctic Deep-Water Octocoral Alcyonium sp.

Corals are encountered from the tropics to the polar seas, found on seamounts or geological formations up to 6000 m below the ocean's surface [1,2]. In the south, corals are separated by the Antarctic Circumpolar Current from the contiguous oceans resulting in an ecological niche [3,4]. Biochemical knowledge of deep-water corals from Antarctica is impeded by the remoteness and extreme conditions required for access [5,6], leading to great interest in natural coral products for ecological and biomedical studies [7,8,9]. Past research suggests that deep-water coral species offer potential drug discovery resources from the terpenoids class, ranging from mono- to triterpenes [10,11,12]. Various cold-water terpenoids from deep-sea soft corals include the paesslerins [13], ainigmaptilones [14], and keikipukalides [6], many of which exhibit moderate cytotoxicity toward either human cancer cell lines or microbial pathogens [8].

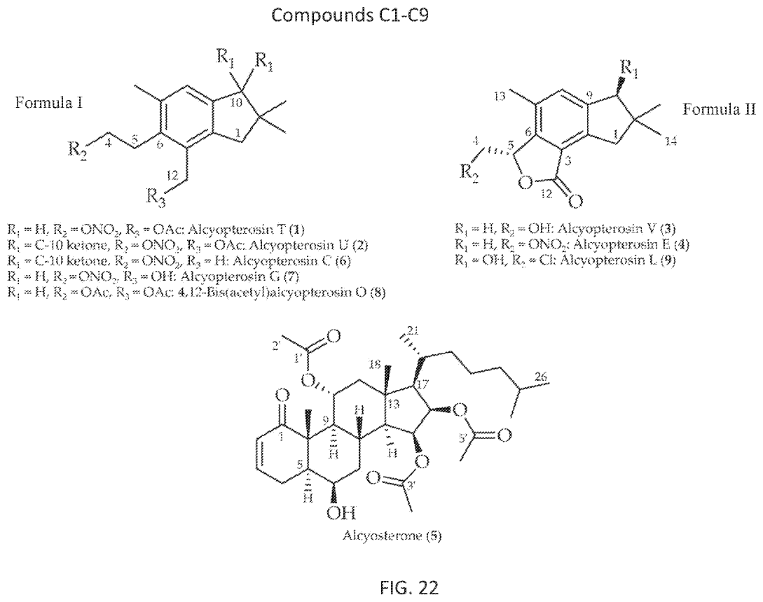

Originally found in fungi [15], illudalane sesquiterpenes have also been isolated from deep-sea corals [16,17] and marine sedimentary fungi [18]. Alcyopterosins are illudalane metabolites reported from the Antarctic soft corals Alcyonium paessleri and A. grandis that display terminal chlorine, hydroxyl, or nitrate ester moieties at the C-4 position of the aliphatic side chain [16,17]. Nitrate in seawater is considerably less abundant than, for example, the halides, so the appearance of a nitrate ester is unexpected and, to date, found exclusively in this class of marine natural products. We had the opportunity to study Alcyonium sp. from deep-water communities near Shag Rocks in the Scotia Arc of Antarctica. Six known alcyopterosins and three new ones (C1-C3) were obtained, in addition to a highly oxidized steroid, alcyosterone (C5) (FIG. 22). The metabolites were screened in a number of anti-infective assays and several showed promise against Clostridium difficile and Leishmania donovani.

Coral specimens were collected during a 2013 cruise to the Scotia Arc in the Southern Ocean near Shag Rocks, at a depth of between 126 and 130 m. Phylogenetic analysis was conducted on one specimen (WAM Z97931) using the msh1 sequence. The coral clustered with other known Alcyonium spp. from the Southern Ocean region, but was divergent from those species (FIG. 27), leading to its current identification as Alcyonium sp. indet.

The dichloromethane/methanol (1:1) extract of the freeze-dried coral was partitioned between ethyl acetate and water, and the lipophilic partition was separated using a gradient normal-phase medium pressure liquid chromatography (MPLC) system, yielding eight fractions. Several MPLC fractions were chosen for HPLC purification based on the characteristics of their ¹H NMR spectra. In particular, the mid- and late-polar fractions displayed ¹H NMR signals characteristic of the previously reported alcyopterosins [16,17], in particular the aromatic singlet (H-8) and a midfield oxymethylene (H₂-4). Fractions F, G, and H, eluting roughly between 60-90% ethyl acetate in hexane, were found to harbor alcyopterosins E (C4), C (C6), G (C7), 4,12-bis(acetyl)alcyopterosin O (C8), and alcyopterosin L (C9) (FIGS. 22, 51-52). Two new alcyopterosins (C1, C2) were found in the earlier eluting MPLC fractions, D and E, and fraction H was found to contain the previously undescribed hydrolysis product (C3) of alcyopterosin E (C4), along with C4.

TABLE 18
NMR shift comparison between compounds isolated in
the current work to those previously published.

| | Alcyopterosin E | | | | Alcyopterosin L | | | | Alcyopterosin C | | | |
|---|---|---|---|---|---|---|---|---|---|---|---|---|
| | This work | | Palermo¹⁶ | | This work | | Palermo¹⁶ | | This work | | Palermo¹⁶ | |
| Position | δ_{H} | δ_{C} | δ_{H} | δ_{C} | δ_{H} | δ_{C} | δ_{H} | δ_{C} | δ_{H} | δ_{C} | δ_{H} | δ_{C} |
| 1 | 3.03 | 44.7 | 3.04 | 45 | 2.92 | 44.0 | 2.92 | 44.0 | 2.87 | 42.3 | 2.87 | 42.3 |
| | | | | | 3.17 | | 3.17 | | | | | |
| 2 | | 148 | | 148 | | 147.7 | | 150.8 | | 149.4 | | 149.7 |
| 3 | | 122 | | 122 | | 123.2 | | 123.1 | | 133.9 | | 134.6 |
| 4 | 4.57 | 76.6 | 4.58 | 77 | 3.88 | 42.1 | 3.88 | 42.1 | 4.53 | 70.2 | 4.53 | 70.6 |
| | 5.06 | | 5.07 | | 4.17 | | 4.18 | | | | | |
| 5 | 5.66 | 71.6 | 5.67 | 72 | 5.70 | 79.6 | 5.70 | 79.5 | 3.19 | 27.7 | 3.18 | 27.8 |
| 6 | | 140 | | 141 | | 139.8 | | 139.8 | | 138.7 | | 139.4 |
| 7 | | 130 | | 130 | | 130.8 | | 130.8 | | 136.2 | | 136.7 |
| 8 | 7.27 | 132 | 7.27 | 132 | 7.47 | 131.8 | 7.48 | 131.8 | 7.46 | 122.9 | 7.46 | 123.5 |
| 9 | | 142 | | 142 | | 144.2 | | 144.2 | | 133.9 | | 134.0 |
| 10 | 2.75 | 47 | 2.75 | 47 | 4.71 | 82.8 | 4.71 | 82.8 | | 211.0 | | 211.4 |
| 11 | | 41.1 | | 41 | | 45.3 | | 45.3 | | 45.8 | | 45.5 |
| 12 | | 170 | | 170 | | 169.6 | | 169.6 | 2.32 | 15.0 | 2.32 | 25.4 |
| 13 | 2.40 | 17.9 | 2.41 | 18 | 2.40 | 18.0 | 2.40 | 18.0 | 2.42 | 20.4 | 2.42 | 20.2 |
| 14 | 1.19 | 28.7 | 1.19 | 29 | 1.19 | 26.7 | 1.20 | 26.7 | 1.23 | 25.2 | 1.23 | 25.4 |
| 15 | 1.15 | 28.7 | 1.16 | 29 | 1.07 | 21.5 | 1.08 | 21.4 | 1.23 | 25.2 | 1.23 | 25.4 |

4-CH₃CO
4-CH₃CO
12-CH₃CO
12-CH₃CO

TABLE 19
NMR shift comparison between compounds isolated in
the current work to those previously published.

| Alcyopterosin G | | | | Bis(acetyl)alcyopterosin O | | | |
|---|---|---|---|---|---|---|---|
| This work | | Palermo¹⁶ | | This work | | Carbone¹⁷ | |
| δ_{H} | δ_{C} | δ_{H} | δ_{C} | δ_{H} | δ_{C} | ?_{H} | ?_{C} |
| 2.79 | 46.3 | 2.79 | 46.3 | 2.72 | 46.4 | 2.74 | 46.4 |
| | 141.5 | | 141.5 | | 142.4 | | 142.4 |
| | 135.0 | | 135.0 | | 135.5 | | 135.5 |
| 4.60 | 72.6 | 4.60 | 72.6 | 4.14 | 63.9 | 4.15 | 63.9 |
| 3.17 | 26.8 | 3.17 | 26.8 | 3.03 | 29.7 | 3.03 | 29.7 |
| | 130.6 | | 130.6 | | 132.8 | | 132.8 |
| | 135.5 | | 135.5 | | 130.3 | | 130.3 |
| 7.00 | 127.2 | | 127.2 | 7.01 | 127.7 | 7.01 | 127.7 |
| | 143.0 | | 143.0 | | 142.4 | | 142.4 |
| 2.70 | 47.7 | 2.70 | 47.7 | 2.69 | 47.6 | 2.69 | 47.6 |
| | 39.7 | | 39.7 | | 39.7 | | 39.7 |
| 4.69 | 60.4 | 4.69 | 60.4 | 5.15 | 62.1 | 5.15 | 62.1 |
| 2.34 | 19.9 | 2.34 | 19.9 | 2.35 | 20.0 | 2.35 | 20.0 |
| 1.15 | 29.0 | 1.15 | 29.0 | | 29.0 | | 29.0 |
| 1.15 | 29.0 | 1.15 | 29.0 | 1.14 | 29.0 | 1.14 | 29.0 |
| | | | | 2.06 | 21.0 | 2.06 | 21.0 |
| | | | | | 170.6 | | 170.6 |
| | | | | 2.07 | 21.0 | 2.07 | 21.0 |
| | | | | | 170.9 | | 170.9 |

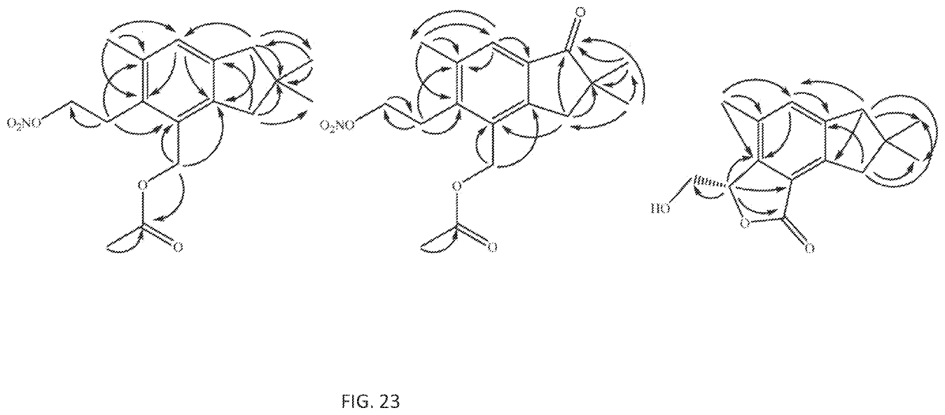

Alcyopterosin T (C1) displayed an HRESIMS [M+Na]⁺ at m/z 344.1460, which agrees well with C₁₇H₂₃NO₅Na (calcd m/z 344.1468), and sharp IR bands at 1640 and 1280 cm⁻¹ were consistent with the presence of a nitrate moiety. The ¹H NMR spectrum (Table 20) displayed nine well-resolved signals, two of which were coupled triplets while the other seven were singlets. The HSQC spectrum identified the nine protonated carbon signals, and the additional seven non-protonated carbon signals were evident from the HMBC spectrum. Six carbon shifts in the olefinic region could be cyclized into an aromatic ring based on HMBC correlations (FIG. 23) of the deshielded methyl group at δ_{H} 2.38 (C-13) to C-6 (δ_{C} 131.4), C-7 (ac 135.9) and C-8 (δ_{C} 128.0); H-8 (δ_{H} 7.06) to C-2 (δ_{C} 143.2), and C-6; H₂-10 (δ_{H} 2.73) to C-2 and C-8; H₂-1 (δ_{H} 2.79) to C-9 (δ_{C} 143.4); H₂-12 (δ_{H} 5.16) to C-2 and C-3 (δ_{C} 131.1); and H₂-5 (δ_{H} 3.15) to C-3 and C-7. Additional HMBC correlations between both H₂-1 and H₂-10 to C-11 (δ_{C}40.4) and C-14/15 (δ_{C} 29.7) established a fused five-membered ring on the aromatic ring.

TABLE 20
¹H and ¹³C NMR Data for Alcyopterosins T, U and V (C1-C3).

| | Alcyopterosin T (1) | | Alcyopterosin U (2) | | Alcyopterosin V (3) | |
|---|---|---|---|---|---|---|
| Position | δ_{C},¹ type | δ_{H},² mult. (J) | δ_{C},¹ type | δ_{H},² mult. (J) | δ_{C},¹ type | δ_{H},² mult. (J) |
| 1 | 47.1, CH₂ | 2.79, s | 211.4, C | | 44.8, CH₂ | 3.04, s |
| 2 | 143.2, C | | 135.4, C | | 141.1, C* | |
| 3 | 131.1, C | | 133.0, C | | 122.5, C | |
| 4 | 72.5, CH₂ | 4.57, t (7.9) | 28.6, CH₂ | 3.28, t (7.7) | 63.2, CH₂ | 4.25, dd (2.5, 12.6) |
| | | | | | | 3.81, dd (5.9, 12.6) |
| 5 | 27.7, CH₂ | 3.15, t (7.9) | 71.9, CH₂ | 4.60, t (7.6) | 82.2, CH | 5.55, dd (2.1, 5.0) |
| 6 | 131.4, C | | 138.2, C | | 130.0, C | |
| 7 | 135.9, C | | 142.2, C | | 142.4, C | |
| 8 | 128.0, CH | 7.06, s | 127.4, CH | 7.64, s | 131.8, CH | 7.24, s |
| 9 | 143.4, C | | 151.5, C | | 146.7, C* | |
| 10 | 48.5, CH₂ | 2.73, s | 42.2, CH₂ | 3.03, s | 47.0, CH₂ | 2.74, s |
| 11 | 40.4, C | | 46.3, C | | 40.9, C | |
| 12 | 62.5, CH₂ | 5.16, s | 61.0, CH₂ | 5.25, s | 170.8, C | |
| 13 | 20.7, CH₃ | 2.38, s | 21.0, CH₃ | 2.47, s | 18.0, CH₃ | 2.37, s |
| 14 | 29.7, CH₃ | 1.17, s | 26.2, CH₃ | 1.24, s | 28.8, CH₃ | 1.16, s |
| 15 | 29.7, CH₃ | 1.17, s | 26.2, CH₃ | 1.24, s | 28.8, CH₃ | 1.19, s |
| 1′ | 171.1, C | | 171.2, C | | | |
| 2′ | 21.7, CH₃ | 2.09, s | 21.8, CH₃ | 2.10, s | | |

Two additional substitutions were found on the aromatic ring of alcyopterosin T (C1). H₂-12, besides the HMBC correlations described above in the aromatic ring, further correlated (FIG. 23) to an ester-type carbonyl at δ_{C} 171.1 (C-1′), which could be elaborated into an acetate group based on the HMBC correlation of H₃-2′ (δ_{H} 2.09) to C-1′. And lastly, H₂-5 had both COSY correlations to H₂-4 (δ_{H} 4.57) and HMBC correlation to C-4 (δ_{C} 63.2), completing the ¹H and ¹³C assignments of C1. Missing from the molecular formula is N₀₃, and the sole open valence on C-4 establishes alcyopterosin T as the acylated alcyopterosin G [16].

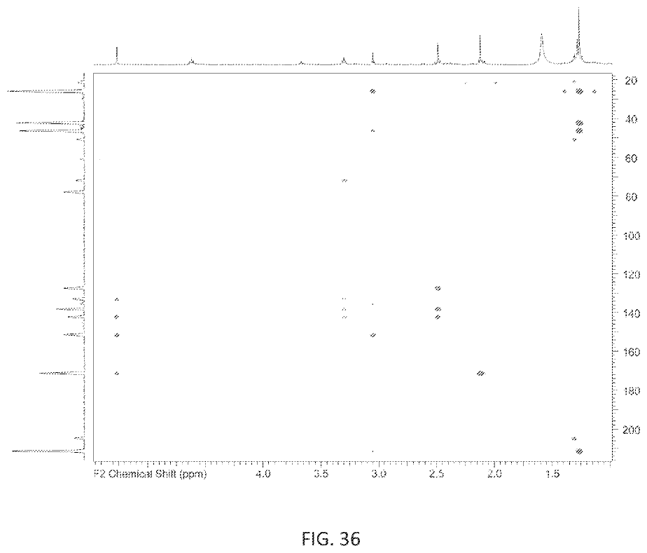

The spectral data for alcyopterosin U (C2) were very similar to those of C1 and again reminiscent of the alcyopterosin family of metabolites. The HRESIMS ([M+H]⁺: m/z 336.1429; calcd for C₁₇H₂₂NO₆: 336.1442) found that C2 has one additional oxygen and two protons fewer than C1. The IR spectrum displayed the same sharp bands at 1640 and 1280 cm⁻¹ supportive of the nitrate ester moiety, along with the absorptions at 1700 and 1750 cm⁻¹ typical of ketone and ester functions, respectively [16]. The most obvious difference between the ¹H NMR spectra of C1 and C2 was the absence of one methylene and the shift of the aromatic proton H-8, from δ_{H} 7.06 in C1 to 7.64 in C2. The HMBC spectrum demonstrated a correlation between the gem-dimethyl protons (H₃-14/15, δ_{H} 1.24) and a carbon signal at δ_{C} 211.4, reflecting a departure in C2 from the oxidation state of 1. Taken with the missing methylene group in C2, the ketone must be at C-1 or C-10. A methylene signal at δ_{H} 3.03 (H₂-1) also correlated in the HMBC spectrum to the ketone, as well as δ_{C} 151.5 and 135.4. Because H₂-12 (δ_{H} 5.25) also had an HMBC correlation to δ_{C} 151.5, but not to δ_{C} 135.4, then δ_{C} 151.5 must be C-2 and δ_{C} 135.4 must be C-9. An HMBC correlation between H₂-1 and C-3 secured the position of the carbonyl at C-10. Further ¹H and ¹³C shifts as well as HMBC correlations (FIG. 36) supported the remaining substitution on the aromatic ring of C2 mirroring that observed for C1.

The ¹H NMR spectrum of alcyopterosin V (C3) displayed a new pattern relative to those from 1 and 2, though certain resemblances remained. Lacking an acetoxy signal found in C1 and C2, the molecular formula of 3 was established as C₁₅H₁₈O₃ from the HRESIMS, in conjunction with the ¹³C NMR spectrum (Table 21), (C₁₅H₁₉O₃ [M+H]⁺: m/z 247.1328). The aromatic ring was established to be very much like that for C1: from the HMBC, a significantly deshielded/aromatic proton at δ_{H} 7.24 (H-8) correlated with δ_{C} 141.1 (C-2 or C-9) and 142.4 (C-6), the latter of which also had HMBC correlation from highly deshielded/aromatic methyl at δ_{H} 2.37 (H₃-13). The aromatic methyl showed further HMBC correlations to δ_{C} 130.0 (C-7) and 131.8 (C-8). With the observation of HMBC correlation of δ_{H} 5.55 (H-5) to C-6 and δ_{C} 122.5 (C-3), only C-2 and C-9 (δ_{C} 146.7 and 141.1) remained to secure as part of the aromatic ring. H-8, H₂-1 (δ_{H} 3.04), and H₂-10 (δ_{H} 2.74), the only hydrogen-bearing carbons near C-2 and C-9, are all 2 or 3 bonds apart and thus cannot assist in the assignment. Instead, we have assigned C-2 and C-9 based on their shift comparisons to similar carbons in C1 and C2, but we note that they may be interchanged.

Substitution on the aromatic ring of C3 was completed by considering the HMBC correlations of the remaining protons and carbons. H₂-1 and H₂-10 were noted above as correlated in the HMBC with both C-2 and C-9, locating them on the ring relative to already established H₃-13 and H-8; H₂-10 was distinguished from H₂-1 by HMBC correlation to C-8, disambiguating their relative positions. They also both correlated with C-11 (δ_{C} 40.9) and C14/15 (δ_{C} 28.8), completing the fused cyclopentane ring found on all the alcyopterosins. The final feature of alcyopterosin V was established by observation of the HMBC correlation of H-5 to both an oxymethylene (C-4, δ_{C} 63.2) and an ester-type carbonyl at δ_{C} 170.8 (C-12). As the protons of the oxymethylene (H-4a, δ_{H} 4.25; H-4b, δ_{H} 3.81) were COSY coupled to H-5, which was already affixed to the aromatic ring at C-6 as described above, the ester carbonyl must be located at C-3, completing a lactone ring. Insufficient material for optical spectra prevented comparison of the configuration of C-5 in C3 and alcyopterosin E (C4), but C3 represents the nitrate ester hydrolysis product of C4, due to which we suggest the two will share a common configuration. Additional support for the assigned configuration comes from an analysis of the coupling constants for the chiral proton H-5 of C3, which match those of C4 in magnitude (3: ³J₄ₐ₋₅=2.5 Hz, ³J_{4b-5}=6.1 Hz; 4: ³J₄ₐ₋₅=2.3 Hz, ³J_{4b-5}=6.6 Hz).

Further work was done to bring forward additional alcyopterosins, and a subsequent extraction was conducted and similarly fractionated. Alcyosterone (C5) eluted late in the silica gradient (hexanes to ethyl acetate), suggesting a moderately polar metabolite. Upon analysis, it was determined to have the molecular formula C₃₃H₅₀O₈ based on HRESIMS data that was corroborated by proton and carbon counts from their NMR spectra (Table 21). From the HRESIMS, the [M+H]⁺ was observed at m/z 575.3555, and [M−HOAc]⁺ was observed at m/z 515.3364. Analysis of the ¹³C NMR spectrum supported the 33 carbons accounted for by the MS and further indicated a ketone (C-1, δ_{C} 203.9), three ester-type carbons (C-1′, δ_{C} 169.4; C-3′, δ_{C} 170.4; C-5′, δ_{C} 169.9), two olefinic carbons (C-2, δ_{C} 128.4; C-3, δ_{C} 142.5), and four carbon signals in the oxygen-bearing region (C-6, δ_{C} 69.7; C-11, δ_{C} 70.4; C-15, δ_{C} 70.5; C-16, δ_{C} 73.0). The HSQC established the two olefinic carbons and all four of the oxygen-bearing carbons as methines and further indicated five aliphatic methines, six aliphatic methylenes, and eight methyl carbons. The ¹H NMR spectrum provided few additional insights into this overview of alcyosterone other than to suggest that three of the methyl carbons were associated with acetate esters, based on their chemical shifts (H₃-2′, δ_{H} 1.93; H₃-4′, δ_{H} 2.06; H₃-6′, δ_{H} 2.02) and HMBC correlation to their respective ester carbonyl.

TABLE 21
¹H and ¹³C NMR Spectroscopic Data for Alcyosterone (C5).

| Position | δ_{C}¹, type | δ_{H}² integ., mult., J |
|---|---|---|
| 1 | 203.9, C | |
| 2 | 128.4, CH | 5.83, 1H, dd, 2.2, 9.9 |
| 3 | 142.5, CH | 6.58, 1H, ddd, 2.1, 4.8, 9.6 |
| 4 | 28.4, CH₂ | 2.79, 1H, dddd, 0.7, 2.4, 11.4, 19.8 |
| | | 2.11, 1H, ddd, 0.8, 4.8, 19.5 |
| 5 | 46.6, CH | 1.86, 1H, ddd, 0.7, 2.9, 10.8 |
| 6 | 69.7, CH | 3.87, 1H, q, 2.4 |
| 7 | 36.8, CH₂ | 1.74, 1H, ov³ |
| | | 1.21, 1H, ov |
| 8 | 24.9, CH | 2.23, 1H, ov |
| 9 | 47.8, CH | 2.07, 1H, ov |
| 10 | 47.7, C | |
| 11 | 70.4*, C | 5.02, 1H, dt, 3.9, 11 |
| 1 | 46.7, CH₂ | 2.20, 1H, ov |
| 13 | 43.7,CH | |
| 14 | 56.6, CH | 1.31, 1H, dd, 5.8, 11.2 |
| 15 | 70.5*, CH | 5.34, 1H, dd, 6.3, 6.6 |
| 16 | 73.0, CH | 5.51, 1H, dd, 6.9, 7.0 |
| 17 | 59.9, CH | 1.34, 1H, ov |
| 18 | 15.8, CH₃ | 1.22, 3H, s |
| 19 | 13.2, CH₃ | 1.28, 3H, s |
| 20 | 30.0, CH | 1.76, 1H, ov |
| 21 | 18.2, CH₃ | 0.95, 3H, d, 6.6 |
| 22 | 35.6, CH₂ | 1.20, 1H, ov |
| | | 0.90, 1H, ov |
| 23 | 24.4, CH₂ | 1.36, 1H, ov |
| | | 1.11, 1H, ov |
| 24 | 39.1, CH₂ | 1.09, 1H, ov |
| | | 1.05, 1H, d, 6.6 |
| 25 | 27.9, CH | 1.48, 1H, ov |
| 26 | 22.6 CH₃ | 0.85, 3H, d, 6.5 |
| 27 | 22.4 CH₃ | 0.85, 3H, d, 6.5 |
| | 170.4, C | |
| 2′ | 21.5, CH₃ | 1.93, 3H, s |
| 3′ | 169.9, C | |
| 4′ | 20.7, CH₃ | 2.06, 3H, s |
| 5′ | 169.4, C | |
| 6′ | 20.5, CH₃ | 2.02, 3H, s |

Extending the cyclohexenone, H₂-4 further coupled in the HMBC spectrum to an oxymethine, C-6, and displayed a COSY correlation to H-5 (δ_{H} 1.86), the latter of which has an HMBC correlation with C-9 (δ_{C} 47.8). H-6 (δ_{H} 3.87) shows a COSY correlation to H₂-7 (a: δ_{H} 1.74; b: δ_{H} 1.21), and HMBC correlation with quaternary C-10 and the methine C-8 (δ_{C} 24.9). H-8 (δ_{H} 2.23) correlates in the HMBC with C-10, establishing a decalin ring system with the new cyclohexane ring fused to the cyclohexenone ring. A pendant methyl group (H₃-19, δ_{H} 1.28) with HMBC correlations to C-1 and C-10 must be placed at the ring junction. H-8 further correlates in the HMBC with C-14 (δ_{C} 56.6) and C-11. COSY correlations between H-9 (δ_{H} 2.07) and H-11 (δ_{H} 5.02), then H-11 and H₂-12 (a: δ_{H} 2.20; b: δ_{H} 1.48) support an extended branch from the decalin system that, taken with HMBC correlations for H₂-12 to C-11, C-18 (δ_{C} 15.8)m and C-13 (δ_{C} 43.7), and H₃-18 (δ_{H} 1.22) to C-13 and C-14 (δ_{C} 56.6), establishes a third ring fused to the previously established decalin. A fourth ring, the five-membered ring of a steroid ring system, was established by observation of a COSY correlation between H-14 (δ_{H} 1.31) and H-15 (δ_{H} 5.34), between H-15 and H-16 (δ_{H} 5.51), and between H-16 and H-17 (δ_{H} 1.34), all of which were HMBC correlated with C-13.

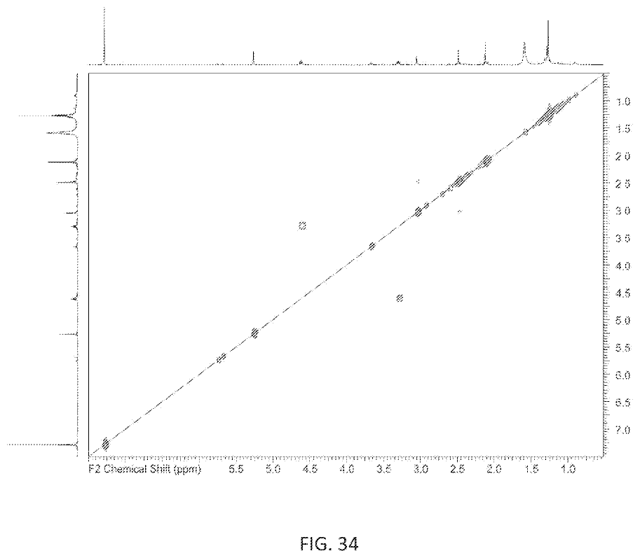

Left to assign were the steroid side chain and the acetate groups. The two ends of the steroid side chain were readily determined by HMBC correlations among the protons and carbons of positions 17, 20, 21, and 22, as well as 24, 25, and 26/27. Very weak correlations could be discerned between C-23 (δ_{C} 24.4) and H-22b (δ_{H} 0.90) and H-20 (δ_{H} 1.76), as well as H-23a (δ_{H} 1.36) and C-24 (δ_{C} 39.1), but overlapping and otherwise weak signals made assignments of C-23 to the rest of the well-established side chain challenging. The positions of the acetate groups were readily established by HMBC correlation of the oxymethine protons to the attached ester carbonyl; similarly, the acetate methyl groups could be positioned on their respective carbonyls FIG. 34).

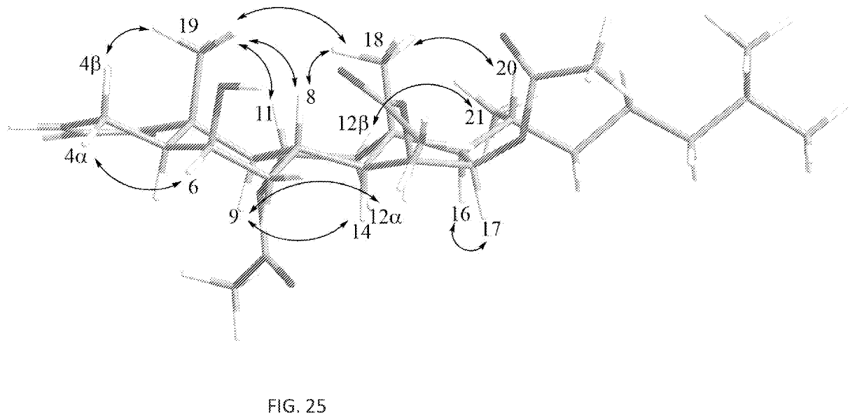

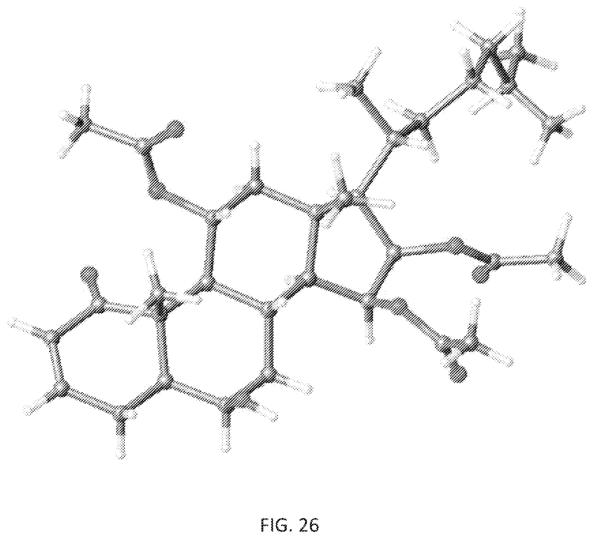

The stereochemical features of alcyosterone (C5) were studied by ROESY and X-ray diffraction (XRD) analysis. Many of the relative relationships could be discerned in the ROESY spectrum (FIG. 25), including methyl group H₃-19 (δ_{H} 1.28), H-4β, H-8, and H-11 co-locating on the same face of the ring system and defining the A/B rings as a trans-decalin. Additional relationships were evident between H₃-18, H-20, and H-8; H-12α and H₃-21; H-9 and H-14; H-9 and H-12α; H-16 and H-17; H-15 and H-7β; and H-6 and H-4β (see FIG. 25). These relationships were confirmed by XRD, which also provided the absolute stereochemistry (FIG. 26).

Alcyopterosins are known to be mildly cytotoxic toward human tumor cell lines [16,19] but little attention has been focused on their infectious disease (ID) activity. Metabolites from Alcyonium sp. indet. isolated in this study in sufficient quantity were therefore screened in three ID assays. Alcyopterosins V (C3), E (C4), and alcyosterone (C5) were inactive against the ESKAPE panel of bacterial pathogens, but both C3 and C4 demonstrated potent activity against Clostridium difficile, a difficult-to-treat intestinal bacterium which afflicts up to half a million people annually and caused 30,000 deaths in 2015 [20]. Alcyopterosin E (MIC 6.9 PM) was slightly more active against C. difficile than alcyopterosin V (MIC 8.1 μM). Cytotoxicity against host cell lines HEK293T and HepG2 also found C4 less toxic (CC₅₀ 570 and 331 μM, respectively) than C3 (CC₅₀ 220 and 288 μM, respectively). Vancomycin, as a control, displays an MIC of 0.34 μM against C. difficile and was non-toxic to the host cells at the same concentrations alcyopterosins were assayed.

Alcyopterosin C, E (C4), L, 4,12-bis(acetyl)alcyopterosin O, V (C3), and alcyosterone (C5) were screened against Leishmania donovani and found with roughly equal, single digit M, activity [21]. Leishmania, the disease caused by L. donovani, is often disfiguring and can lead to death if not properly treated, though current treatment regimens can be expensive and toxic, and are considered ineffective [22]. The highest potency was displayed by 4,12-Bis(acetyl)alcyopterosin O (IC₅₀ 1.2 μM), though alcyosterone (IC₅₀ 1.5 μM), alcyopterosin L (IC₅₀ 2.4 μM), and alcyopterosin E (IC₅₀3.1 μM) were largely indistinguishable. Alcyopterosin V (IC₅₀7.0 μM) and alcyopterosin C (IC₅₀ 13 μM) were only slightly less potent than the control, miltefosine (IC₅₀6.2 μM). Only C3 and C4 were available in sufficient quantity to assay against the Leishmania host cell line, J774.A1 macrophages, which showed alcyopterosin E, though low in toxicity, was twice as toxic (IC₅₀ 62 μM) as alcyopterosin V (IC₅₀ 110 μM) to the mammalian cells.

### Materials and Methods

Optical rotations were measured on a Rudolph Research Analytical AUTOPOL IV digital polarimeter at 589 nm. UV absorptions were acquired with an Agilent Cary 60 UV-vis spectrophotometer. IR spectra were recorded with an Agilent Cary FTIR 630 spectrometer and PerkinElmer Spectrum Two equipped with a UATR (single reflection diamond) sample introduction system. NMR spectra were recorded on Varian Direct Drive 500 MHz and Varian Inova 500 MHz spectrometers. Chemical shifts are reported with the use of the residual CDCl₃ signals (δ_{H} 7.27 ppm; δ_{C} 77.0 ppm) as internal standards for ¹H and ¹³C NMR spectra, respectively. COSY, HSQC, HMBC, and ROESY experiments corroborated the ¹H and ¹³C NMR assignments. Analytical LC/MS with a Phenomenex Kinetex C18 column (50×2.1 mm, 2.6 m) on an Agilent 6230 LC/TOF-MS with electrospray ionization detection provided the high-resolution masses. Semi-preparative and analytical HPLC separations were performed on a Shimadzu LC-20 AT system equipped with an ultraviolet (UV) detector using a Luna silica column (5 μm, 250×10 mm), and a YMC C-18 column (10 m, 150×4 mm). MPLC was performed on a Teledyne Isco CombiFlash Rf 200i equipped with an evaporative light-scattering detector (ELSD) and a multiwavelength UV detector using a RediSep Rf silica 80 g flash column, and silica gel 230-400 mesh was used to load samples.

The soft coral was collected via trawling on the R/V Nathaniel B. Palmer vessel during the austral autumn in late April 2013. The specimens were collected between 126 and 130 m depth, frozen immediately upon collection, and maintained at −80° C. until extraction. The tissue of the frozen specimens was subsampled and preserved in 96% ethanol. Subsequent extraction was performed using a DNeasy blood and tissue kit (Qiagen) following manufacturer's protocols. Using primers ND42599F/mut3458R [23,24], a piece of the mitochondrial genome was amplified (msh1, a homolog of mutS). Cycling conditions included an initial 5× cycles at 45° C. annealing, followed by 39× cycles at 58° C. Amplicons were sent to the Australian Genome Research Facility, Perth for purification and Sangar sequencing. The resulting bi-directional sequence was assembled and edited, primers removed, deposited in GenBank (Accession No. OP429120), and aligned with other soft coral sequences from GenBank. A Maximum-Likelihood analysis using IQ-tree [25], implementing the evolutionary model VM+F+G4 selected with ModelFinder [26], was carried out. The nodes were tested with 1000 ultrafast bootstrap replicates.

The frozen soft coral was freeze-dried, and 420 g of dry weight material was extracted using a 1:1 ratio of dichloromethane/methanol, three times over 3 days. The extract was dried, and the yielded 25.0 g was resolubilized in ethyl acetate and partitioned against H₂O. The concentrated EtOAc partition fraction (11.4 g) was resuspended in EtOAc and dried onto silica gel for fractionation by MPLC on a Teledyne CombiFlash fitted with UV and ELS detection. Fractions A through I eluted from MPLC using ethyl acetate/n-hexanes (0:100) to ethyl acetate/n-hexanes (100:0) over 25 min. Fractions D through H displayed NMR signature signals of marine illudalane compounds, in particular the aromatic singlet (H-8) and a midfield oxymethylene (H₂-4), and were selected for purification using normal-phase and reversed-phase HPLC with UV detection. Semi-preparative NP HPLC using n-hexane to EtOAc/n-hexanes (1:1) over 25 min gradient, yielded the known alcyopterosins C (C6), G (C7), and 4,12-bis(acetyl)alcyopterosin O (C8) from MPLC fraction F. Alcyopterosin L (C9) and newly isolated as natural product alcyopterosin V (C3) (4.0 mg) came from MPLC fraction H. Alcyopterosin E (C4) was derived from fraction G. New alcyopterosins T (C1) (0.5 mg) and U (C2) (0.5 mg) came from fraction E, along with 4,12-bis(acetyl)alcyopterosin O (1.6 mg) and alcyopterosins C (2.0 mg), E (7.5 mg), G (0.6 mg), and L (1.4 mg).

Soxhlet extraction of an additional specimen in dichloromethane followed by a similar chromatographic profile described above resulted in seven fractions. Further purification of fraction E, via normal phase HPLC with a hexane-ethyl acetate (1:1) gradient, followed by reversed-phase HPLC using a water-acetonitrile (70% to 100%) gradient, led to alcyosterone (5) (1.2 mg).

Alcyopterosin T (C1): colorless oil; UV (CH₂Cl₂) λₘₐₓ (log ε): 225 (1.52), 245 (1.45), 340 (1.24) nm; IR vₘₐₓ: 3000, 2900, 2850, 1720, 1640, 1600, 1280 cm⁻¹; for ¹H and ¹³C NMR data see 20; HRESIMS [M+Na]⁺: m/z 344.1460 (calcd for C₁₇H₂₃NO₅Na, m/z 344.1468).

Alcyopterosin U (C2): colorless oil; UV (CH₂Cl₂) λₘₐₓ (log ε): 225 (1.76), 230 (1.59), 250 (1.55), 264 (1.54), 305 (1.52), 330 (1.47), 365 (1.44) nm; IR vₘₐₓ: 3000, 2900, 2850, 1750, 1700, 1640, 1600, 1280 cm⁻¹; for ¹H and ¹³C NMR data see table 20; HRESIMS [M+H]⁺: m/z 336.1429 (calcd for C₁₇H₂₂NO₆, m/z 336.1442).

Alcyopterosin V (C3): for ¹H and ¹³C NMR data see table 20. HRESIMS [M+H]⁺: m/z 247.1328 (Calcd for C₁₅H₁₉O₃, 247.1329).

Alcyosterone (C5): translucent solid; [α]^{24.6}₃₆₅ −125° (c 2×10⁻³ g/mL, ACN); UV (ACN) λₘₐₓ (log ε): 215 (2.60), 235 (2.68) nm; IR υₘₐₓ: 1250, 1690, 1700, 1750, 2850, 2900, 2950 cm⁻¹; for ¹H and ¹³C NMR data see table 21; HRESIMS [M+H]⁺: m/z 575.3555 (calcd for C₃₃H₅₀O₈H, n/z 575.3578); [M−OAc]⁺ m/z 515.3364 (calcd for C₃₁H₄₇O₆, n/z 515.3367).

The Leishmania donovani infected macrophage assay and cytotoxicity screen were conducted as previously described [27].

The screening against C. difficile was performed in two steps. In step 1, overnight culture of a hyper-virulent clinical strain C. difficile UK6 was inoculated into a fresh BHIS medium at a volume ratio of 1:1000. After pre-incubation at 37° C. under an anaerobic atmosphere for 2 h, the bacterial culture was divided into a sterile 96-well plate and each well contained 192 μL of bacterial culture. Then, 8 μL of each extract was added to each well of the plate, mixed thoroughly, and incubated at 37° C. in an anaerobic chamber for 48 h. Control groups of 200 μL of BHIS medium only, 200 μL of bacterial culture only, and 192 μL of bacterial culture in 8 μL of DMSO were also included in separate columns within each plate. Extracts that displayed initial antibacterial activity were further evaluated for their minimum inhibitory concentration (MIC) against C. difficile. Serial dilutions of each extract (400 g/mL, 200 μg/mL, 100 μg/mL, 50 μg/mL, 20 μg/mL, 10 μg/mL, 5 μg/mL, and 2 μg/mL) were prepared in a fresh BHIS medium. Then, 100 μL of each extract dilution was added to 100 μL of bacterial culture (pretreated as described), mixed well, and incubated at 37° C. in an anaerobic chamber for 48 h. Control groups including wells containing fresh medium only and bacterial culture only were also included as described. Activity was determined as +/−(clear or turbid (OD₆₀₀) culture). The MICs of the three recommended antibiotics metronidazole, vancomycin, and fidaxomicin against C. difficile UK6 were also determined using broth microdilution methodology.

The cytotoxicity of the metabolites to human liver cells and kidney cells was determined using an MTT based-In Vitro Toxicology Assay Kit (Sigma-Aldrich, St. Louis, MO, USA) following the manufacturing instructions. The human kidney HEK293T cells and the human liver HEPGZ cells were used for the evaluation in this study. Both cell samples were maintained and suspended in Dulbecco's Modified Eagle Medium (DMEM with 4.5 g/L glucose, L-glutamine and sodium pyruvate, Corning, Manassas, VA, USA) containing 10% fetal bovine serum (Thermo Scientific) and 1% penicillin/streptomycin at 37° C. under 5% CO₂ atmosphere. The cells were plated on a 96-well plate with approximately 5×10³-1×10⁴ cells in each well, and incubated at 37° C. overnight. After that, each of the selected extracts from the antimicrobial susceptibility test was added to the wells and incubated with the cells at a series of 2-fold diluted concentrations ranging from 128 μg/mL to 0.125 μg/mL. Following a 24 h of incubation, 10 μL of 1-(4,5-dimethylthiazol-2-yl)-3,5-diphenylformazan (MTT) stock solution (5 mg/mL) was added to each well of the cells, mixed well, and incubated at 37° C. for another 4 h. After that, the liquid in each well of the plate was removed carefully and thoroughly, then the cells in the wells were treated with 100 μL of DMSO, and incubated at 37° C. for 15 m. Optical density (OD) values were measured at a wavelength of 540 nm (OD₅₄₀) using a microplate reader (Synergy HTX; Bio Tek Instruments, Inc. Winooski VT). Cells treated with vancomycin, a common option for treating CDI in clinical settings, were also included in the MTT tests as a control. Cell survival and the IC₅₀ were calculated according to the method used in a previous publication [26]: Survival of cells (%)=Drug-treated group OD₅₄₀/control group OD₅₄₀×100. The IC₅₀ value was calculated as follows: lgIC₅₀=Xₘ−I [P−(3−Pₘ−Pₙ)/4], where Xₘ was the log maximum dose, I was the log (maximum dose/adjacent dose), P was the sum of the positive response rate, Pₘ was the maximum positive response rate, and Pₙ was the minimum positive response rate.

XRD methodology was conducted as we have previously done [28]. Data and refinement conditions are shown in table 22. X-ray crystallographic data was deposited with the Cambridge Crystallographic Data Center (Deposition Number 2205919). The mutS gene sequence was deposited with Genbank (accession number OP429120)

TABLE 22
Crystal data and structure refinement for alcyosterone (C5).

| Identification code | ACDL_21 |
|---|---|
| Empirical formula | C33H50O8 |
| Formula weight | 574.73 |
| Temperature/K | 100.0 |
| Crystal system | orthorhombic |
| Space group | P2₁2₁2₁ |
| a/Å | 6.9203(5) |
| b/Å | 14.3360(10) |
| c/Å | 32.173(2) |
| α/° | 90 |
| β/° | 90 |
| γ/° | 90 |
| Volume/Å³ | 3191.8(4) |
| Z | 4 |
| ρcalcg/cm³ | 1.196 |
| μ/mm−1 | 0.681 |
| F(000) | 1248.0 |
| Crystal size/mm3 | 0.042 × 0.036 × 0.018 |
| Radiation | CuKα (λ = 1.54178) |
| 2Θ range for data collection/° | 5.494 to 137.154 |
| Index ranges | −7 ≤ h ≤ 8, −16 ≤ k ≤ 17, −38 ≤ l ≤ 38 |
| Independent reflections | 5867 [Rint = 0.1453, Rsigma = 0.0776] |
| Data/restraints/parameters | 5867/258/437 |
| Goodness-of-fit on F₂ | 1.026 |
| Final R indexes [I >= 2σ (I)] | R₁ = 0.0659, wR₂ = 0.1389 |
| Final R indexes [all data] | R₁ = 0.1009, wR₂ = 0.1577 |
| Largest diff. peak/hole/eÅ⁻³ | 0.40/−0.26 |
| Flack parameter | −0.10(19) |

### Example 8

New Alcyopterosins and Steroids Isolated from an Undescribed Antarctic Coral.

Marine invertebrates from Antarctica have been investigated for their potential natural product chemistry. Often sessile, these organisms must develop chemical protective mechanisms to survive and defend themselves against predators. The biodiversity of these organisms is of particular interest due to the extremely low temperatures and the circumpolar current around the Antarctic continent serving as an ecological isolating shield. The chemodiversity that emanates from these organisms can be a significant source of novel chemistry to be further developed into new drugs.

The chemical investigation of an undescribed Antarctic coral has led to the isolation of two different kinds of bioactive compounds. After lyophilization of the organism, two different extractions were performed: the first one used a methylene chloride: methanol (1:1) solvent mixture, and the second one used methylene chloride only as solvent in a Soxhlet extraction. After a partition followed by normal phase Medium Pressure Liquid Chromatography, stages of normal phase and reverse phase High Performance Liquid Chromatography purifications were performed and revealed two kinds of new bioactive compounds: new acetylated sesquiterpenoids with alcyopterosin scaffolds from the first process and new acetylated steroids from the second. One and two-dimensional nuclear magnetic resonance, mass spectrometry, X-ray crystallography, and circular dichroism were the methods performed to elucidate and confirm the structures. Furthermore, biological testing against Leishmania sp. and ESKAPE pathogens, Zika virus, Clostridium difficile, and HeLa cancer cells were performed to extend the scope of drug discovery potential.

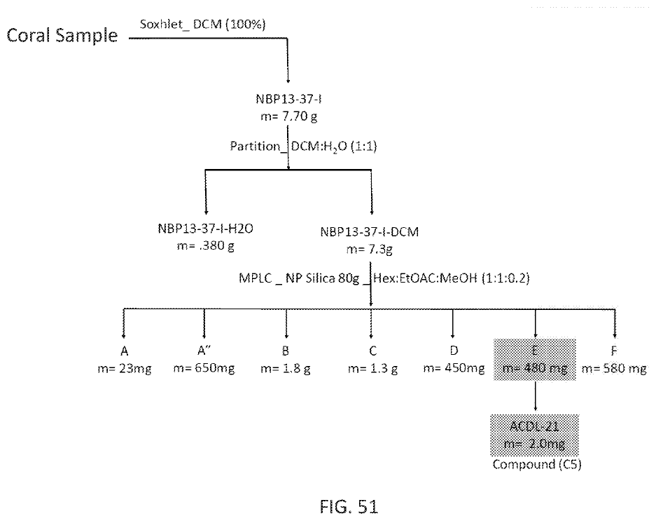

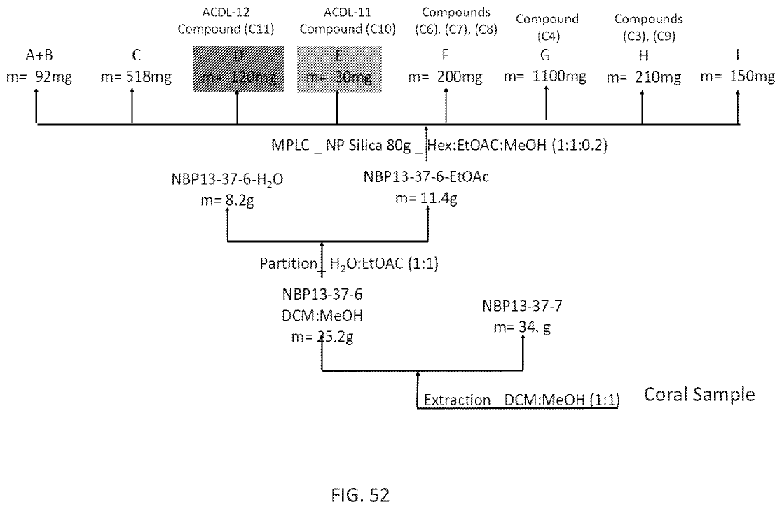

FIGS. 51 and 52 illustrate the extraction method for several of the compounds disclosed in this application. For example, in FIG. 51, compounds are extracted from the coral sample via a Soxhlet extract protocol, leading to the isolation of several fractions (e.g., Fractions A-F). From fraction F, 2.0 mg of compound (C5) was isolated. In another example, in FIG. 52, compounds are extracted from the coral sample via a dichloromethane/methanol (1:1) extraction protocol, leading to the isolation of eight fractions, from which compounds (C3)-(C4) and (C6-C9) were isolated. Further, new compounds ACDL-11 (Compound (10), and ACDL-12 (Compound (C11) were isolated.

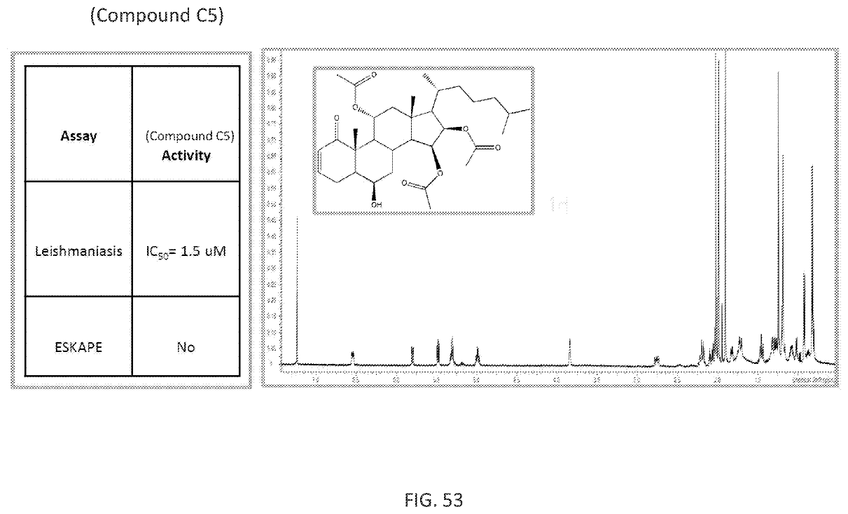

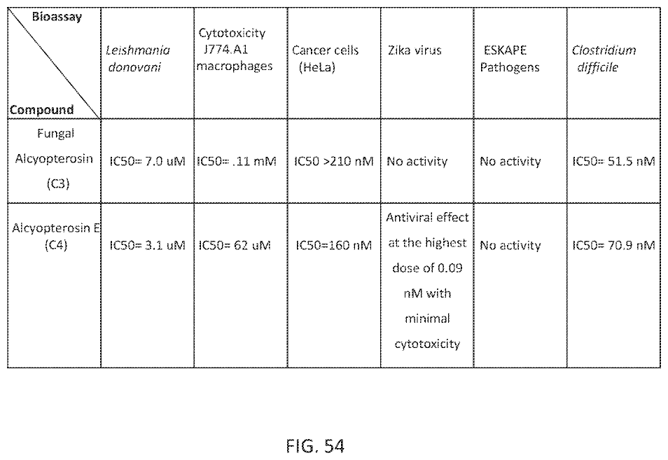

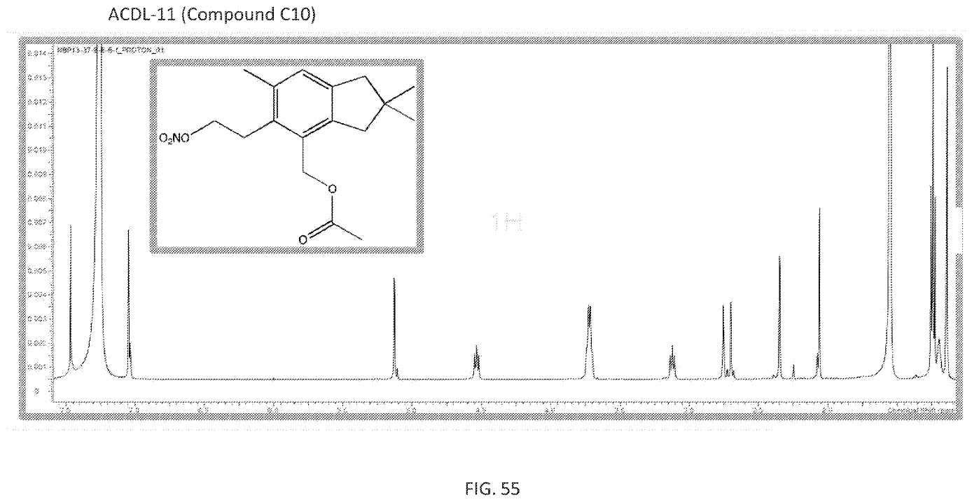

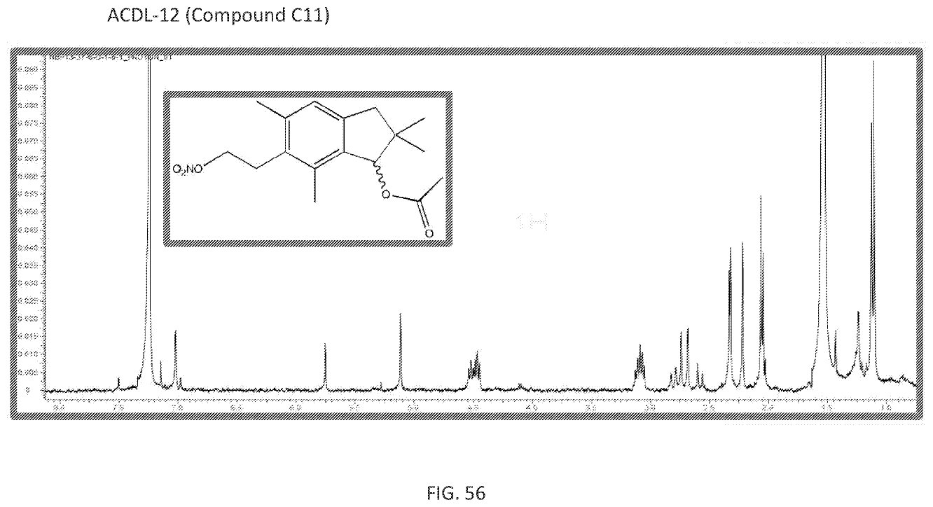

Several of the isolated compounds were identified by NMR analysis and/or tested for antimicrobial activity. For example, FIG. 53 illustrates an ¹H NMR spectrum of compound (C5), the elucidated structure of compound (C5), as well as the assayed ability of compound C5 to inhibit Leishmaniasis donovani and ESKAPE pathogens. ESKAPE is an acronym for the group of bacteria, encompassing both Gram-positive and Gram-negative species, made up of Enterococcus faecium, Staphylococcus aureus, Klebsiella pneumoniae, Acinetobacter baumannii, Pseudomonas aeruginosa, and Enterobacter species. FIG. 54 is a table illustrating the inhibition of various pathogens by compounds (C3) and (C4). FIGS. 55-56 illustrate ¹H NMR spectrums and elucidated structures of compounds (C10) and (C11), respectively.

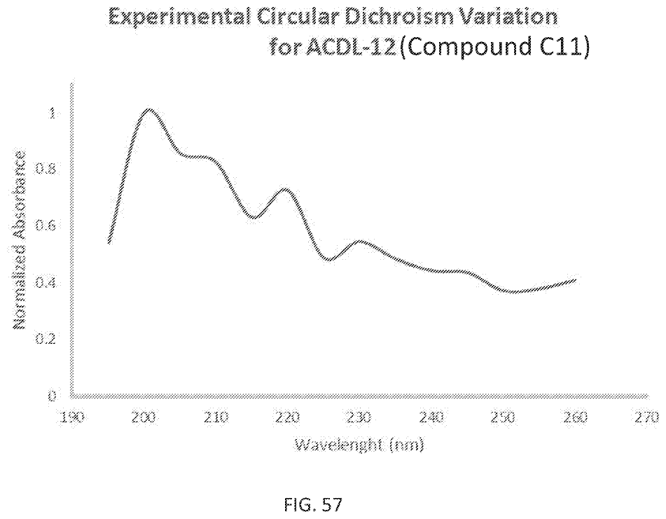

FIG. 57 is a graph illustrating the experimental circular dichroism variation for compound (C11). Here, the absolute stereochemistry of the carbon bearing the acetate moiety is assessed by comparison of experimental and calculated circular dichroism values. Further calculation of values of R and S enantiomers, and the calculated value matching the sign of the experimental trend will lead to the absolute stereochemistry of compound (C11).

### REFERENCES

1. Tsao F., Morgan L. E. Corals that live on mountaintops. J. Mar. Ed. 2005; 21:9-11.
2. Morgan L. E. What are deep-sea corals? J. Mar. Ed. 2005; 21:2-4.
3. Moore J. M., Carvajal J. I., Rouse G. W., Wilson N. G. The Antarctic Circumpolar Current isolates and connects: Structured circumpolarity in the sea star Glabraster antarctica. Ecol. Evol. 2018; 8:10621-10633. doi: 10.1002/ece3.4551.
4. Duenas L. F., Tracey D. M., Crawford A. J., Wilke T., Alderslade P., Sanchez J. A. The Antarctic Circumpolar Current as a diversification trigger for deep-sea octocorals. BMC Evol. Biol. 2016; 16:2. doi: 10.1186/s12862-015-0574-z.
5. von Salm J. L., Wilson N. G., Vesely B. A., Kyle D. E., Cuce J., Baker B. J. Shagenes A and B, new tricyclic sesquiterpenes produced by an undescribed Antarctic octocoral. Org. Lett. 2014; 16:2630-2633. doi: 10.1021/ol500792x.
6. Thomas S. A. L., von Salm J. L., Clark S., Nemani P., Ferlita S., Wilson N. G., Baker B. J. Keikipukalides, furanocembranoid aldehydes from the deep sea Antarctic coral Plumerella delicatissima. J. Nat. Prod. 2018; 81:117-123. doi: 10.1021/acs.jnatprod.7b00732.
7. Núñez-Ponz L., Shilling A. J., Verde C., Baker B. J., Giordano D. Marine terpenoids from polar latitudes and their potential applications in biotechnology. Mar. Drugs. 2020; 18:401. doi: 10.3390/md18080401.
8. Soldatou S., Baker B. J. Cold-water marine natural products, 2006 to 2016. Nat. Prod. Rep. 2017; 34:585-626. doi: 10.1039/C6NP00127K.
9. von Salm J. L., Schoenrock K. M., McClintock J. B., Amsler C. D., Baker B. J. The status of marine chemical ecology in Antarctica: Form and function of unique high-latitude chemistry. In: Puglisi M. P., Becerr M. A., editors. Chemical Ecology: The Ecological Impacts of Marine Natural Products. CRC Press; Boca Raton, FL, USA: 2018.
10. Rocha J., Peixe L., Gomes N. C. M., Calado R. Cnidarians as a source of new marine bioactive compounds—An overview of the last decade and future steps for bioprospecting. Mar. Drugs. 2011; 9:1860-1886. doi: 10.3390/md9101860.
11. Leal M. C., Puga J., Serodio J., Gomes N. C., Calado R. Trends in the discovery of new marine natural products from invertebrates over the last two decades—where and what are we bioprospecting?PLoS ONE. 2012; 7:e30580. doi: 10.1371/journal.pone.0030580.
12. Paul V. J., Puglisi M. P. Chemical mediation of interactions among marine organisms. Nat. Prod. Rep. 2004; 21:189-209. doi: 10.1039/b302334f.
13. Rodriguez Brasco M. F., Seldes A. M., Palermo J. A. Paesslerins A and B: Novel tricyclic sesquiterpenoids from the soft coral Alcyonium paessleri. Org. Lett. 2001; 3:1415-1417. doi: 10.1021/o1006684x.
14. Iken K. B., Baker B. J. Ainigmaptilones, sesquiterpenes from the Antarctic gorgonian coral Ainigmaptilon antarcticus. J. Nat. Prod 2003; 66:888-890. doi: 10.1021/np030051k.
15. McMorris T. C., Anchel M. The structures of the basidiomycete metabolites Illudin S and Illudin M. J Am. Chem. Soc. 1963; 85:831-832. doi: 10.1021/ja00889a052.
16. Palermo J. A., Rodriguez Brasco M. F., Spagnuolo C., Seldes A. M. Illudalane sesquiterpenoids from the soft coral Alcyonium paessleri: The first natural nitrate esters. J. Org. Chem. 2000; 65:4482-4486. doi: 10.1021/jo991740x.
17. Carbone M., Ninez-Pons L., Castelluccio F., Avila C., Gavagnin M. Illudalane sesquiterpenoids of the alcyopterosin series from the Antarctic marine soft coral Alcyonium grandis. J. Nat. Prod. 2009; 72:1357-1360. doi: 10.1021/np900162t.
18. Orfali R., Perveen S., Khan M. F., Ahmed A. F., Wadaan M. A., Al-Taweel A. M., Alqahtani A. S., Nasr F. A., Tabassum S., Luciano P., et al. Antiproliferative illudalane sesquiterpenes from the marine sediment ascomycete Aspergillus oryzae. Mar. Drugs. 2021; 19:333. doi: 10.3390/md19060333.
19. Finkielsztein L. M., Bruno A. M., Renou S. G., Moltrasio Iglesias G. Y. Design, synthesis, and biological evaluation of alcyopterosin A and illudalane derivatives as anticancer agents. Bioorg. Med. Chem. 2006; 14:1863-1870. doi: 10.1016/j.bmc.2005.10.033.
20. Leffler D. A., Lamont J. T. Clostridium difficile infection. N. Eng. J. Med. 2015; 373:287-288. doi: 10.1056/NEJMra1403772.
21. Baker B. J., Wilson N. G., Kyle D. E., Limon A.-C. Leishmania inhibitors. 10898460-B1. U.S. Patent. 2021 January 26;
22. Salari S., Bamorovat M., Sharifi I., Almani P. G. N. Global distribution of treatment resistance gene markers for leishmaniasis. J. Clin. Lab. Anal. 2022; 36:e24599. doi: 10.1002/jcla.24599.
23. France S. C., Hoover L. L. Analysis of variation in mitochondrial DNA sequences (ND3, ND4L, MSH) among Octocorallia (=Alcyonaria)(Cnidaria: Anthozoa) Bull. Biol. Soc. Wash. 2001; 10:110-118.
24. France S. C., Hoover L. L. DNA sequences of the mitochondrial COI gene have low levels of divergence among deep-sea octocorals (Cnidaria: Anthozoa) Hydrobiologia. 2002; 471:149-155. doi: 10.1023/A:1016517724749.
25. Trifinopoulos J., Nguyen L.-T., von Haeseler A., Minh B. Q. W-IQ-TREE: A fast online phylogenetic tool for maximum likelihood analysis. Nucleic Acids Res. 2016; 44:W232-W235. doi: 10.1093/nar/gkw256.
26. Kalyaanamoorthy S., Minh B. Q., Wong T. K. F., von Haeseler A., Jermiin L. S. ModelFinder: Fast model selection for accurate phylogenetic estimates. Nat. Methods. 2017; 14:587-589. doi: 10.1038/nmeth.4285.
27. Shilling A. J., Witowski C. G., Maschek J. A., Azhari A., Vesely B., Kyle D. E., Amsler C. D., McClintock J. B., Baker B. J. Spongian diterpenoids derived from the Antarctic sponge Dendrilla antarctica are potent inhibitors of the Leishmania parasite. J. Nat. Prod. 2020; 83:1553-1562. doi: 10.1021/acs.jnatprod.0c00025.
28. Kokkaliari S., Pham K., Shahbazi N., Calcul L., Wojtas L., Wilson N. G., Crawford A. D., Baker B. J. Australindolones, new aminopyrimidine substituted indolone alkaloids from an Antarctic tunicate Synoicum sp. Mar. Drugs. 2022; 20:196. doi: 10.3390/md20030196.
29. Illudalane Sesquiterpenoids of the Alcyopterosin Series from the Antarctic Marine Soft Coral Alcyonium grandis Marianna Carbone, Laura Núñez-Pons, Francesco Castelluccio, Conxita Avila, and Margherita Gavagnin Journal of Natural Products 2009 72 (7), 1357-1360, DOI: 10.1021/np900162t.

We claim:

1. A method of treating an infection of a protozoan parasite in a subject in need thereof, the method comprising: administering to the subject an effective amount of a composition comprising a compound, wherein the compound is one or more compounds selected from the group consisting of: 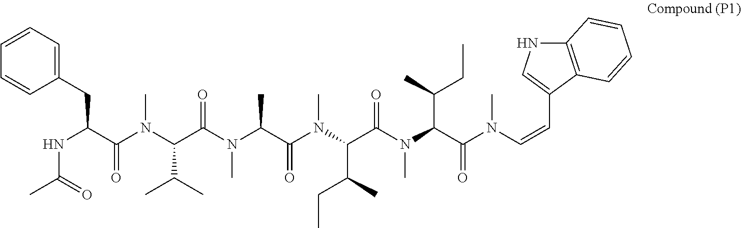 [SMILES: CC[C@H](C)[C@@H](C(=O)N(C)/C=C\c1c[nH]c2ccccc12)N(C)C(=O)[C@H]([C@@H](C)CC)N(C)C(=O)[C@H](C)N(C)C(=O)[C@H](C(C)C)N(C)C(=O)[C@H](Cc1ccccc1)NC(C)=O]
or a pharmaceutically acceptable salt thereof, wherein the configuration of the phenylalanyl group is L; 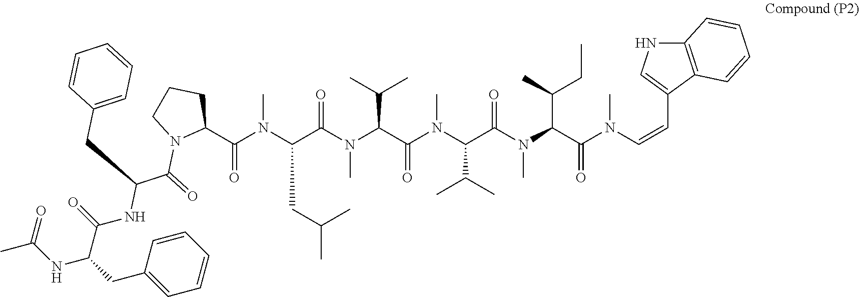 [SMILES: CC[C@H](C)[C@@H](C(=O)N(C)/C=C\c1c[nH]c2ccccc12)N(C)C(=O)[C@H](C(C)C)N(C)C(=O)[C@H](C(C)C)N(C)C(=O)[C@H](CC(C)C)N(C)C(=O)[C@@H]1CCCN1C(=O)[C@H](Cc1ccccc1)NC(=O)[C@H](Cc1ccccc1)NC(C)=O]
or a pharmaceutically acceptable salt thereof; 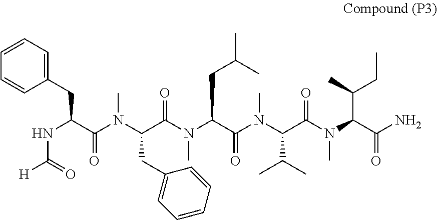 [SMILES: CC[C@H](C)[C@@H](C(N)=O)N(C)C(=O)[C@H](C(C)C)N(C)C(=O)[C@H](CC(C)C)N(C)C(=O)[C@H](Cc1ccccc1)N(C)C(=O)[C@H](Cc1ccccc1)NC=O]
or a pharmaceutically acceptable salt thereof; 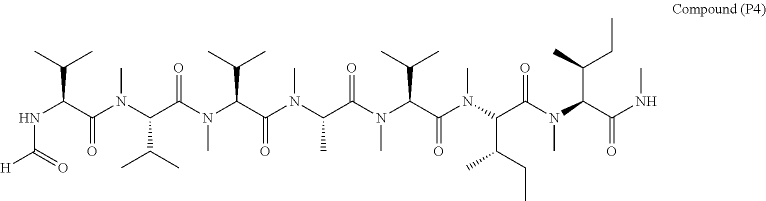
or a pharmaceutically acceptable salt thereof; 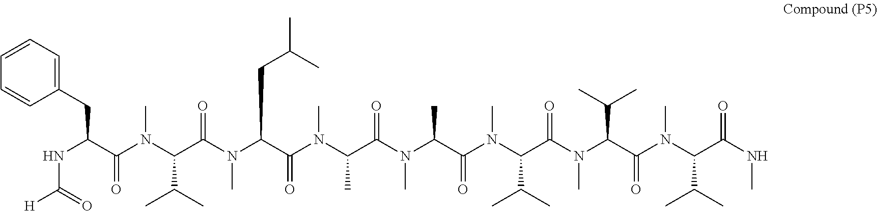 [SMILES: CNC(=O)[C@H](C(C)C)N(C)C(=O)[C@H](C(C)C)N(C)C(=O)[C@H](C(C)C)N(C)C(=O)[C@H](C)N(C)C(=O)[C@H](C)N(C)C(=O)[C@H](CC(C)C)N(C)C(=O)[C@H](C(C)C)N(C)C(=O)[C@H](Cc1ccccc1)NC=O]
or a pharmaceutically acceptable salt thereof; 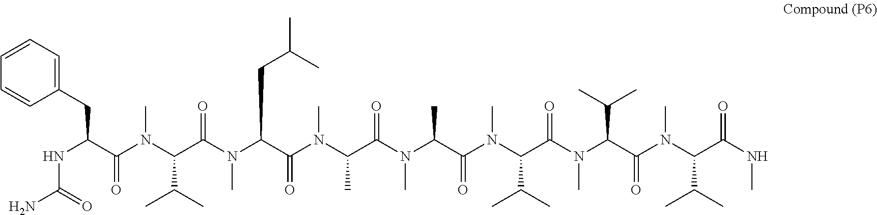 [SMILES: CNC(=O)[C@H](C(C)C)N(C)C(=O)[C@H](C(C)C)N(C)C(=O)[C@H](C(C)C)N(C)C(=O)[C@H](C)N(C)C(=O)[C@H](C)N(C)C(=O)[C@H](CC(C)C)N(C)C(=O)[C@H](C(C)C)N(C)C(=O)[C@H](Cc1ccccc1)NC(N)=O]
or a pharmaceutically acceptable salt thereof; 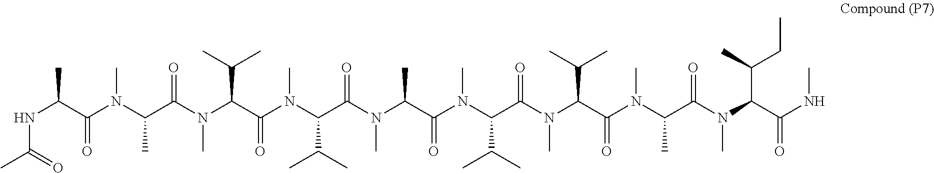 [SMILES: CC[C@H](C)[C@@H](C(=O)NC)N(C)C(=O)[C@H](C)N(C)C(=O)[C@H](C(C)C)N(C)C(=O)[C@H](C(C)C)N(C)C(=O)[C@H](C)N(C)C(=O)[C@H](C(C)C)N(C)C(=O)[C@H](C(C)C)N(C)C(=O)[C@H](C)N(C)C(=O)[C@H](C)NC(C)=O]
or a pharmaceutically acceptable salt thereof; and 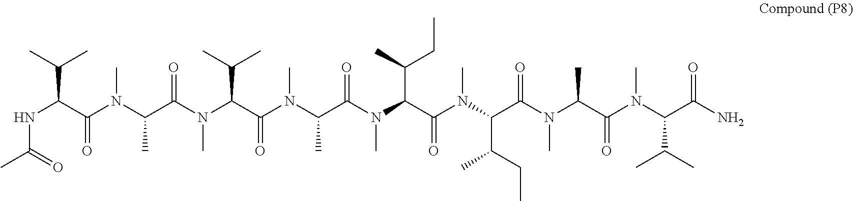 [SMILES: CC[C@H](C)[C@@H](C(=O)N(C)[C@@H](C)C(=O)N(C)[C@H](C(N)=O)C(C)C)N(C)C(=O)[C@H]([C@@H](C)CC)N(C)C(=O)[C@H](C)N(C)C(=O)[C@H](C(C)C)N(C)C(=O)[C@H](C)N(C)C(=O)[C@@H](NC(C)=O)C(C)C]
or a pharmaceutically acceptable salt thereof.

2.-9. (canceled)

10. The method of claim 1, wherein the protozoan parasite is Plasmodium falciparum.

11. The method of claim 10, wherein the protozoan parasite is a drug resistant strain of Plasmodium falciparum.

12. The method of claim 11, wherein the drug resistant strain of Plasmodium falciparum is resistant to artemisinin.

13.-15. (canceled)

16. A method of treating infection of a bacterium or a protist in a subject in need thereof, the method comprising: administering to the subject an effective amount of a composition comprising a compound, wherein the compound comprises one or more compounds selected from a group consisting of:
(a) compounds having a structure of formula I 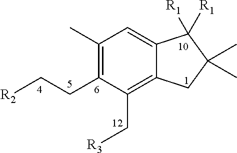
or a pharmaceutically acceptable salt thereof,
wherein R₁═H or a C-10 alkyl comprising a ketone,
wherein R₂═ONO₂ or OAc, and
wherein R₃═H, OAc, or OH;
(b) compounds having a structure of formula II 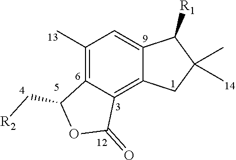
or a pharmaceutically acceptable salt thereof,
wherein R₁═H or OH, and
wherein R₂═OH, ONO₂, or Cl; or
(c) alcyosterone 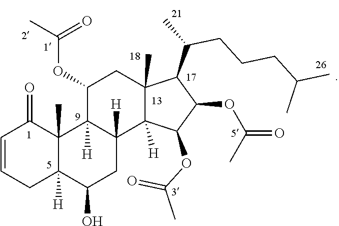
or a pharmaceutically acceptable salt thereof, or a combination thereof.

17. The method of claim 16, wherein the compound in the composition consists of the compound having the structure of formula I or the pharmaceutically acceptable salt thereof.

18. The method of claim 16, wherein the compound in the composition consists of the compound having the structure of formula II or a pharmaceutically acceptable salt thereof.

19. The method of claim 16, wherein the compound in the composition consists of alcyosterone or a pharmaceutically acceptable salt thereof.

20. The method of claim 17, wherein R₁═H, R₂═ONO₂, and R₃═OAC.

21. The method of claim 17, wherein R₁═C-10 alkyl comprising a ketone, R₂═ONO₂ and R₃═OAC.

22. The method of claim 17, wherein R₁═C-10 alkyl comprising a ketone, R₂=ONO₂ and R₃═H.

23. The method of claim 17, wherein R₁═H, R₂═ONO₂ and R₃═OH.

24. The method of claim 17, wherein R₁═H, R₂═OAc and R₃═OAc.

25. The method of claim 18, wherein R₁═H, and R₂═OH.

26. The method of claim 18, wherein R₁═H, and R₂═ONO₂.

27. The method of claim 18, wherein R₁═OH, and R₂═Cl.

28. The method of claim 16, wherein the infection comprises a bacterial infection.

29. The method of claim 28, wherein the bacteria is Clostridium difficile.

30. The method of claim 16, wherein the infection comprises a protist.

31. The method of claim 30, wherein the protist is Leishmania donovani.

32.-35. (canceled)
